(19) United States
(12) Patent Application Publication
(10) Pub. No.: US 2026/0265410 A1
(43) Pub. Date: Sep. 10, 2026

(54) RABBIT MONOCLONAL ANTIBODIES TARGETING ACUTE MYELOID LEUKEMIA ANTIGENS

(71) Applicant: UNIVERSITY OF FLORIDA RESEARCH FOUNDATION, INCORPORATED, Gainesville, FL (US)

(72) Inventors: Koji HASHIMOTO; Haiyong PENG, Jupiter, FL (US); Christoph RADER, Jupiter, FL (US)

(21) Appl. No.: 19/167,306

(22) PCT Filed: Mar. 18, 2024
(86) PCT No.: PCT/US2024/020419

§ 371 Date: Sep. 19, 2025

Related U.S. Application Data
(60) Provisional application No. 63/491,698, filed on Mar. 22, 2023.

(51) Int. Cl. C07K 16/40; A61K 40/31; A61K 47/68

(52) U.S. Cl. CPC C07K 16/40; A61K 40/31; A61K 47/6817; A61K 47/6871; A61K 2239/13; A61K 2239/21; A61K 2239/22; C07K 2317/24; C07K 2317/31; C07K 2317/52; C07K 2317/565

(57) ABSTRACT

The invention provides antibodies, antibody fragments or antigen-binding fragments, as well as related antibody drug conjugates (ADCs) and chimeric antigen receptors (CARs), that specifically recognize an acute myeloid leukemia (AML) cell surface antigen selected from PTPRG, Nectin-1, and endoglin. Also provided in the invention are methods of using such antibodies in various diagnostic and therapeutic applications for hematologic malignancies including acute myeloid leukemia (AML).

### CROSS REFERENCE TO RELATED APPLICATION

This application claims the benefit under 35 USC 119(e) of U.S. Provisional Application No. 63/491,698, filed Mar. 22, 2023, which is incorporated by reference in its entirety for all purposes.

### FEDERALLY SPONSORED RESEARCH OR DEVELOPMENT

This invention was made with government support under Grant No.'s R01 CA174844, R01 CA181258, R01 CA204484, and R21 CA263240 awarded by the National Institutes of Health. The government has certain rights in the invention.

### REFERENCE TO A SEQUENCE LISTING

The Sequence Listing written in file 607580SEQLST.xml is 133.9 kilobytes, was created on Jan. 25, 2024 and is hereby incorporated by reference.

### BACKGROUND OF THE INVENTION

Acute myeloid leukemia (AML) is a hematologic malignancy characterized by excessive growth of immature white blood cells (myeloblasts) in the bone marrow and in circulation, intrinsically affecting the process of hematopoiesis. The disease progresses rapidly and fatigue, shortness of breath, easy bruising, and bleeding, along with a greater risk of infection and dissemination of myeloblasts to the gums, skin, and brain, may occur as the disease progresses. Current treatments for AML include radiation therapy, stem cell transplant, anthracycline and cytarabine-based chemotherapy, IDH inhibitors (olutasidenib, ivosidenib, enasidenib), FLT3 inhibitors (gilteritinib, midostaurin), BCL-2 inhibitor (venetoclax), hypomethylating agents (azacitidine, decitabine), and CPX-351 (liposomal cytarabine and daunorubicin), however intensive chemotherapy may not be tolerated by older or frail patients. Immunotherapies available include gemtuzumab ozogamicin (GO), a CD33 monoclonal antibody conjugated to calicheamicin, as well as combination therapies [Totiger, T. M. et al., Biomedicines. 2023 February; 11(2): 641; Shimony, S. et al., American Journal of Hematology Volume 98, Issue 3 p. 502-526. Morse, J. S. et al. Expert Opin Investig Drugs. 2023 February; 32(2):107-125.].

Monoclonal antibody (mAb)-based drugs have revolutionized the therapy of cancer, autoimmune, inflammatory, infectious, and other diseases. As of April 2022, there have been 111 approvals of mAb-based therapeutics by the Food and Drug Administration (FDA) [1]. Approximately 50 mAb-based cancer therapies (including chimeric antigen receptor (CAR)-T therapies) have been FDA-approved as of March 2023. Despite this success, approved mAbs only target less than 25 unique receptors targeted on cancer cells—a small fraction of the roughly 5,000 cell surface proteins identified in humans, a number which neglects to capture the additional diversity gained by alternative splicing, post-translational modifications (PTMs). Furthermore, many approved mAbs have side effects due to on-target-off-tissue toxicity, highlighting the need for targets with more restricted expression to afford higher therapeutic indices.

One powerful tool that has enabled discovery of clinically and commercially successful antibody drugs is phage display [2-4]. The simple biology of phagemid/phage systems allows for monovalent display of antibody fragments (e.g. Fabs) via fusion to the N-terminus of the minor capsid protein pIII (or a C-terminal fragment thereof) and the fishing out of antibodies with unique reactivity that can then be determined by sequencing the phagemid DNA. Phage display selection of antibody libraries has several advantages over the traditional animal immunization including lower cost, faster results, and circumvention of immune tolerance[5]. Since many membrane proteins prove difficult to express recombinantly and purify, strategies have been developed to select antibody-phage libraries on the surface of whole cells where the target is in situ in its native conformation [6, 7]. Such selection strategies have required creative solutions to avoid selecting antibodies that bind to all cells or common cell surface proteins including depletion with cells or membrane vesicles that lack the target of interest [8, 9].

Given the unparalleled diversity in Fab-phage libraries and their facile in vitro selection, they offer the opportunity for target-agnostic selection on whole cells (whole-cell panning, WCP) to simultaneously identify both the antibody and the prospective target [6, 9, 10]. This antibody-driven drug discovery approach is unbiased and not limited to our current understanding of cell surface molecules as simply annotated membrane proteins, but may identify mAbs targeting cancer cell-specific isoforms, conformations[11], multiprotein complexes, glycosylation [12], or surface RNAs[13], to allow for biological discoveries [14]. However, technical aspects of phage biology such as the carryover and competitive growth of “bald” phage that lack antibody expression have limited the functional library selection depth [15, 16]. Another problem common to all target-agnostic drug discovery efforts is the identification of the target. In the case of antibodies, immunoprecipitation (IP) is the traditional method of choice, yet it does not work universally and can require extensive optimization, making it non-ideal for the parallel deorphanization of multiple candidate mAbs. Protein arrays, in which individual membrane proteins are overexpressed on the cell surface, provide useful information for on- and off-target interactions but are expensive and limited to one or two cell types, which express endogenous surface proteins resulting in high background and the inability to identify specific antigens [17, 18].

Therefore, there is a need for antibodies to AML cell surface antigens that can be used as a basis for the development of antibody-based targeted therapies of hematologic malignancies including AML. There is also a need for additional diagnostic tools for detecting AML cell surface antigen expression in hematologic malignancies including AML, e.g. such as Western-blotting and or immunohistochemistry (IHC). The instant invention is directed to addressing these and other needs.

### SUMMARY OF THE INVENTION

In one aspect, the invention provides an isolated antibody that competes for binding to an acute myeloid leukemia (AML) cell surface antigen with antibody P11, P17, P28, P130, P240, P1187, P1707, P1955, P2388, P2653, P2861, P3208, N277, N350, N484, N2282, N2430, or E2810. Some antibodies bind to the same epitope on an AML cell surface antigen as antibody P11, P17, P28, P130, P240, P1187, P1707, P1955, P2388, P2653, P2861, P3208, N277, N350, N484, N2282, N2430, or E2810. Some antibodies bind an AML cell surface antigen that is PTPRG, Nectin-1, or endoglin.

In another aspect, the antibody specifically binds PTPRG and comprises three heavy chain CDRs and three light chain CDRs of antibody P11, wherein P11 is characterized by a mature heavy chain variable region having an amino acid sequence comprising SEQ ID NO: 1 and a mature light chain variable region having an amino acid sequence comprising SEQ ID NO:2; comprises three heavy chain CDRs and three light chain CDRs of antibody P17, wherein P17 is characterized by a mature heavy chain variable region having an amino acid sequence comprising SEQ ID NO:9 and a mature light chain variable region having an amino acid sequence comprising SEQ ID NO: 10; comprises three heavy chain CDRs and three light chain CDRs of antibody P28, wherein P28 is characterized by a mature heavy chain variable region having an amino acid sequence comprising SEQ ID NO:17 and a mature light chain variable region having an amino acid sequence comprising SEQ ID NO:18; comprises three heavy chain CDRs and three light chain CDRs of antibody P130, wherein P130 is characterized by a mature heavy chain variable region having an amino acid sequence comprising SEQ ID NO:25 and a mature light chain variable region having an amino acid sequence comprising SEQ ID NO:26; comprises three heavy chain CDRs and three light chain CDRs of antibody P240, wherein P240 is characterized by a mature heavy chain variable region having an amino acid sequence comprising SEQ ID NO:33 and a mature light chain variable region having an amino acid sequence comprising SEQ ID NO:34; comprises three heavy chain CDRs and three light chain CDRs of antibody P1187, wherein P1187 is characterized by a mature heavy chain variable region having an amino acid sequence comprising SEQ ID NO:41 and a mature light chain variable region having an amino acid sequence comprising SEQ ID NO:42; comprises three heavy chain CDRs and three light chain CDRs of antibody P1707, wherein P1707 is characterized by a mature heavy chain variable region having an amino acid sequence comprising SEQ ID NO:49 and a mature light chain variable region having an amino acid sequence comprising SEQ ID NO:50; comprises three heavy chain CDRs and three light chain CDRs of antibody P1955, wherein P1955 is characterized by a mature heavy chain variable region having an amino acid sequence comprising SEQ ID NO:57 and a mature light chain variable region having an amino acid sequence comprising SEQ ID NO:58; comprises three heavy chain CDRs and three light chain CDRs of antibody P2388, wherein P2388 is characterized by a mature heavy chain variable region having an amino acid sequence comprising SEQ ID NO:65 and a mature light chain variable region having an amino acid sequence comprising SEQ ID NO:66; comprises three heavy chain CDRs and three light chain CDRs of antibody P2653, wherein P2653 is characterized by a mature heavy chain variable region having an amino acid sequence comprising SEQ ID NO:73 and a mature light chain variable region having an amino acid sequence comprising SEQ ID NO:74; comprises three heavy chain CDRs and three light chain CDRs of antibody P2861, wherein P2861 is characterized by a mature heavy chain variable region having an amino acid sequence comprising SEQ ID NO:81 and a mature light chain variable region having an amino acid sequence comprising SEQ ID NO:82; or comprises three heavy chain CDRs and three light chain CDRs of antibody P3208, wherein P3208 is characterized by a mature heavy chain variable region having an amino acid sequence comprising SEQ ID NO:89 and a mature light chain variable region having an amino acid sequence comprising SEQ ID NO:90.

In some antibodies, the CDRs are of a definition selected from the group consisting of Kabat, Chothia, Kabat/Chothia Composite, AbM, Contact, and IMGT.

In some antibodies, the mature heavy chain variable region comprises the three Kabat heavy chain CDRs of SEQ ID NOs:3-5 and the mature light chain variable region comprises the three Kabat light chain CDRs of SEQ ID NOs:6-8; the mature heavy chain variable region comprises the three Kabat heavy chain CDRs of SEQ ID NOs:11-13 and the mature light chain variable region comprises the three Kabat light chain CDRs of SEQ ID NOs:14-16; the mature heavy chain variable region comprises the three Kabat heavy chain CDRs of SEQ ID NOs:19-21 and the mature light chain variable region comprises the three Kabat light chain CDRs of SEQ ID NOs:22-24; the mature heavy chain variable region comprises the three Kabat heavy chain CDRs of SEQ ID NOs:27-29 and the mature light chain variable region comprises the three Kabat light chain CDRs of SEQ ID NOs:30-32; the mature heavy chain variable region comprises the three Kabat heavy chain CDRs of SEQ ID NOs:35-37 and the mature light chain variable region comprises the three Kabat light chain CDRs of SEQ ID NOs:38-40; the mature heavy chain variable region comprises the three Kabat heavy chain CDRs of SEQ ID NOs:43-45 and the mature light chain variable region comprises the three Kabat light chain CDRs of SEQ ID NOs:46-48; the mature heavy chain variable region comprises the three Kabat heavy chain CDRs of SEQ ID NOs:51-53 and the mature light chain variable region comprises the three Kabat light chain CDRs of SEQ ID NOs:54-56; the mature heavy chain variable region comprises the three Kabat heavy chain CDRs of SEQ ID NOs:59-61 and the mature light chain variable region comprises the three Kabat light chain CDRs of SEQ ID NOs:62-64; the mature heavy chain variable region comprises the three Kabat heavy chain CDRs of SEQ ID NOs:67-69 and the mature light chain variable region comprises the three Kabat light chain CDRs of SEQ ID NOs:70-72; the mature heavy chain variable region comprises the three Kabat heavy chain CDRs of SEQ ID NOs:75-77 and the mature light chain variable region comprises the three Kabat light chain CDRs of SEQ ID NOs:78-80; the mature heavy chain variable region comprises the three Kabat heavy chain CDRs of SEQ ID NOs:83-85 and the mature light chain variable region comprises the three Kabat light chain CDRs of SEQ ID NOs:86-88; or the mature heavy chain variable region comprises the three Kabat heavy chain CDRs of SEQ ID NOs:91-93 and the mature light chain variable region comprises the three Kabat light chain CDRs of SEQ ID NOs:94-96;

Some antibodies comprise a mature heavy chain variable region having an amino acid sequence at least 90% identical to SEQ ID NO:1, and a mature light chain variable region having an amino acid sequence at least 90% identical to SEQ ID NO:2; comprise a mature heavy chain variable region having an amino acid sequence at least 90% identical to SEQ ID NO:9, and a mature light chain variable region having an amino acid sequence at least 90% identical to SEQ ID NO:10; comprise a mature heavy chain variable region having an amino acid sequence at least 90% identical to SEQ ID NO:17, and a mature light chain variable region having an amino acid sequence at least 90% identical to SEQ ID NO:18; comprise a mature heavy chain variable region having an amino acid sequence at least 90% identical to SEQ ID NO:25, and a mature light chain variable region having an amino acid sequence at least 90% identical to SEQ ID NO:26; comprise a mature heavy chain variable region having an amino acid sequence at least 90% identical to SEQ ID NO:33, and a mature light chain variable region having an amino acid sequence at least 90% identical to SEQ ID NO:34; comprise a mature heavy chain variable region having an amino acid sequence at least 90% identical to SEQ ID NO:41, and a mature light chain variable region having an amino acid sequence at least 90% identical to SEQ ID NO:42; comprise a mature heavy chain variable region having an amino acid sequence at least 90% identical to SEQ ID NO:49, and a mature light chain variable region having an amino acid sequence at least 90% identical to SEQ ID NO:50; comprise a mature heavy chain variable region having an amino acid sequence at least 90% identical to SEQ ID NO:57, and a mature light chain variable region having an amino acid sequence at least 90% identical to SEQ ID NO:58; comprise a mature heavy chain variable region having an amino acid sequence at least 90% identical to SEQ ID NO:65, and a mature light chain variable region having an amino acid sequence at least 90% identical to SEQ ID NO:66; comprise a mature heavy chain variable region having an amino acid sequence at least 90% identical to SEQ ID NO:73, and a mature light chain variable region having an amino acid sequence at least 90% identical to SEQ ID NO:74; comprise a mature heavy chain variable region having an amino acid sequence at least 90% identical to SEQ ID NO:81, and a mature light chain variable region having an amino acid sequence at least 90% identical to SEQ ID NO:82; or comprise a mature heavy chain variable region having an amino acid sequence at least 90% identical to SEQ ID NO:89, and a mature light chain variable region having an amino acid sequence at least 90% identical to SEQ ID NO:90.

In some antibodies, the mature heavy chain variable region has an amino acid sequence of SEQ ID NO:1 and the mature light chain variable region has an amino acid sequence of SEQ ID NO:2; the mature heavy chain variable region has an amino acid sequence of SEQ ID NO:9 and the mature light chain variable region has an amino acid sequence of SEQ ID NO:10; the mature heavy chain variable region has an amino acid sequence of SEQ ID NO:17 and the mature light chain variable region has an amino acid sequence of SEQ ID NO:18; the mature heavy chain variable region has an amino acid sequence of SEQ ID NO:25 and the mature light chain variable region has an amino acid sequence of SEQ ID NO:26; the mature heavy chain variable region has an amino acid sequence of SEQ ID NO:33 and the mature light chain variable region has an amino acid sequence of SEQ ID NO:34; the mature heavy chain variable region has an amino acid sequence of SEQ ID NO:41 and the mature light chain variable region has an amino acid sequence of SEQ ID NO:42; the mature heavy chain variable region has an amino acid sequence of SEQ ID NO:49 and the mature light chain variable region has an amino acid sequence of SEQ ID NO:50; the mature heavy chain variable region has an amino acid sequence of SEQ ID NO:57 and the mature light chain variable region has an amino acid sequence of SEQ ID NO:58; the mature heavy chain variable region has an amino acid sequence of SEQ ID NO:65 and the mature light chain variable region has an amino acid sequence of SEQ ID NO:66; the mature heavy chain variable region has an amino acid sequence of SEQ ID NO:73 and the mature light chain variable region has an amino acid sequence of SEQ ID NO:74; the mature heavy chain variable region has an amino acid sequence of SEQ ID NO:81 and the mature light chain variable region has an amino acid sequence of SEQ ID NO:82; or the mature heavy chain variable region has an amino acid sequence of SEQ ID NO:89 and the mature light chain variable region has an amino acid sequence of SEQ ID NO:90.

In another aspect, the antibody specifically binds Nectin-2 and comprises three heavy chain CDRs and three light chain CDRs of antibody N277, wherein N277 is characterized by a mature heavy chain variable region having an amino acid sequence comprising SEQ ID NO:97 and a mature light chain variable region having an amino acid sequence comprising SEQ ID NO:98; comprises three heavy chain CDRs and three light chain CDRs of antibody N350, wherein N350 is characterized by a mature heavy chain variable region having an amino acid sequence comprising SEQ ID NO: 105 and a mature light chain variable region having an amino acid sequence comprising SEQ ID NO:106; comprises three heavy chain CDRs and three light chain CDRs of antibody N484, wherein N484 is characterized by a mature heavy chain variable region having an amino acid sequence comprising SEQ ID NO:113 and a mature light chain variable region having an amino acid sequence comprising SEQ ID NO: 114; comprises three heavy chain CDRs and three light chain CDRs of antibody N2282, wherein N2282 is characterized by a mature heavy chain variable region having an amino acid sequence comprising SEQ ID NO:121 and a mature light chain variable region having an amino acid sequence comprising SEQ ID NO: 122; or comprises three heavy chain CDRs and three light chain CDRs of antibody N2430, wherein N2430 is characterized by a mature heavy chain variable region having an amino acid sequence comprising SEQ ID NO:129 and a mature light chain variable region having an amino acid sequence comprising SEQ ID NO:130.

In some such antibodies, the CDRs are of a definition selected from the group consisting of Kabat, Chothia, Kabat/Chothia Composite, AbM, Contact, and IMGT.

In some antibodies the mature heavy chain variable region comprises the three Kabat heavy chain CDRs of SEQ ID NOs:99-101 and the mature light chain variable region comprises the three Kabat light chain CDRs of SEQ ID NOs:102-104; the mature heavy chain variable region comprises the three Kabat heavy chain CDRs of SEQ ID NOs:107-109 and the mature light chain variable region comprises the three Kabat light chain CDRs of SEQ ID NOs:110-112; the mature heavy chain variable region comprises the three Kabat heavy chain CDRs of SEQ ID NOs:115-117 and the mature light chain variable region comprises the three Kabat light chain CDRs of SEQ ID NOs:118-120; the mature heavy chain variable region comprises the three Kabat heavy chain CDRs of SEQ ID NOs:123-125 and the mature light chain variable region comprises the three Kabat light chain CDRs of SEQ ID NOs:126-128; or the mature heavy chain variable region comprises the three Kabat heavy chain CDRs of SEQ ID NOs:131-133 and the mature light chain variable region comprises the three Kabat light chain CDRs of SEQ ID NOs:134-136.

Some antibodies comprise a mature heavy chain variable region having an amino acid sequence at least 90% identical to SEQ ID NO:97, and a mature light chain variable region having an amino acid sequence at least 90% identical to SEQ ID NO:98; comprise a mature heavy chain variable region having an amino acid sequence at least 90% identical to SEQ ID NO: 105, and a mature light chain variable region having an amino acid sequence at least 90% identical to SEQ ID NO:106; comprise a mature heavy chain variable region having an amino acid sequence at least 90% identical to SEQ ID NO:113, and a mature light chain variable region having an amino acid sequence at least 90% identical to SEQ ID NO:114; comprise a mature heavy chain variable region having an amino acid sequence at least 90% identical to SEQ ID NO:121, and a mature light chain variable region having an amino acid sequence at least 90% identical to SEQ ID NO:122; or comprise a mature heavy chain variable region having an amino acid sequence at least 90% identical to SEQ ID NO:129, and a mature light chain variable region having an amino acid sequence at least 90% identical to SEQ ID NO:130.

In some antibodies, the mature heavy chain variable region has an amino acid sequence of SEQ ID NO:97 and the mature light chain variable region has an amino acid sequence of SEQ ID NO:98; the mature heavy chain variable region has an amino acid sequence of SEQ ID NO:105 and the mature light chain variable region has an amino acid sequence of SEQ ID NO:106; the mature heavy chain variable region has an amino acid sequence of SEQ ID NO:113 and the mature light chain variable region has an amino acid sequence of SEQ ID NO:114; the mature heavy chain variable region has an amino acid sequence of SEQ ID NO:121 and the mature light chain variable region has an amino acid sequence of SEQ ID NO:122; or the mature heavy chain variable region has an amino acid sequence of SEQ ID NO:129 and the mature light chain variable region has an amino acid sequence of SEQ ID NO:130.

In another aspect, the antibody specifically binds endoglin and comprises three heavy chain CDRs and three light chain CDRs of antibody E2810, wherein E2810 is characterized by a mature heavy chain variable region having an amino acid sequence comprising SEQ ID NO:137 and a mature light chain variable region having an amino acid sequence comprising SEQ ID NO:138.

In some such antibodies, the CDRs are of a definition selected from the group consisting of Kabat, Chothia, Kabat/Chothia Composite, AbM, Contact, and IMGT.

In some such antibodies, the mature heavy chain variable region comprises the three Kabat heavy chain CDRs of SEQ ID NOs:139-141 and the mature light chain variable region comprises the three Kabat light chain CDRs of SEQ ID NOs:142-144.

Some antibodies comprise a mature heavy chain variable region having an amino acid sequence at least 90% identical to SEQ ID NO:137, and a mature light chain variable region having an amino acid sequence at least 90% identical to SEQ ID NO:138.

In some antibodies, the mature heavy chain variable region has an amino acid sequence of SEQ ID NO:137 and the mature light chain variable region has an amino acid sequence of SEQ ID NO:138.

Some antibodies are chimeric or humanized.

Some antibodies are IgAQ1, IgA2, IgD, IgE, IgG1, IgG2, IgG3, IgG4, synthetic IgG, IgM, F(ab)2, Fv, scFv, IgGACH2, F(ab′)2, scFv2CH3, Fab, VL, VH, scFv4, scFv3, scFv2, dsFv, Fv, scFv-Fc, (scFv)2, a non-depleting IgG, a diabody, or a bivalent antibody.

Some antibodies have the mature light chain variable region fused to a light chain constant region and the mature heavy chain variable region fused to a heavy chain constant region. The heavy chain constant region of some antibodies is a mutant form of a natural human heavy chain constant region which has reduced binding to a Fcγ receptor relative to the natural human heavy chain constant region.

The antibody can be an antibody fragment. The antibody can be a bispecific antibody. The bispecific antibody can be a T-cell engaging bispecific antibody or an NK-cell engaging bispecific antibody. The antibody can be an antibody-based binding protein.

Some antibodies are conjugated to a synthetic molecule. In some embodiments, the synthetic molecule is a cytotoxic agent, a label, a therapeutic radioisotope, or a liposome. In some embodiments, the cytotoxic agent is a small molecule weight toxin, a peptide toxin, or a protein toxin.

In another aspect, any of the antibodies disclosed herein is linked to at least one cytotoxic agent as an antibody drug conjugate. In some embodiments, the cytotoxic agent is a small molecular weight toxin, a peptide toxin, or a protein toxin. In some embodiments, the antibody is chimeric or humanized.

34. In another aspect, the invention provides a chimeric antigen receptor (CAR), comprising any of the antibodies disclosed herein fused to a transmembrane region and an intracellular T-cell receptor (TCR) signaling domain, an intracellular T-cell costimulatory receptor signaling domain, or a signaling domain of a protein downstream of TCR or T-cell costimulatory receptor. In some chimeric antigen receptors, the antibody is chimeric or humanized.

In another aspect, the invention provides a pharmaceutical composition comprising a therapeutically effective amount of any of the antibodies disclosed herein and a pharmaceutically acceptable carrier.

In another aspect, the invention provides a kit comprising any of the antibodies disclosed herein.

In another aspect, the invention provides a nucleic acid encoding the heavy chain and/or light chain of any of the antibodies disclosed herein and a recombinant expression vector comprising the nucleic acid.

In yet another aspect, the invention provides a method of killing or inhibiting the growth of a cell expressing an acute myeloid leukemia cell surface antigen in a subject, comprising administering a pharmaceutical composition disclosed herein to a subject in need thereof, thereby killing or inhibiting the growth of the cell expressing the acute myeloid leukemia cell surface antigen in the subject. In some methods, the cell is a tumor cell.

In some methods, the pharmaceutical composition comprises an antibody drug conjugate (ADC) comprising any of the antibodies disclosed herein and a cytotoxic agent.

In some methods, the cytotoxic agent is a small molecular weight toxin, a peptide toxin, or a protein toxin.

In yet another aspect, the invention provides a method of treating a hematologic malignancy in a subject, comprising administering a pharmaceutical composition disclosed herein to the subject having the hematologic malignancy, thereby treating the hematologic malignancy in the subject. In some methods, the hematologic malignancy is acute myeloid leukemia.

In some methods, the pharmaceutical composition comprises an antibody drug conjugate (ADC) comprising any of the antibodies disclosed herein and a cytotoxic agent. In some methods, the cytotoxic agent is a small molecular weight toxin, a peptide toxin, or a protein toxin.

In some methods, the antibody is fused to a transmembrane region and an intracellular T-cell receptor (TCR) signaling domain, an intracellular T-cell costimulatory receptor signaling domain, or a signaling domain of a protein downstream of TCR or T-cell costimulatory receptor to forn a chimeric antigen receptor (CAR).

A further understanding of the nature and advantages of the present invention may be realized by reference to the remaining portions of the specification and claims.

### DESCRIPTION OF THE DRAWINGS

FIG. 1 is a schematic of the steps in antibody discovery for acute myeloid leukemia.

FIGS. 2A and 2B depict binding of antibodies to cells. 2A, is a table showing binding of antibodies to AML cell lines and PBMC. 2B, shows results of flow cytometry studies showing binding of antibodies to Kasumi-1 cells and to HSC/P. In FIGS. 2A and 2B: Clones 484, 277, 2282, 350, and 2430 (boldface group); Clones 11, 17, 28, 43, 64, 130, 240, 890, 1707, 1955, 2385, 2388, 2653, 2861, 3208, and 1187 (angled-lines shading group).

FIGS. 3A-E depict Target deconvolution of mAb_11 by screening of CRISPR library. 3A. Results of studies of HEL cells transduced with CRISPR gRNA library sorted by IgG_11 to enrich antigen-negative cells due to gRNA-guided knockout by Cas9. Left hand panel shows results before sorting, middle panel shows results after first round of sorting, and righthand panel shows results after second round of sorting. 3B. Results of studies where Antigen-negative cells after sorting were collected to extract genomic DNA for PCR to amplify gRNA integrated into genome, followed by NGS and MAGeCK analysis to identify top gRNAs that could guide Cas9 to knock out the target antigen. Plot shows PTPRG ranked top 1 for mAb_11. 3C. Plot showing that 4 out of 5 gRNAs targeting PTPRG had been significantly enriched. 3D. Flow cytometry showing binding signal of Fab_11 to U937 cells transduced with Top1 gRNA from FIG. 3C (PTPRG gRNA-1), another gRNA targeting PTPRG (PTPRG gRNA-zf) chosen randomly from one of the 8 gRNAs targeting PTPRG from a different CRISPR-gRNA library (GeCKO v2 library, Sanjana et al Nat Methods. 2014 August; 11(8):783-4) (PTPRG gRNA-zf), both Top1 gRNA and another gRNA co-transduced ((PTPRG gRNA-dKO, Top1 gRNA from 3C and PTPRG gRNA-zf)), control APCS gRNA-2, compared with parental U937 cells (U937), U937 cells treated with secondary antibody only (2^{nd} only), and U937 cells unstained, 3E. SPR data showing the affinity of Fab_11 to human PTPRG ECD fused to hFc (hPTPRG-ECD-hFc) (K_{D}=4.23×10⁻⁹ M)

FIGS. 4A-B depict Target evaluation of mAb_11 by flow cytometry and SPR. 4A. Flow cytometry showing binding signal of IgG_11 in U937 cells treated with recombinant PTPRG protein (human PTPRG N-terminal domain) (left panel) or treated with rabbit polyclonal antibodies targeting human PTPRG (rbAb) (right panel) in a concentration-dependent manner. Plots from top to bottom of FIG. 4A: (U937 cells treated with 0.1 μg of IgG_11 and 1 μg of hPTPRG-NTD or rbAb, as indicated), (U937 cells treated with 0.1 μg of IgG_11 and 0.1 μg of hPTPRG-NTD or rbAb, as indicated), (U937 cells treated with 0.1 μg IgG_11 only), (U937 cells treated with secondary antibody (2^{nd} Ab) only), (U937 cells unstained) 4B. Plots of SPR data showing Fab_11 binds the extracellular domain of mouse PTPRG (mPTPRG-ECD;) (K_{D}=3.31×10⁻⁹ M) and the N-terminal domain of human PTPRG (hPTPRG-NTD) (K_{D}=4.73×10⁻⁹ M) with high affinities.

FIGS. 5A-B depict Target deconvolution of other clones in the angled-lines shading group (of FIGS. 2A and 2B) by ELISA and flow cytometry. 5A. Plot of ELISA binding of human PTPRG extracellular domain (hPTPRG-ECD, shaded with horizontal lines), human PTPRG N-terminal domain (hPTPRG-NTD, dotted shading), human PTPRG carboxyl domain (hPTPRG-CTD, solid shading), and mouse PTPRG extracellular domain (mPTPRG-ECD, shaded with vertical and horizontal lines) to clones in angled-lines shading group (of FIGS. 2A and 2B) group (Fab_1, Fab_17, Fab_28, Fab_64, Fab_130, Fab_240, Fab_1707, Fab_1955, Fab_2385, Fab_2388, Fab_2653, Fab_2861, and Fab_3208) compared with binding to Fab_484 (boldface group of FIGS. 2A and 2B). 5B. Epitope binning studies by flow cytometry measuring binding of biotinylated Fab_11 to cells expressing PTPRG on cell surface, when detected by streptavidin-PE, in competition with unbiotinylated Fab_3208, Fab_2861, Fab_2635, Fab_2388, Fab_1955, Fab_1707, Fab_240, Fab_130, Fab_64, Fab_28, Fab_17, and Fab_11). Also shown is binding of biotinylated Fab_11 to cells expressing PTPRG on cell surface, when detected by streptavidin-PE, with no unbiotinylated competitor Fab (bFab_11), and signal in U937 cells treated with secondary antibody only (2^{nd} only) and to unstained U937 cells.

FIGS. 6A-D depict Target deconvolution of mAb_1187, which also recognizes PTPRG. 6A. HEL parental cells (left panel) or HEL cells negatively sorted by IgG_11 (right panel) stained by biotinylated IgG_1187. 6B. Binding signals of biotinylated IgG_11 (bIgG_11, left panel) and biotinylated IgG_1187 (bIgG_1187, right panel) to HEL cells measured in presence of unbiotinylated Fab_1187, unbiotinylated Fab_11, unbiotinylated Fab_2810, unbiotinylated Fab_484, unbiotinylated Fab_2282) compared to binding signal with no unbiotinylated Fab (+0). Also shown is binding signal in HEL cells treated with streptavidin/R-Phycoerythrin only (SA/PE, and in untreated_HEL cells (HEL). 6C. Flow cytometry showing binding signal of IgG_1187 to U937 cells treated with recombinant PTPRG protein (human PTPRG N-terminal domain) (left panel) or treated with rabbit polyclonal antibodies targeting human PTPRG (rbAb) (right panel) in a concentration-dependent manner. Plots from top to bottom of FIG. 6C: (U937 cells treated with 0.1 μg of IgG_1187 and 1 μg of hPTPRG-NTD or rbAb, as indicated), (U937 cells treated with 0.1 μg of IgG_1187 and 0.1 μg of hPTPRG-NTD or rbAb, as indicated), (U937 cells treated with 0.1 μg IgG_11 only), (U937 cells treated with secondary antibody (Jackson Immunoresearch, cat #: 109-116-097) (2^{nd} Ab) only), (U937 cells unstained) 6D. Plot of SPR data showing the affinity of Fab_1187 to human PTPRG-ECD (K_{D}=1.90×10⁻⁷ M)

FIGS. 7A-E depict Target deconvolution of mAb_2282 in the boldface group (of FIGS. 2A and 2B) by screening of CRISPR library. 7A. Binding studies of U937 cells transduced with CRISPR gRNA library and sorted by Fab_2282 to enrich antigen-negative cells due to gRNA-guided knockout by Cas9. Flow cytometry showing binding signal, plots from top to bottom of FIG. 7A: after 2 rounds of sorting (marked as Fab_2282 Sorted 2^{nd}), after 1 round of sorting (marked as Fab_2282 Sorted 1^{st}), unsorted U937 cells (marked as U937 unsorted), and unstained U937 cells (marked as U937 unstained). 7B. Results of studies where antigen-negative cells after sorting were collected to extract genomic DNA for PCR to amplify gRNA integrated into genome, followed by NGS and MAGeCK analysis to identify top gRNAs that could guide Cas9 to knock out the target antigen. Plot shows PVRL2 (also named NECTIN2) ranked top 1 for mAb_2282. 7C. Plot showing that all 5 gRNAs targeting NECTIN2 had been significantly enriched. 7D. Flow cytometry showing binding signal of Fab_2282 to U937 cells transduced with Top1 gRNA from FIG. 7C (marked as g-RNA-1), another gRNA targeting NECTIN2 (marked as gRNA-zf) from a different CRISPR-gRNA library (GeCKO v2 library, Sanjana et al., supra), both Top1 gRNA from 7C and another gRNA from GeCKO v2 library co-transduced (marked as gRNA-dKO, top 1 gRNA from FIG. 7C and gRNA-zf) compared with parental U937 cells (U937), and U937 cells unstained. 7E. Plot of SPR data showing the affinity of Fab_2282 to human NECTIN2-ECD-hFc (K_{D}=5.97×10⁻⁷ M)

FIGS. 8A-D depict Target deconvolution of other clones in the boldface group (of FIGS. 2A and 2B) by competition flow cytometry. 8A. Studies measuring binding of 0.5 μg of Alexa 647 labeled Fab_2282 to U937 cells in competition with 5 μg unlabeled Fab_2810, Fab_2430, Fab_277, Fab_484, and Fab_2282. Also shown is binding of Alexa 647 labeled Fab_2282 U937 cells with no unlabeled competitor (marked as 0.5 μg Fab_2282-647), and signal in U937 cells treated with secondary antibody only (marked as 2^{nd} Ab) and to unstained U937 cells (marked as U937 unstained) 8B-8D. Flow cytometry showing binding signal of 0.5 μg of Fab_2282 (8B), Fab 484 (8C), and Fab_277 (8D) to U937 cells treated with recombinant Nectin-2-extracellular domain (ECD) fused to hFc (NECTIN2-ECD-hFc) (left panel of each of 8B-D) or treated with rabbit polyclonal antibodies targeting human Nectin-2 (rb pAb) (right panel of each of 8B-D). Plots from top to bottom of FIG. 8B-D: (U937 cells treated with 0.5 μg of indicated Fab and 5 μg of Nectin-2-ECD-hFc or with rb pAb, as indicated), (U937 cells treated with 0.5 μg of indicated Fab and 1 μg Nectin-2-ECD-hFc or with rb pAb, as indicated), (U937 cells treated with 0.5 μg indicated Fab only (U937 cells treated with secondary antibody (2^{nd} Ab) only), (U937 cells unstained)

FIGS. 9A-B depicts Target deconvolution of other clones in the boldface group (of FIGS. 2A and 2B) by CRISPR KO and SPR. 9A. For U937 cells transduced with Top 1 gRNA as in FIG. 7C (marked as gRNA-1) and another gRNA targeting NECTIN2 (marked as gRNA-zf) from a different CRISPR-gRNA library (GeCKO v2 library, Sanjana et al., supra) (, both Top1 gRNA from FIG. 7C and another gRNA targeting NECTIN2 from GeCKO v2 library co-transduced (marked as gRNA-dKO, top 1 gRNA as in FIG. 7C and gRNA-zf), parental U937 cells (marked as U937), and U937 cells unstained (marked as unstained), binding was measured to boldface group clones Fab_484, Fab_277, and Fab_2430, to Fab_2810 (control, not in boldface group, and to a commercial mAb targeting Nectin-2. 9B. Plot of SPR data showing the affinity of Fab_484, Fab_277, Fab_2430, and Fab_350 to human Nectin-2-ECD-hFc

FIGS. 10A-E depict Target deconvolution of mAb_2810 by screening of CRISPR library. 10A. Binding studies of U937 cells transduced with CRISPR gRNA library and sorted by Fab_2810 to enrich antigen-negative cells due to gRNA-guided knockout by Cas9. Plot shows binding signal after 2 rounds of sorting (marked as Fab_2810 Sorted 2^{nd}), after 1 round of sorting (marked as Fab_2810 Sorted 1^{st}), unsorted U937 cells (marked as U937 unsorted), and unstained U937 cells (marked as U937 unstained). 10B. Results of studies where antigen-negative cells after sorting were collected to extract genomic DNA for PCR to amplify gRNA integrated into genome, followed by NGS and MAGeCK analysis to identify top gRNAs that could guide Cas9 to knock out the target antigen. Plot shows that END (or CD105) ranked top 1 for mAb_2810. 10C. Plot showing all 5 gRNAs targeting CD105 had been significantly enriched. 10D. Flow cytometry showing binding signal of Fab_2810 to U937 cells transduced with Top1 gRNA from FIG. 10C (marked as g-RNA-1), another gRNA targeting CD105 (gRNA-zf) from a different CRISPR-gRNA library (GeCKO v2 library, Sanjana et al., supra) (marked as gRNA-zf), both Top1 gRNA from FIG. 10C and another gRNA targeting CD105 from GeCKO v2 library co-transduced (top1 gRNA from FIG. 10C and gRNA-zf, marked as gRNA-dKO) compared with parental U937 cells (marked as U937), and U937 cells unstained (marked as unstained). 10E. Plot of SPR data showing the affinity of Fab_2810 to human CD105-ECD (K_{D}=8.22×10⁻⁹ M)

FIGS. 11A-B depict Target evaluation of mAb_2810 by competition flow cytometry and CRISPR KO. 11A. Flow cytometry showing binding signal of Fab_2810 (labeled with Alexa 647) to U937 cells treated with recombinant human CD105-hFc domain (hCD105-hFc) (left panel) or treated with goat polyclonal antibody targeting human CD105 (plAb) (right panel). Plots from top to bottom of FIG. 11A: (U937 cells treated with 0.5 μg of Fab_2810 labeled with Alexa 647 and 5 μg of hCD105-hFc or of pIAb, as indicated), (U937 cells treated with 0.5 μg of Fab_2810 labeled with Alexa 647 and 1 ug hCD105-hFc or of pIAb, as indicated), (U937 cells treated with 0.5 ug Fab_2810 labeled with Alexa 647 only, Fab_2810-647), (U937 cells unstained). 11B. Flow cytometry showing binding signal of Commercial mAb targeting CD105 (antibody labeled with Alexa 647) to U937 cells cloned and transduced with Top1 gRNA as in FIG. 10C (marked as g-RNA-1), another gRNA targeting CD105 as in FIG. 10D (marked as gRNA-zf) from a different CRISPR-gRNA library (GeCKO v2 library, Sanjana et al., supra)), both Top1 gRNA from 10C and another gRNA targeting CD105 from GeCKO v2 library co-transduced (marked as gRNA-dKO, top1 gRNA from FIG. 10C and gRNA-zf)) compared with parental U937 cells (marked as U937), and U937 cells unstained (marked as unstained).

FIGS. 12A and 12B depict Specificity and affinity of new mAbs to the corresponding antigens. 12A. ELISA data of binding of antibodies Fab_11, Fab_130, Fab_64, Fab_1955, Fab_484, Fab_2282, Fab_277, Fab_2430, Fab_2810, and control antibody (Fab_Ctrl) to each of PTPRG (shaded with horizontal lines), Nectin-2 (solid fill), and CD105 (no fill). 12B. Table summarizing affinities for listed antibodies. (hPTRPG-ECD refers to human PTPRG extracellular domain; mPTPRG-ECD refers to mouse PTPRG extracellular domain; hPTPRG-NTD refers to human PTPRG N-terminal domain, hNectin-2 -ECD refers to human Nectin-2 extracellular domain, hCD015-ECD refers to human CD105 extracellular domain).

FIGS. 13A-13D depict an amino acid sequence alignment of the rabbit variable domains (V_{H} and V_{λ},) of chimeric rabbit/human Fab clones is shown with framework regions (FR) and complementarity determining regions (CDR) using Kabat numbering. CDRs shown are of Kabat definition. Clones starting with P bind PTPRG, clones starting with N bind Nectin-2, and clone starting with E binds CD105/endoglin. Sequence identifiers for sequences in FIGS. 13A-13B are presented in Table 2. 13A-13B (V_{H}); 13C-13D (V_{λ})

### BRIEF DESCRIPTION OF THE SEQUENCES

SEQ ID NO:1 sets forth the amino acid sequence of mature heavy chain variable region of clone P11
SEQ ID NO:2 sets forth the amino acid sequence of mature light chain variable region of clone P11
SEQ ID NO:3 sets forth the P11 Kabat CDRH1 amino acid sequence
SEQ ID NO:4 sets forth the P11 Kabat CDR12 amino acid sequence
SEQ ID NO:5 sets forth the P11 Kabat CDRH3 amino acid sequence
SEQ ID NO:6 sets forth the P11 Kabat CDRL1 amino acid sequence
SEQ ID NO:7 sets forth the P11 Kabat CDRL2 amino acid sequence
SEQ ID NO:8 sets forth the P11 Kabat CDRL3 amino acid sequence
SEQ ID NO:9 sets forth the amino acid sequence of mature heavy chain variable region of clone P17
SEQ ID NO: 10 sets forth the amino acid sequence of mature light chain variable region of clone P17
SEQ ID NO: 11 sets forth the P17 Kabat CDRH1 amino acid sequence
SEQ ID NO:12 sets forth the P17 Kabat CDRH2 amino acid sequence
SEQ ID NO:13 sets forth the P17 Kabat CDRH3 amino acid sequence
SEQ ID NO:14 sets forth the P17 Kabat CDRL1 amino acid sequence
SEQ ID NO:15 sets forth the P17 Kabat CDRL2 amino acid sequence
SEQ ID NO:16 sets forth the P17 Kabat CDRL3 amino acid sequence
SEQ ID NO: 17 sets forth the amino acid sequence of mature heavy chain variable region of clone P28
SEQ ID NO:18 sets forth the amino acid sequence of mature light chain variable region of clone P28
SEQ ID NO: 19 sets forth the P28 Kabat CDRH1 amino acid sequence
SEQ ID NO:20 sets forth the P28 Kabat CDRH2 amino acid sequence
SEQ ID NO:21 sets forth the P28 Kabat CDRH3 amino acid sequence
SEQ ID NO:22 sets forth the P28 Kabat CDRL1 amino acid sequence
SEQ ID NO:23 sets forth the P28 Kabat CDRL2 amino acid sequence
SEQ ID NO:24 sets forth the P28 Kabat CDRL3 amino acid sequence
SEQ ID NO:25 sets forth the amino acid sequence of mature heavy chain variable region of clone P130
SEQ ID NO:26 sets forth the amino acid sequence of mature light chain variable region of clone P130
SEQ ID NO:27 sets forth the P130 Kabat CDRH1 amino acid sequence
SEQ ID NO:28 sets forth the P130 Kabat CDRH2 amino acid sequence
SEQ ID NO:29 sets forth the P130 Kabat CDRH3 amino acid sequence
SEQ ID NO:30 sets forth the P130 Kabat CDRL1 amino acid sequence
SEQ ID NO:31 sets forth the P130 Kabat CDRL2 amino acid sequence
SEQ ID NO:32 sets forth the P130 Kabat CDRL3 amino acid sequence
SEQ ID NO:33 sets forth the amino acid sequence of mature heavy chain variable region of clone P240
SEQ ID NO:34 sets forth the amino acid sequence of mature light chain variable region of clone P240
SEQ ID NO:35 sets forth the P240 Kabat CDRH1 amino acid sequence
SEQ ID NO:36 sets forth the P240 Kabat CDRH2 amino acid sequence
SEQ ID NO:37 sets forth the P240 Kabat CDRH3 amino acid sequence
SEQ ID NO:38 sets forth the P240 Kabat CDRL1 amino acid sequence
SEQ ID NO:39 sets forth the P240 Kabat CDRL2 amino acid sequence
SEQ ID NO:40 sets forth the P240 Kabat CDRL3 amino acid sequence
SEQ ID NO:41 sets forth the amino acid sequence of mature heavy chain variable region of clone P1187
SEQ ID NO:42 sets forth the amino acid sequence of mature light chain variable region of clone P1187
SEQ ID NO:43 sets forth the P1187 Kabat CDRH1 amino acid sequence
SEQ ID NO:44 sets forth the P1187 Kabat CDRH2 amino acid sequence
SEQ ID NO:45 sets forth the P1187 Kabat CDRH3 amino acid sequence
SEQ ID NO:46 sets forth the P1187 Kabat CDRL1 amino acid sequence
SEQ ID NO:47 sets forth the P1187 Kabat CDRL2 amino acid sequence
SEQ ID NO:48 sets forth the P1187 Kabat CDRL3 amino acid sequence
SEQ ID NO:49 sets forth the amino acid sequence of mature heavy chain variable region of clone P1707
SEQ ID NO:50 sets forth the amino acid sequence of mature light chain variable region of clone P1707
SEQ ID NO:51 sets forth the P1707 Kabat CDRH1 amino acid sequence
SEQ ID NO:52 sets forth the P1707 Kabat CDRH2 amino acid sequence
SEQ ID NO:53 sets forth the P1707 Kabat CDRH3 amino acid sequence
SEQ ID NO: 54 sets forth the P1707 Kabat CDRL1 amino acid sequence
SEQ ID NO:55 sets forth the P1707 Kabat CDRL2 amino acid sequence
SEQ ID NO: 56 sets forth the P1707 Kabat CDRL3 amino acid sequence
SEQ ID NO: 57 sets forth the amino acid sequence of mature heavy chain variable region of clone P1955
SEQ ID NO:58 sets forth the amino acid sequence of mature light chain variable region of clone P1955
SEQ ID NO:59 sets forth the P1955 Kabat CDRH1 amino acid sequence
SEQ ID NO:60 sets forth the P1955 Kabat CDRH2 amino acid sequence
SEQ ID NO:61 sets forth the P1955 Kabat CDRH3 amino acid sequence
SEQ ID NO:62 sets forth the P1955 Kabat CDRL1 amino acid sequence
SEQ ID NO:63 sets forth the P1955 Kabat CDRL2 amino acid sequence
SEQ ID NO:64 sets forth the P1955 Kabat CDRL3 amino acid sequence
SEQ ID NO:65 sets forth the amino acid sequence of mature heavy chain variable region of clone P2388
SEQ ID NO:66 sets forth the amino acid sequence of mature light chain variable region of clone P2388
SEQ ID NO:67 sets forth the P2388 Kabat CDRH1 amino acid sequence
SEQ ID NO:68 sets forth the P2388 Kabat CDRH2 amino acid sequence
SEQ ID NO:69 sets forth the P2388 Kabat CDRH3 amino acid sequence
SEQ ID NO:70 sets forth the P2388 Kabat CDRL1 amino acid sequence
SEQ ID NO:71 sets forth the P2388 Kabat CDRL2 amino acid sequence
SEQ ID NO:72 sets forth the P2388 Kabat CDRL3 amino acid sequence
SEQ ID NO: 73 sets forth the amino acid sequence of mature heavy chain variable region of clone P2653
SEQ ID NO:74 sets forth the amino acid sequence of mature light chain variable region of clone P2653
SEQ ID NO:75 sets forth the P2653 Kabat CDRH1 amino acid sequence
SEQ ID NO:76 sets forth the P2653 Kabat CDRH2 amino acid sequence
SEQ ID NO:77 sets forth the P2653 Kabat CDRH3 amino acid sequence
SEQ ID NO:78 sets forth the P2653 Kabat CDRL1 amino acid sequence
SEQ ID NO:79 sets forth the P2653 Kabat CDRL2 amino acid sequence
SEQ ID NO:80 sets forth the P2653 Kabat CDRL3 amino acid sequence
SEQ ID NO:81 sets forth the amino acid sequence of mature heavy chain variable region of clone P2861
SEQ ID NO:82 sets forth the amino acid sequence of mature light chain variable region of clone P2861
SEQ ID NO:83 sets forth the P2861 Kabat CDRH1 amino acid sequence
SEQ ID NO:84 sets forth the P2861 Kabat CDRH2 amino acid sequence
SEQ ID NO:85 sets forth the P2861 Kabat CDRH3 amino acid sequence
SEQ ID NO:86 sets forth the P2861 Kabat CDRL1 amino acid sequence
SEQ ID NO:87 sets forth the P2861 Kabat CDRL2 amino acid sequence
SEQ ID NO:88 sets forth the P2861 Kabat CDRL3 amino acid sequence
SEQ ID NO:89 sets forth the amino acid sequence of mature heavy chain variable region of clone P3208
SEQ ID NO:90 sets forth the amino acid sequence of mature light chain variable region of clone P3208
SEQ ID NO:91 sets forth the P3208 Kabat CDRH1 amino acid sequence
SEQ ID NO:92 sets forth the P3208 Kabat CDRH2 amino acid sequence
SEQ ID NO:93 sets forth the P3208 Kabat CDRH3 amino acid sequence
SEQ ID NO:94 sets forth the P3208 Kabat CDRL1 amino acid sequence
SEQ ID NO: 95 sets forth the P3208 Kabat CDRL2 amino acid sequence
SEQ ID NO: 96 sets forth the P3208 Kabat CDRL3 amino acid sequence
SEQ ID NO: 97 sets forth the amino acid sequence of mature heavy chain variable region of clone N277
SEQ ID NO:98 sets forth the amino acid sequence of mature light chain variable region of clone N277
SEQ ID NO:99 sets forth the N277 Kabat CDRH11 amino acid sequence
SEQ ID NO:100 sets forth the N277 Kabat CDRH2 amino acid sequence
SEQ ID NO:101 sets forth the N277 Kabat CDRH3 amino acid sequence
SEQ ID NO:102 sets forth the N277 Kabat CDRL1 amino acid sequence
SEQ ID NO:103 sets forth the N277 Kabat CDRL2 amino acid sequence
SEQ ID NO:104 sets forth the N277 Kabat CDRL3 amino acid sequence
SEQ ID NO: 105 sets forth the amino acid sequence of mature heavy chain variable region of clone N350
SEQ ID NO: 106 sets forth the amino acid sequence of mature light chain variable region of clone N350
SEQ ID NO: 107 sets forth the N350 Kabat CDRH1 amino acid sequence
SEQ ID NO: 108 sets forth the N350 Kabat CDRH2 amino acid sequence
SEQ ID NO: 109 sets forth the N350 Kabat CDRH3 amino acid sequence
SEQ ID NO:110 sets forth the N350 Kabat CDRL1 amino acid sequence
SEQ ID NO: 111 sets forth the N350 Kabat CDRL2 amino acid sequence
SEQ ID NO:112 sets forth the N350 Kabat CDRL3 amino acid sequence
SEQ ID NO: 113 sets forth the amino acid sequence of mature heavy chain variable region of clone N484
SEQ ID NO:114 sets forth the amino acid sequence of mature light chain variable region of clone N484
SEQ ID NO: 115 sets forth the N484 Kabat CDRH1 amino acid sequence
SEQ ID NO: 116 sets forth the N484 Kabat CDRH2 amino acid sequence
SEQ ID NO:117 sets forth the N484 Kabat CDRH3 amino acid sequence
SEQ ID NO: 118 sets forth the N484 Kabat CDRL1 amino acid sequence
SEQ ID NO: 119 sets forth the N484 Kabat CDRL2 amino acid sequence
SEQ ID NO:120 sets forth the N484 Kabat CDRL3 amino acid sequence
SEQ ID NO: 121 sets forth the amino acid sequence of mature heavy chain variable region of clone N2282
SEQ ID NO: 122 sets forth the amino acid sequence of mature light chain variable region of clone N2282
SEQ ID NO: 123 sets forth the N2282 Kabat CDRH1 amino acid sequence
SEQ ID NO: 124 sets forth the N2282 Kabat CDRH2 amino acid sequence
SEQ ID NO: 125 sets forth the N2282 Kabat CDRH3 amino acid sequence
SEQ ID NO: 126 sets forth the N2282 Kabat CDRL1 amino acid sequence
SEQ ID NO: 127 sets forth the N2282 Kabat CDRL2 amino acid sequence
SEQ ID NO: 128 sets forth the N2282 Kabat CDRL3 amino acid sequence
SEQ ID NO: 129 sets forth the amino acid sequence of mature heavy chain variable region of clone N2430
SEQ ID NO: 130 sets forth the amino acid sequence of mature light chain variable region of clone N2430
SEQ ID NO: 131 sets forth the N2430 Kabat CDRH1 amino acid sequence
SEQ ID NO: 132 sets forth the N2430 Kabat CDRH2 amino acid sequence
SEQ ID NO: 133 sets forth the N2430 Kabat CDRH3 amino acid sequence
SEQ ID NO: 134 sets forth the N2430 Kabat CDRL1 amino acid sequence
SEQ ID NO:135 sets forth the N2430 Kabat CDRL2 amino acid sequence
SEQ ID NO:136 sets forth the N2430 Kabat CDRL3 amino acid sequence
SEQ ID NO: 137 sets forth the amino acid sequence of mature heavy chain variable region of clone E2810
SEQ ID NO: 138 sets forth the amino acid sequence of mature light chain variable region of clone E2810
SEQ ID NO: 139 sets forth the E2810 Kabat CDRH1 amino acid sequence
SEQ ID NO: 140 sets forth the E2810 Kabat CDRH2 amino acid sequence
SEQ ID NO: 141 sets forth the E2810 Kabat CDRH3 amino acid sequence
SEQ ID NO: 142 sets forth the E2810 Kabat CDRL1 amino acid sequence
SEQ ID NO: 143 sets forth the E2810 Kabat CDRL2 amino acid sequence
SEQ ID NO: 144 sets forth the E2810 Kabat CDRL3 amino acid sequence
SEQ ID NO: 145 sets forth the amino acid sequence of Human PTPRG UniProt No. P23470
SEQ ID NO: 146 sets forth the amino acid sequence of Human Nectin-2 UniProt Q92692-1
SEQ ID NO: 147 sets forth the amino acid sequence of human endoglin UniProt P17813-1
SEQ ID NO:148 sets forth the amino acid sequence of mouse PTPRG UniProt No. Q05909.

### Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “antibody” also synonymously called “immunoglobulins” (Ig), or “antigen-binding fragment” refers to polypeptide chain(s) which exhibit a specific monovalent, bivalent or polyvalent binding to a given antigen, epitope or epitopes. Unless otherwise noted, antibodies or antigen-binding fragments used in the invention can have sequences derived from any vertebrate species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antigen-binding polypeptide fragments, bispecific and other designer antibodies that are described below or well known in the art (see, e.g., Serafini, J Nucl. Med. 34:533-6, 1993), T-cell engaging bispecific antibodies (T-biAbs), NK-cell engaging bispecific antibodies (NK-biAbs), antibodies conjugated to other molecules, such as antibody drug conjugates, radioimmunoconjugates, antibody based binding proteins and chimeric antigen receptors including VH and VL regions.

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (V_{H}) and a heavy chain constant region. The heavy chain constant region of most IgG isotypes (subclasses) is comprised of three domains, C_{H1}, C_{H2} and C_{H3}, some IgG isotypes, like IgM or IgE comprise a fourth constant region domain, C_{H4}. Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991) or IMGT (imgt.org).

An “antigen binding site” or “antigen-binding region” of an immunoglobulin of the present invention typically contains six complementarity determining regions (CDRs) within each variable domain, and which contribute in varying degrees to the affinity of the binding site for antigen. In each variable domain there are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences and/or structural information from antibody/antigen complexes. Also included within the scope of the invention are functional antigen binding sites comprised of fewer CDRs (i.e., where binding specificity is determined by three, four or five CDRs). Less than a complete set of 6 CDRs can be sufficient for binding to some binding targets. Thus, in some instances, the CDRs of a V_{H} or a V_{L} domain alone will be sufficient. Furthermore, certain antibodies, in particular rabbit antibodies, might have non-CDR-associated binding sites for an antigen. Such binding sites are specifically included within the present definition.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which HCDR1 is a composite of Chothia and Kabat CDRs; the AbM definition used by Oxford Molecular's antibody modelling software; and, the contact definition of Martin et al (bioinfo.org.uk/abs), and IMGT definition (imgt.org/IMGTScientificChart/Numbering/IMGTnumberingCDR_VK.html) (see Table 1). Kabat provides a widely used numbering convention (Kabat numbering) in which corresponding residues between different heavy chains or between different light chains are assigned the same number. When an antibody is said to comprise CDRs by a certain definition of CDRs (e.g., IMGT) that definition specifies the minimum number of CDR residues present in the antibody (i.e., the IMGT CDRs). It does not exclude that other residues falling within another conventional CDR definition but outside the specified definition are also present.

TABLE 1
Conventional Definitions of CDRs Using Kabat Numbering

| | | | Composite of | | | |
|---|---|---|---|---|---|---|
| Loop | Kabat | Chothia | Chothia & Kabat | AbM | Contact | IMGT |
| L1 | L24--L34 | L24--L34 | L24--L34 | L24--L34 | L30--L36 | L27-L32 |
| L2 | L50--L56 | L50--L56 | L50--L56 | L50--L56 | L46--L55 | L50-L52 |
| L3 | L89--L97 | L89--L97 | L89--L97 | L89--L97 | L89--L96 | L89-L97 |
| H1 | H31--H35B | H26-- | H26--H35B* | H26--H35B | H30--H35B | H26-H33 |
| | | H32 . . . H34* | | | | |
| H2 | H50--H65 | H52--H56 | H50--H65 | H50--H58 | H47--H58 | H51-H56 |
| H3 | H95--H102 | H95--H102 | H95--H102 | H95--H102 | H93--H101 | H93-H102 |

*HCDR1 by Chothia can end at H32, H33, or H34 (depending on the length of the loop). This is because the Kabat numbering scheme places insertions of extra residues at 35A and 35B, whereas Chothia numbering places them at 31A and 31B. If neither H35A nor H35B (Kabat numbering) is present, the Chothia HCDR1 loop ends at H32. If only H35A is present, it ends at H33. If both H35A and H35B are present, it ends at H34.

An “antibody-based binding protein”, as used herein, may represent any protein that contains at least one antibody-derived V_{H}, V_{L}, in the context of other non-immunoglobulin, or non-antibody derived components. Such antibody-based proteins include, but are not limited to (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments (antigen-binding fragments).

“Binding affinity” is generally expressed in terms of equilibrium association or dissociation constants (K_{A} or K_{D}, respectively), which are in turn reciprocal ratios of dissociation and association rate constants (koff and kon, respectively). Thus, equivalent affinities may correspond to different rate constants, so long as the ratio of the rate constants remains the same. The binding affinity of an antibody is usually be expressed as the K_{D} of a monovalent fragment (e.g. a F_{ab} fragment) of the antibody, with K_{D} values in the single-digit nanomolar range or below (subnanomolar or picomolar) being considered as very high and of therapeutic and diagnostic relevance.

As used herein, the term “binding specificity” refers to the selective affinity of one molecule for another such as the binding of antibodies to antigens (or an epitope or antigenic determinant thereof), receptors to ligands, and enzymes to substrates. Thus, all monoclonal antibodies that bind to a particular antigenic determinant of an entity (e.g., a specific epitope of an acute myeloid leukemia cell surface antigen, including any of PTPRG, Nectin-2, or endoglin) are deemed to have the same binding specificity for that entity.

The term “Antibody Drug Conjugate”, or “ADC” refers to an antibody to which a therapeutically active substance or an active pharmaceutical ingredient (API) has been covalently linked, such that the therapeutically active substance or an active pharmaceutical ingredient (API) can be targeted to the binding target of the antibody to exhibit its pharmacologic function. Linkage can be direct via a chemical bond or indirect through a linker. Some linkers are cleavable under physiological conditions, whereas other linkers are not. The therapeutically active substance or an active pharmaceutical ingredient can be a cellular toxin that is able to effect killing of the cells targeted by the ADCs, preferably malignant or cancer cells. The covalent attachment of a therapeutically active substance, an active pharmaceutical ingredient or a cellular toxin can be performed in a non-site specific manner using standard chemical linkers that couple payloads to lysine or cysteine residues, or, preferably the conjugation is performed in a site-specific manner, that allows full control of conjugation site and drug to antibody ratio (DAR) of the ADC to be generated.

The term “radioimmunoconjugate”, or “RIC” refers to an antibody to which a radionuclide has been attached, such that the radionuclide can be targeted to the binding target of the antibody to deliver cytotoxic radiation to a target cell, for example a cancer cell. In some embodiments, the radionuclide is directly attached to the antibody via a covalent bond or is attached to the antibody via a chelation complex.

The terms “T-cell engaging bispecific antibody (T-biAb)” and “NK-cell engaging bispecific antibody (NK-biAb)” refer to a bispecific antibody that simultaneously binds to an activating receptor on T cells (e.g., CD3) and NK cells (e.g., CD16), respectively, and a target-associated antigen on a target cell, activates T/NK cells, and redirects T/NK cells' cytotoxicity against the target cell.

The term “conservatively modified variant” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or phage), combining agents and cells, or combining two populations of different cells. Contacting can occur in vitro, e.g., mixing an antibody and a cell or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject, e.g., by administering an agent to a subject for delivery the agent to a target cell.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Natl. Acad. Sci. USA 85:2444, 1988; by computerized implementations of these algorithms (GAP, BESTFIT, FASTA, and TFASTA in the Wisconsin Genetics Software Package, Genetics Computer Group, Madison, WI); or by manual alignment and visual inspection (see, e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nucleic Acids Res. 25:3389-3402, 1977; and Altschul et al., J. Mol. Biol. 215:403-410, 1990, respectively.

The term “subject” refers to human and non-human animals (especially non-human mammals). The term “subject” is used herein, for example, in connection with therapeutic and diagnostic methods, to refer to human or animal subjects. Animal subjects include, but are not limited to, animal models, such as, mammalian models of conditions or disorders associated a hematologic malignancy such as acute myeloid leukemia (AML). Other specific examples of non-human subjects include, e.g., cows, horses, sheep, pigs, cats, dogs, mice, rats, rabbits, guinea pigs, monkeys.

Artificial T cell receptors (also known as chimeric T cell receptors, chimeric immunoreceptors, chimeric antigen receptors (CARs) or T-bodies) are engineered receptors, which graft an arbitrary specificity onto an immune effector cell. Typically, these receptors are used to graft the specificity of a monoclonal antibody onto a T cell; with transfer of their coding sequence facilitated by retroviral or lentiviral vectors or by transposons. CAR-engineered T cells (also abbreviated CAR-T cells) are genetically engineered T cells armed with chimeric receptors whose extracellular recognition unit is comprised of an antibody-derived recognition domain and whose intracellular region is derived from lymphocyte stimulating moiety(ies). The structure of the prototypic CAR is modular, designed to accommodate various functional domains and thereby to enable choice of specificity and controlled activation of T cells. The preferred antibody-derived recognition unit is a single chain variable fragment (scFv) that combines the specificity and binding residues of both the heavy and light chain variable regions of a monoclonal antibody. The most common lymphocyte activation moieties include a T-cell costimulatory (e.g. CD28) domain in tandem with a T-cell triggering (e.g. CD3zeta) moiety. By arming effector lymphocytes (such as T cells and natural killer cells) with such chimeric receptors, the engineered cell is redirected with a predefined specificity to any desired target antigen, in a non-HLA restricted manner. CAR constructs are introduced ex vivo into T cells from peripheral lymphocytes of a given patient using retroviral or lentiviral vectors or transposons. Following infusion of the resulting CAR-engineered T cells back into the patient, they traffic, reach their target site, and upon interaction with their target cell or tissue, they undergo activation and perform their predefined effector function. Therapeutic targets for the CAR approach include cancer and HIV-infected cells, or autoimmune effector cells. CAR-engineered NK cells are conceptually and architecturally related to CAR-engineered T cells.

The terms “treat,” “treating,” “treatment,” and “therapeutically effective” used herein do not necessarily imply 100% or complete treatment. Rather, there are varying degrees of treatment recognized by one of ordinary skill in the art as having a potential benefit or therapeutic effect. In this respect, the inventive method can provide any amount of any level of treatment. Furthermore, the treatment provided by the inventive method can include the treatment of one or more conditions or symptoms of the disease being treated.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

### DETAILED DESCRIPTION

### I. Overview

In one aspect, the invention provides antibodies that specifically bind PTPRG, Nectin-2, and endoglin. Exemplary antibodies were identified from highly diverse phage-display libraries of non-immunized rabbits by differential selection on acute myeloid leukemia cells versus healthy donor peripheral blood mononuclear cells (PBMCs). By this strategy antibodies for PTPRG, Nectin-2, and endoglin of high quality have been identified. Furthermore, the invention provides chimeric full-length antibodies of the rabbit variable domains fused to the constant region domains of human IgG₁ antibodies. The invention also provides humanized or chimeric full-length antibodies of the rabbit CDRs grafted into human variable region frameworks or rabbit variable region frameworks, in either case fused to the constant region domains of human IgG₁ antibodies.

The present invention provides monoclonal antibodies and related antibody-based binding proteins and antibody fragments (antigen-binding fragments) thereof that specifically recognize acute myeloid leukemia cell surface antigens PTPRG, Nectin-2, and endoglin. Exemplary antibodies of the invention are P11, P17, P28, P130, P240, P1187, P1707, P1955, P2388, P2653, P2861, P3208, N277, N350, N484, N2282, N2430, and E2810. Exemplary antibodies of the invention binding PTPRG are P11, P17, P28, P130, P240, P1187, P1707, P1955, P2388, P2653, P2861, and P3208. Exemplary antibodies of the invention binding Nectin-2 are N277, N350, N484, N2282, and N2430. An exemplary antibody of the invention binding endoglin is E2810.

The invention also provides antibody drug conjugates (ADCs), radioimmunoconjugates (RICs), T-cell engaging bispecific antibodies (T-biAbs), NK-cell engaging bispecific antibodies (NK-biAbs), chimeric antigen receptors (CARs) derived from the antibodies binding PTPRG, Nectin-2, and endoglin described herein. Further provided in the invention are methods of using these antibody agents and related compositions in therapeutic and diagnostic applications for diseases and conditions with associated abnormal or elevated PTPRG, Nectin-2, and endoglin expression, e.g., hematologic malignancies including acute myeloid leukemia (AML).

### II. Antibody Discovery by Phage Display

Using a phage display selection strategy that combines previously published ‘Fab biotinylation and capture (FBC)’ method (Wilson, H. D. et al., J Mol Biol. 2018 Oct. 19; 430(21):4387-4400) with next-generation sequencing (NGS), the inventors mined for rabbit monoclonal antibodies (mAbs) in chimeric rabbit/human Fab format that selectively bind to acute myeloid leukemia (AML) cell lines but not to healthy peripheral blood mononuclear cells (PBMC). The objective was to identify candidate cell surface antigens for antibody-based therapy of AML. This antibody-driven antigen discovery campaign led to the discovery of PTPRG, Nectin-2, and CD105 (also known as endoglin). The corresponding rabbit anti-human PTPRG (12 mAbs), Nectin-2 (5 mAbs), and endoglin (CD105; 1 mAb) mAbs are reported here.

PTPRG, Nectin-2, or endoglin can each be targeted on its own. In other embodiments, any one, two, or three of PTPRG, Nectin-2, and endoglin can targeted in combination with each other, and/or with other cell surface antigens (AND gate) for next-generation CAR-Ts and other “logic biologics”.

Antibody-based biologics are well established treatments of cancer. Antibody drug discovery campaigns are typically directed at a single target of interest, which inherently limits the possibility of uncovering novel antibody specificities or functionalities. Here, we present a target unbiased approach for anti-tumor antibody discovery that relies on generating antibodies against native cancer cell surfaces via phage display. Applying this method to acute myeloid leukemia (AML) cells yielded a diverse panel of 28 antibodies with unique sequences and diverse reactivities. To uncover the identity of the cognate targets recognized by this panel, representative mAbs from each of 3 unique reactivity clusters were used in a new multi-omic target deconvolution approach which leverages proteomic and comparative transcriptomic analyses. From this, three distinct targets were identified: PTPRG, Nectin-2, and endoglin.

In our previous work, we have discovered novel therapeutic targets from target-agnostic selections of antibody libraries using phage display[10]. However, these selections typically converged on a single target, which subsequently took several years to deconvolute [19]. Recently, our lab developed a more efficient platform for selecting phage-display libraries on human cells that eliminates the ~95% of bald phage, termed Fab-phage biotinylation and capture (FBC) [20]. The FBC method enzymatically biotinylates the Fab, allowing for the removal of bald (Fab- and biotin-less) phage between the elution and re-amplification steps, increasing the panning efficiency. However, the implementation of FBC for target-agnostic WCP is limited by the low-throughput process of colony screening, which rarely exceeds 10² or 10³ clones, while selected libraries can have ~10⁴⁻⁷ unique mAbs. Thus low-abundance clones of interest are potentially missed. During WCP, this complication can be exacerbated by the outgrowth of immunodominant mAbs that bind irrelevant epitopes. A common approach to this problem is negative selection or depletion on non-target cells or vesicles, but this may eliminate mAbs against targets with low basal expression on the non-target cells, even if it is highly over-expressed on the target cells [6].⁶

New developments in sequencing technologies have improved the depth of antibody discovery using phage display and thus enabled a more comprehensive assessment of output antibody diversity [21-23]. Also, by directly removing sequence redundancy, it reduces the degree of back-end screening required. Few reports exist of next generation sequencing (NGS) analyses of WCP outputs, presumably due to the technical challenge of high background phage, which leads to inordinately large and complex phage outputs [24-26]. Furthermore bald phage depletion has not been used to enhance the selection efficiency. We hypothesize that bald phage depletion augments NGS-based analyses of phage antibody outputs. By reducing background bald phage propagation, FBC panning favorably compresses output library diversity and increases the abundance of cell surface binding mAbs, which will enhance the coverage of libraries provided by NGS, and in turn the identification of rare clones.

A streamlined methodology for antibody drug and target discovery using WCP was used, termed FBC-seq, that hinges on the differential selection of Fab-phage libraries on target versus non-target cell types and determines the enrichment of mAbs captured by the target cell population using NGS. The FBC approach was employed to differentially select on AML cells versus healthy donor peripheral blood mononuclear cells (PBMCs). Diverse mAb targets were identified from these orphan mAbs, and for this a dual proteomics and transcriptomics approach was found highly effective.

### III. Protein Targets

PTPRG is a member of the Protein Tyrosine Phosphatase (PTP) family. The mature protein comprises 1426 amino acids of which approximately half form its extracellular domain (ECD) followed by a single transmembrane region and two intracellular phosphatase domains. The ECD contains a carbonic anhydrase (CAH)-like domain and a fibronectin type III domain. PTPRG can form homodimers. An exemplary human PTPRG amino acid sequence is UniProt P23470-1, provided as SEQ ID NO:145. SEQ ID NO:145 includes the 19 amino acid N-terminal signal sequence. An exemplary mouse PTPRG amino acid sequence is UniProt No. Q05909, provided as SEQ ID NO:148. SEQ ID NO:148 includes a 19 amino acid N-terminal signal sequence.

Reference to PTPRG includes known natural variations which are listed in the Swiss-Prot database and permutations thereof, as well as mutations associated with pathologies. Additionally, reference to PTPRG includes PTPRG with known post-translational modifications. Examples of known post-translational modifications are listed in the UniProtKB/Swiss-Prot database. Unless otherwise apparent from context, reference to PTPRG, or its fragments includes the natural human amino acid sequences including isoforms, mutants, and allelic variants thereof. Unless otherwise apparent from the context, reference to PTPRG means a natural human form of PTPRG including all isoforms, including soluble forms, irrespective of whether posttranslational modification (e.g., phosphorylation, glycation, or acetylation) is present.

Nectin-2, (also known as Herpesvirus entry mediator B; HveB, Nectin cell adhesion molecule 2, Poliovirus receptor-related protein 2, and CD112) is a single-pass transmembrane glycoprotein that functions as a Ca++-independent cell adhesion molecule. The mature protein comprises 507 amino acids, including an immunoglobulin (Ig)-like V-type domain, an Ig-like C2-type 1 domain, an Ig-like C2-type 2 domain, a transmembrane domain, and a cytoplasmic domain. Nectin-2 can form homodimers. An exemplary human Nectin-2 amino acid sequence is UniProt Q92692-1, provided as SEQ ID NO:146. SEQ ID NO: 146 includes the 31 amino acid N-terminal signal sequence.

Reference to Nectin-2 includes known natural variations which are listed in the Swiss-Prot database and permutations thereof, as well as mutations associated with pathologies. Additionally, reference to Nectin-2 includes Nectin-2 with known post-translational modifications. Examples of known post-translational modifications are listed in the UniProtKB/Swiss-Prot database. Unless otherwise apparent from context, reference to Nectin-2, or its fragments includes the natural human amino acid sequences including isoforms, mutants, and allelic variants thereof. Unless otherwise apparent from the context, reference to Nectin-2 means a natural human form of Nectin-2 including all isoforms, including soluble forms, irrespective of whether posttranslational modification (e.g., phosphorylation, glycation, or acetylation) is present.

Endoglin (also known as CD105) is a single-pass transmembrane glycoprotein that plays a role in regulation of angiogenesis. The mature protein comprises 633 amino acids including an extracellular domain, a transmembrane domain, and a cytoplasmic domain. Endoglin can form homodimers. An exemplary human endoglin amino acid sequence is UniProt P17813-1, provided as SEQ ID NO: 147, includes the 25 amino acid N-terminal signal sequence.

Reference to endoglin includes known natural variations which are listed in the Swiss-Prot database and permutations thereof, as well as mutations associated with pathologies. Additionally, reference to endoglin includes endoglin with known post-translational modifications. Examples of known post-translational modifications are listed in the UniProtKB/Swiss-Prot database. Unless otherwise apparent from context, reference to endoglin, or its fragments includes the natural human amino acid sequences including isoforms, mutants, and allelic variants thereof. Unless otherwise apparent from the context, reference to endoglin means a natural human form of endoglin including all isoforms, including soluble forms, irrespective of whether posttranslational modification (e.g., phosphorylation, glycation, or acetylation) is present.

### IV. Antibodies Specifically Binding to an Acute Myeloid Leukemia Cell Surface Antigen for Example PTPRG, Nectin-2, or Endoglin

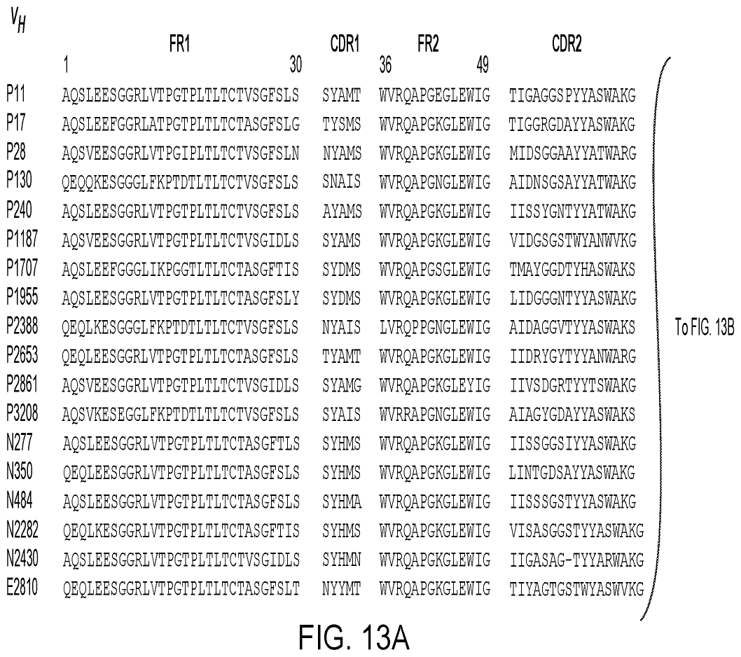

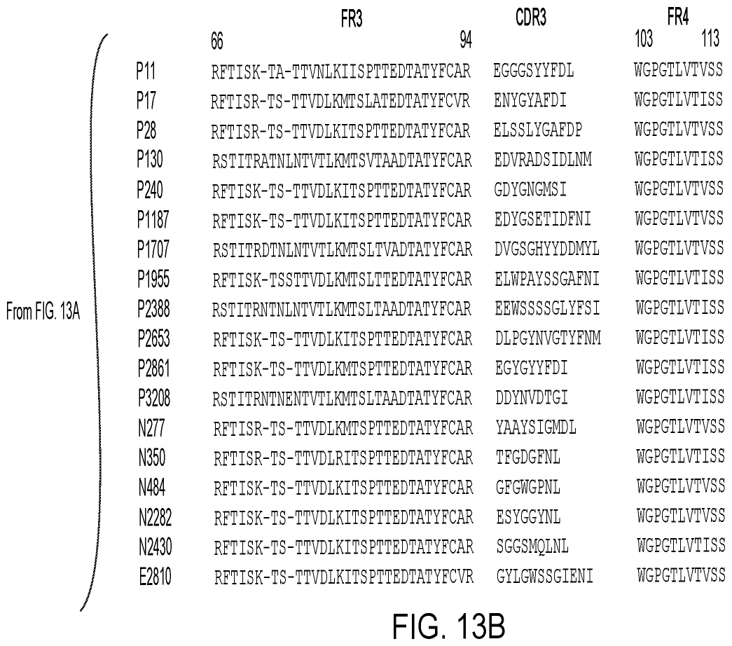

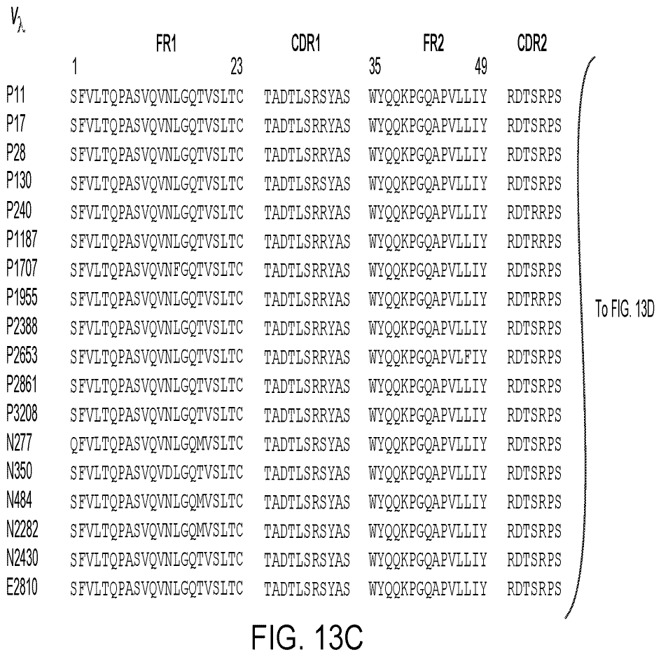

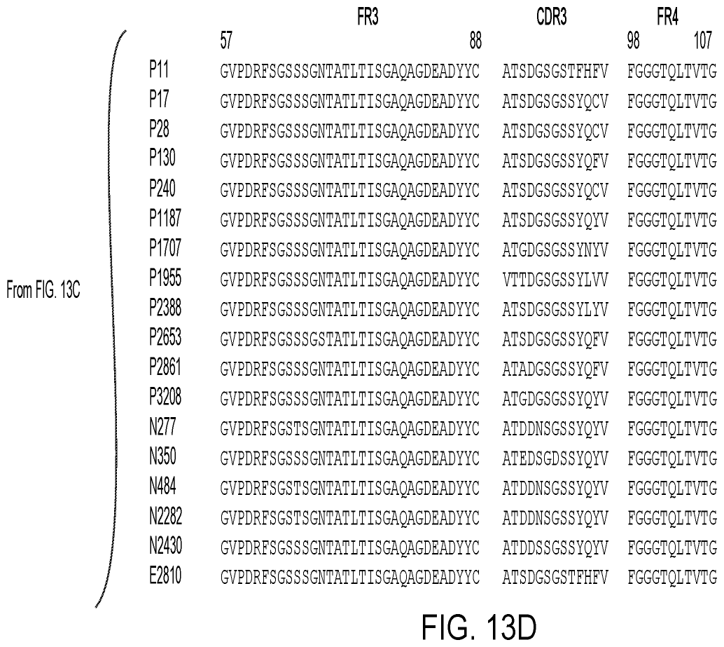

In one aspect, the invention provides antibodies, antibody-based binding proteins, antibody fragments (also termed “antigen-binding fragments”) thereof, ADCs, RICs, T-biAbs, NK-biAbs, or CARs that specifically bind to human PTPRG, Nectin-2, or endoglin with the same binding specificity as that of anti-PTPRG, Nectin-2, or endoglin antibodies exemplified herein (FIG. 13). Antibodies of the invention include intact antibodies (e.g., IgG1 antibodies exemplified herein), antibody fragments (e.g., Fab antibodies exemplified herein), antibody-based binding proteins, antibody fragments thereof, ADCs radioimmunoconjugates (RICs), T- or NK-cell engaging bispecific antibodies (T-biAbs or NK-biAbs), or CARs which contain the antigen-binding portions of an intact antibody that retain capacity to bind the cognate antigen, PTPRG, Nectin-2, or endoglin. Examples of such antibody fragments include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} or V_{L} domain (see, e.g., Ward et al., Nature 341:544-546, 1989); and (vii) an isolated complementarity determining region (CDR) as a linear or cyclic peptide. Examples of antibody-based binding proteins are polypeptides in which the binding domains of the antibodies are combined with other polypeptides or polypeptide domains, e.g. alternative molecular scaffolds, Fc-regions, other functional or binding domains of other polypeptides or antibodies resulting in molecules with addition binding properties, e.g. bi- or multispecific proteins or antibodies. Such polypeptides can create an arrangement of binding or functional domains normally not found in naturally occurring antibodies or antibody fragments.

Antibodies of the invention also encompass antibody fragments, like single chain antibodies. The term “single chain antibody” refers to a polypeptide comprising a V_{H} domain and a V_{L} domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the V_{L} and V_{H} domains of the Fv fragment which are coded for by separate genes, a scFv has the two domains joined (e.g., via recombinant methods) by a synthetic linker. This enables them to be made as a single protein chain in which the V_{L} and V_{H} regions pair to form monovalent molecules.

Antibodies of the present invention also encompass single domain antigen-binding units, which have a camelid scaffold. Animals in the camelid family include camels, llamas, and alpacas. Camelids produce functional antibodies devoid of light chains. The heavy chain variable (V_{H}) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies.

The various antibodies, antibody-based binding proteins, and antibody fragments thereof described herein can be produced by enzymatic or chemical modification of the intact antibodies, or synthesized de novo using recombinant DNA methodologies, or identified using phage display libraries. Methods for generating these antibodies, antibody-based binding proteins, and antibody fragments thereof are all well known in the art. For example, single chain antibodies can be identified using phage display libraries or ribosome display libraries, gene shuffled libraries (see, e.g., McCafferty et al., Nature 348:552-554, 1990; and U.S. Pat. No. 4,946,778). In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and Plückthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cai and Garen, Proc. Natl. Acad. Sci. USA 93:6280-85, 1996. Camelid single domain antibodies can be produced using methods well known in the art, e.g., Dumoulin et al., Nat. Struct. Biol. 11:500-515, 2002; Ghahroudi et al., FEBS Letters 414:521-526, 1997; and Bond et al., J. Mol. Biol. 332:643-55, 2003. Other types of antigen-binding fragments (e.g., Fab, F(ab′)2 or Fd fragments) can also be readily produced with routinely practiced immunology methods. See, e.g., Harlow & Lane, Using Antibodies, A Laboratory Manual, Cold Spring Harbor Laboratory Press, Cold Spring Harbor, New York, 1998.

Other antibodies can be obtained by mutagenesis of cDNA encoding the heavy and light chains of an exemplary antibody, such as P11, P17, P28, P130, P240, P1187, P1707, P1955, P2388, P2653, P2861, P3208, N277, N350, N484, N2282, N2430, or E2810. Monoclonal antibodies that are at least 70%, 80%, 90%, 95%, 96%, 97%, 98%, or 99% identical to P11, P17, P28, P130, P240, P1187, P1707, P1955, P2388, P2653, P2861, P3208, N277, N350, N484, N2282, N2430, or E2810 in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention. Monoclonal antibodies having at least one or all six CDR(s) as defined by any conventional definition, e.g., Kabat or IMGT, that are 90%, 95%, 99% or 100% identical to corresponding CDRs of P11, P17, P28, P130, P240, P1187, P1707, P1955, P2388, P2653, P2861, P3208, N277, N350, N484, N2282, N2430, or E2810 are also included.

In some embodiments, the invention provides antibodies, antibody-based binding proteins or antibody fragments thereof that are conservatively modified variants of the anti-PTPRG, anti-Nectin-2, or anti-endoglin antibodies exemplified herein. Typically, the variable regions of these variants have an amino acid sequence that is identical to one of these exemplified sequences except for conservative substitutions at one or more amino acid residues. The invention also provides antibodies having some or all (e.g., 1, 2, 3, 4, 5, and 6) CDRs entirely or substantially from P11, P17, P28, P130, P240, P1187, P1707, P1955, P2388, P2653, P2861, P3208, N277, N350, N484, N2282, N2430, or E2810. Such antibodies can include a heavy chain variable region that has at least two, and usually all three, CDRs entirely or substantially from the heavy chain variable region of P11, P17, P28, P130, P240, P1187, P1707, P1955, P2388, P2653, P2861, P3208, N277, N350, N484, N2282, N2430, or E2810 and/or a light chain variable region having at least two, and usually all three, CDRs entirely or substantially from the light chain variable region of P11, P17, P28, P130, P240, P1187, P1707, P1955, P2388, P2653, P2861, P3208, N277, N350, N484, N2282, N2430, or E2810. The antibodies can include both heavy and light chains. A CDR is substantially from a corresponding P11, P17, P28, P130, P240, P1187, P1707, P1955, P2388, P2653, P2861, P3208, N277, N350, N484, N2282, N2430, or E2810 CDR when it contains no more than 4, 3, 2, or 1 substitutions, insertions, or deletions, except that HCDR2 (when defined by Kabat) can have no more than 6, 5, 4, 3, 2, or 1 substitutions, insertions, or deletions. Such antibodies can have at least 70%, 80%, 90%, 95%, 96%, 97%, 98%, or 99% identity to P11, P17, P28, P130, P240, P1187, P1707, P1955, P2388, P2653, P2861, P3208, N277, N350, N484, N2282, N2430, or E2810 in the amino acid sequence of the mature heavy and/or light chain variable regions and maintain their functional properties, and/or differ from P11, P17, P28, P130, P240, P1187, P1707, P1955, P2388, P2653, P2861, P3208, N277, N350, N484, N2282, N2430, or E2810 by a small number of functionally inconsequential amino acid substitutions (e.g., conservative substitutions), deletions, or insertions.

In some embodiments, the antibodies of the invention comprise heavy chain CDR1, CDR2 and CDR3 sequences and light chain CDR1, CDR2 and CDR3 sequences substantially identical to those of antibodies P11, P17, P28, P130, P240, P1187, P1707, P1955, P2388, P2653, P2861, P3208, N277, N350, N484, N2282, N2430, or E2810. Some antibodies comprise heavy chain CDR1, CDR2 and CDR3 sequences and light chain CDR1, CDR2 and CDR3 sequences identical to those of antibodies P11, P17, P28, P130, P240, P1187, P1707, P1955, P2388, P2653, P2861, P3208, N277, N350, N484, N2282, N2430, or E2810. In some embodiments, the antibodies of the invention have their heavy chain CDR1, CDR2 and CDR3 sequences and their light chain CDR1, CDR2 and CDR3 sequences that are substantially identical to that of the antibodies shown in FIG. 13. The light chain and heavy chain Kabat CDR sequences of the exemplified antibodies are all indicated in the figure. In some of these embodiments, the antibodies have (1) heavy chain CDR1-3 sequences that are substantially identical to SEQ ID NOs:3-5, respectively; and light chain CDR1-3 sequences that are substantially identical to SEQ ID NOs:6-8, respectively; (2) heavy chain CDR1-3 sequences that are substantially identical to SEQ ID NOs:11-13, respectively; and light chain CDR1-3 sequences that are substantially identical to SEQ ID NOs:14-16, respectively; (3) heavy chain CDR1-3 sequences that are substantially identical to SEQ ID NOs:19-21, respectively; and light chain CDR1-3 sequences that are substantially identical to SEQ ID NOs:22-24, respectively; (4) heavy chain CDR1-3 sequences that are substantially identical to SEQ ID NOs:27-29, respectively; and light chain CDR1-3 sequences that are substantially identical to SEQ ID NOs:30-32, respectively; (5) heavy chain CDR1-3 sequences that are substantially identical to SEQ ID NOs:35-37, respectively; and light chain CDR1-3 sequences that are substantially identical to SEQ ID NOs:38-40, respectively; (6) heavy chain CDR1-3 sequences that are substantially identical to SEQ ID NOs:43-45, respectively; and light chain CDR1-3 sequences that are substantially identical to SEQ ID NOs:46-48, respectively; (7) heavy chain CDR1-3 sequences that are substantially identical to SEQ ID NOs:51-53, respectively; and light chain CDR1-3 sequences that are substantially identical to SEQ ID NOs:54-56, respectively; (8) heavy chain CDR1-3 sequences that are substantially identical to SEQ ID NOs:59-61, respectively; and light chain CDR1-3 sequences that are substantially identical to SEQ ID NOs62-64:, respectively; (9) heavy chain CDR1-3 sequences that are substantially identical to SEQ ID NOs:67-69, respectively; and light chain CDR1-3 sequences that are substantially identical to SEQ ID NOs:70-72, respectively; (10) heavy chain CDR1-3 sequences that are substantially identical to SEQ ID NOs:75-77, respectively; and light chain CDR1-3 sequences that are substantially identical to SEQ ID NOs:78-80, respectively; (11) heavy chain CDR1-3 sequences that are substantially identical to SEQ ID NOs:83-85, respectively; and light chain CDR1-3 sequences that are substantially identical to SEQ ID NOs:86-88, respectively; (12) heavy chain CDR1-3 sequences that are substantially identical to SEQ ID NOs:91-93, respectively; and light chain CDR1-3 sequences that are substantially identical to SEQ ID NOs:94-96, respectively; (13) heavy chain CDR1-3 sequences that are substantially identical to SEQ ID NOs:99-101, respectively; and light chain CDR1-3 sequences that are substantially identical to SEQ ID NOs:102-104, respectively; (14) heavy chain CDR1-3 sequences that are substantially identical to SEQ ID NOs:107-109, respectively; and light chain CDR1-3 sequences that are substantially identical to SEQ ID NOs:110-112, respectively; (15) heavy chain CDR1-3 sequences that are substantially identical to SEQ ID NOs:115-117, respectively; and light chain CDR1-3 sequences that are substantially identical to SEQ ID NOs:118-120, respectively; (16) heavy chain CDR1-3 sequences that are substantially identical to SEQ ID NOs:123-125, respectively; and light chain CDR1-3 sequences that are substantially identical to SEQ ID NOs:126-128, respectively; (17) heavy chain CDR1-3 sequences that are substantially identical to SEQ ID NOs:131-133, respectively; and light chain CDR1-3 sequences that are substantially identical to SEQ ID NOs:134-136, respectively; or (18) heavy chain CDR1-3 sequences that are substantially identical to SEQ ID NOs:139-141, respectively; and light chain CDR1-3 sequences that are substantially identical to SEQ ID NOs:142-144, respectively.

In some embodiments, the antibody of the invention comprises the heavy chain CDR1-CDR3 and light chain CDR1-CDR3 sequences that are respectively identical to the sequences shown in (1) SEQ ID NOs:3-5 and SEQ ID NOs:6-8 (antibody P11); (2) SEQ ID NOs:11-13 and SEQ ID NOs:14-16 (antibody P17); (3) SEQ ID NOs:19-21 and SEQ ID NOs:22-24 (antibody P28); (4) SEQ ID NOs:27-29 and SEQ ID NOs:30-32 (antibody P130); (5) SEQ ID NOs:35-37 and SEQ ID NOs:38-40 (antibody P240); (6) SEQ ID NOs:43-45 and SEQ ID NOs:46-48 (antibody P1187); (7) SEQ ID NOs:51-53 and SEQ ID NOs:54-56 (antibody 1707); (8) SEQ ID NOs:59-61 and SEQ ID NOs62-64:(antibody P1955); (9) SEQ ID NOs:67-69 and SEQ ID NOs:70-72 (antibody P2388); (10) SEQ ID NOs:75-77 and SEQ ID NOs:78-80 (antibody P2653); (11) SEQ ID NOs:83-85 and SEQ ID NOs:86-88:(antibody P2861); (12) SEQ ID NOs:91-93 and SEQ ID NOs:94-96 (antibody P3208); (13) SEQ ID NOs:99-101 and SEQ ID NOs:102-104 (antibody N277); (14) SEQ ID NOs:107-109 and SEQ ID NOs:110-112 (antibody N350); (15) SEQ ID NOs:115-117 and SEQ ID NOs:118-120 (antibody N484); (16) SEQ ID NOs:123-125 and SEQ ID NOs:126-128 (antibody N2282); (17) SEQ ID NOs:131-133 and SEQ ID NOs:134-136 (antibody N2430); or (18) SEQ ID NOs:139-141 and SEQ ID NOs:142-144 (antibody E2810).

In other embodiments, the antibody of the invention that specifically binds to human PTPRG comprises a mature heavy chain variable region having an amino acid sequence substantially identical to SEQ ID NO:1 and a mature light chain variable region having an amino acid sequence substantially identical to SEQ ID NO:2; a mature heavy chain variable region having an amino acid sequence substantially identical to SEQ ID NO:9 and a mature light chain variable region having an amino acid sequence substantially identical to SEQ ID NO: 10; a mature heavy chain variable region having an amino acid sequence substantially identical to SEQ ID NO:17 and a mature light chain variable region having an amino acid sequence substantially identical to SEQ ID NO:18; a mature heavy chain variable region having an amino acid sequence substantially identical to SEQ ID NO:25 and a mature light chain variable region having an amino acid sequence substantially identical to SEQ ID NO:26; a mature heavy chain variable region having an amino acid sequence substantially identical to SEQ ID NO:33 and a mature light chain variable region having an amino acid sequence substantially identical to SEQ ID NO:34; a mature heavy chain variable region having an amino acid sequence substantially identical to SEQ ID NO:41 and a mature light chain variable region having an amino acid sequence substantially identical to SEQ ID NO:42; a mature heavy chain variable region having an amino acid sequence substantially identical to SEQ ID NO:49 and a mature light chain variable region having an amino acid sequence substantially identical to SEQ ID NO:50; a mature heavy chain variable region having an amino acid sequence substantially identical to SEQ ID NO:57 and a mature light chain variable region having an amino acid sequence substantially identical to SEQ ID NO:58; a mature heavy chain variable region having an amino acid sequence substantially identical to SEQ ID NO:65 and a mature light chain variable region having an amino acid sequence substantially identical to SEQ ID NO:66; a mature heavy chain variable region having an amino acid sequence substantially identical to SEQ ID NO:73 and a mature light chain variable region having an amino acid sequence substantially identical to SEQ ID NO:74; a mature heavy chain variable region having an amino acid sequence substantially identical to SEQ ID NO:81 and a mature light chain variable region having an amino acid sequence substantially identical to SEQ ID NO:82; or a mature heavy chain variable region having an amino acid sequence substantially identical to SEQ ID NO:89 and a mature light chain variable region having an amino acid sequence substantially identical to SEQ ID NO:90. In some embodiments, the antibody of the invention comprises a mature heavy chain variable region having an amino acid sequence at least 90% identical to SEQ ID NO:1, and a mature light chain variable region having an amino acid sequence at least 90% identical to SEQ ID NO:2; a mature heavy chain variable region having an amino acid sequence at least 90% identical to SEQ ID NO:9, and a mature light chain variable region having an amino acid sequence at least 90% identical to SEQ ID NO:10; a mature heavy chain variable region having an amino acid sequence at least 90% identical to SEQ ID NO:17, and a mature light chain variable region having an amino acid sequence at least 90% identical to SEQ ID NO:18; a mature heavy chain variable region having an amino acid sequence at least 90% identical to SEQ ID NO:25, and a mature light chain variable region having an amino acid sequence at least 90% identical to SEQ ID NO:26; a mature heavy chain variable region having an amino acid sequence at least 90% identical to SEQ ID NO:33, and a mature light chain variable region having an amino acid sequence at least 90% identical to SEQ ID NO:34; a mature heavy chain variable region having an amino acid sequence at least 90% identical to SEQ ID NO:41, and a mature light chain variable region having an amino acid sequence at least 90% identical to SEQ ID NO:42; a mature heavy chain variable region having an amino acid sequence at least 90% identical to SEQ ID NO:49, and a mature light chain variable region having an amino acid sequence at least 90% identical to SEQ ID NO:50; a mature heavy chain variable region having an amino acid sequence at least 90% identical to SEQ ID NO:57, and a mature light chain variable region having an amino acid sequence at least 90% identical to SEQ ID NO:58; a mature heavy chain variable region having an amino acid sequence at least 90% identical to SEQ ID NO:65, and a mature light chain variable region having an amino acid sequence at least 90% identical to SEQ ID NO:66; a mature heavy chain variable region having an amino acid sequence at least 90% identical to SEQ ID NO:73, and a mature light chain variable region having an amino acid sequence at least 90% identical to SEQ ID NO:74; a mature heavy chain variable region having an amino acid sequence at least 90% identical to SEQ ID NO:81, and a mature light chain variable region having an amino acid sequence at least 90% identical to SEQ ID NO:82; or a mature heavy chain variable region having an amino acid sequence at least 90% identical to SEQ ID NO:89, and a mature light chain variable region having an amino acid sequence at least 90% identical to SEQ ID NO:90. In some embodiments, the percentage identity can be at least 91%, at least 92%, at least 93%, at least 94%, at least 95%, at least 96%, at least 97%, at least 98%, or at least 99%, or 100%. In some antibodies of the invention, the mature heavy chain variable region has an amino acid sequence of SEQ ID NO:1 and the mature light chain variable region has an amino acid sequence of SEQ ID NO:2; the mature heavy chain variable region has an amino acid sequence of SEQ ID NO:9 and the mature light chain variable region has an amino acid sequence of SEQ ID NO:10; the mature heavy chain variable region has an amino acid sequence of SEQ ID NO: 17 and the mature light chain variable region has an amino acid sequence of SEQ ID NO:18; the mature heavy chain variable region has an amino acid sequence of SEQ ID NO:25 and the mature light chain variable region has an amino acid sequence of SEQ ID NO:26; the mature heavy chain variable region has an amino acid sequence of SEQ ID NO:33 and the mature light chain variable region has an amino acid sequence of SEQ ID NO:34; the mature heavy chain variable region has an amino acid sequence of SEQ ID NO:41 and the mature light chain variable region has an amino acid sequence of SEQ ID NO:42; the mature heavy chain variable region has an amino acid sequence of SEQ ID NO:49 and the mature light chain variable region has an amino acid sequence of SEQ ID NO:50; the mature heavy chain variable region has an amino acid sequence of SEQ ID NO:57 and the mature light chain variable region has an amino acid sequence of SEQ ID NO:58; the mature heavy chain variable region has an amino acid sequence of SEQ ID NO:65 and the mature light chain variable region has an amino acid sequence of SEQ ID NO:66; the mature heavy chain variable region has an amino acid sequence of SEQ ID NO:73 and the mature light chain variable region has an amino acid sequence of SEQ ID NO:74; the mature heavy chain variable region has an amino acid sequence of SEQ ID NO:81 and the mature light chain variable region has an amino acid sequence of SEQ ID NO:82; or the mature heavy chain variable region has an amino acid sequence of SEQ ID NO:89 and the mature light chain variable region has an amino acid sequence of SEQ ID NO:90.

In other embodiments, the antibody of the invention that specifically binds to human Nectin-2 comprises a mature heavy chain variable region having an amino acid sequence substantially identical to SEQ ID NO:97 and a mature light chain variable region having an amino acid sequence substantially identical to SEQ ID NO:98; a mature heavy chain variable region having an amino acid sequence substantially identical to SEQ ID NO:105 and a mature light chain variable region having an amino acid sequence substantially identical to SEQ ID NO:106; a mature heavy chain variable region having an amino acid sequence substantially identical to SEQ ID NO:113 and a mature light chain variable region having an amino acid sequence substantially identical to SEQ ID NO:114; a mature heavy chain variable region having an amino acid sequence substantially identical to SEQ ID NO:121 and a mature light chain variable region having an amino acid sequence substantially identical to SEQ ID NO:122; or a mature heavy chain variable region having an amino acid sequence substantially identical to SEQ ID NO:129 and a mature light chain variable region having an amino acid sequence substantially identical to SEQ ID NO:130. In some embodiments, the antibody of the invention comprises a mature heavy chain variable region having an amino acid sequence at least 90% identical to SEQ ID NO:97, and a mature light chain variable region having an amino acid sequence at least 90% identical to SEQ ID NO:98; a mature heavy chain variable region having an amino acid sequence at least 90% identical to SEQ ID NO:105, and a mature light chain variable region having an amino acid sequence at least 90% identical to SEQ ID NO:106; a mature heavy chain variable region having an amino acid sequence at least 90% identical to SEQ ID NO:113, and a mature light chain variable region having an amino acid sequence at least 90% identical to SEQ ID NO:114; a mature heavy chain variable region having an amino acid sequence at least 90% identical to SEQ ID NO:121, and a mature light chain variable region having an amino acid sequence at least 90% identical to SEQ ID NO:122; or a mature heavy chain variable region having an amino acid sequence at least 90% identical to SEQ ID NO:129, and a mature light chain variable region having an amino acid sequence at least 90% identical to SEQ ID NO:130. In some embodiments, the percentage identity can be at least 91%, at least 92%, at least 93%, at least 94%, at least 95%, at least 96%, at least 97%, at least 98%, or at least 99%, or 100%. In some antibodies of the invention, the mature heavy chain variable region has an amino acid sequence of SEQ ID NO:97 and the mature light chain variable region has an amino acid sequence of SEQ ID NO:98; the mature heavy chain variable region has an amino acid sequence of SEQ ID NO:105 and the mature light chain variable region has an amino acid sequence of SEQ ID NO:106; the mature heavy chain variable region has an amino acid sequence of SEQ ID NO:113 and the mature light chain variable region has an amino acid sequence of SEQ ID NO:114; the mature heavy chain variable region has an amino acid sequence of SEQ ID NO:121 and the mature light chain variable region has an amino acid sequence of SEQ ID NO:122; or the mature heavy chain variable region has an amino acid sequence of SEQ ID NO:129 and the mature light chain variable region has an amino acid sequence of SEQ ID NO:130.

In other embodiments, the antibody of the invention that specifically binds to human endoglin comprises a mature heavy chain variable region having an amino acid sequence substantially identical to SEQ ID NO:137 and a mature light chain variable region having an amino acid sequence substantially identical to SEQ ID NO:138. In some embodiments, the antibody of the invention comprises a mature heavy chain variable region having an amino acid sequence at least 90% identical to SEQ ID NO:137 and a mature light chain variable region having an amino acid sequence at least 90% identical to SEQ ID NO:138. In some embodiments, the percentage identity can be at least 91%, at least 92%, at least 93%, at least 94%, at least 95%, at least 96%, at least 97%, at least 98%, or at least 99%, or even 100%. In some antibodies of the invention, the mature heavy chain variable region has an amino acid sequence of SEQ ID NO:137 and the mature light chain variable region has an amino acid sequence of SEQ ID NO:138.

In the various embodiments, percent (%) identity of peptide sequences can be calculated, for example, as 100×[(identical positions)/min(TGA, TGB)], where TGA and TGB are the sum of the number of residues and internal gap positions in peptide sequences A and B in the alignment that minimizes TGA and TGB. See, e.g., Russell et al, J. Mol. Biol., 244: 332-350 (1994).

The antibody, antibody fragment, antibody-based binding protein, ADC or CAR of the invention of the invention can be any antibody including a full length antibody or an antibody fragment that specifically recognizes or binds to an acute myeloid leukemia cell surface antigen, for example PTPRG, Nectin-1, or endoglin. For example, the antibody, antibody fragment or antibody-based binding protein can be polyclonal, monoclonal, recombinant, chimeric, or humanized. Furthermore, the antibody can be of any isotype including without limitation IgA, IgD, IgE, IgG, or IgM. Thus, for example, the antibody can be any IgA such as IgAQ1 or IgA2, or any IgG such as IgG1, IgG2, IgG3, IgG4, or synthetic IgG. The antibody can also be any antibody fragment or antibody-based binding protein having specificity for the extracellular domain of human ROR2, such as F(ab)2, Fv, scFv, IgGACH2, F(ab′)2, scFv2CH3, Fab, VL, VH, scFv4, scFv3, scFv2, dsFv, Fv, scFv-Fc, (scFv)2, a diabody, and a bivalent antibody. The antibody can be any modified or synthetic antibody, including, but not limited to, non-depleting IgG antibodies, CARs, or other Fc or Fab variants of antibodies.

In addition to a heavy chain as described above, the antibody, antibody-based binding proteins or antibody fragments thereof of the invention can further comprise a light chain selected from a Fab library using sequential naive chain shuffling. Likewise, in addition to a light chain as described above, the antibody of the invention can further comprise a heavy chain selected from a Fab library using sequential naive chain shuffling.

The antibody, antibody-based binding protein or antibody fragment thereof of the invention can be produced by any suitable technique, for example, using any suitable eukaryotic or non-eukaryotic expression system. In certain embodiments, the antibody, antibody-based binding protein or antibody fragment thereof is produced using a mammalian expression system. Some specific techniques for generating the antibodies or antigen-binding fragments of the invention are exemplified herein. In some embodiments, the antibodies or antigen-binding fragments of the invention can be produced using a suitable non-eukaryotic expression system such as a bacterial expression system. Bacterial expression systems can be used to produce fragments such as a F(ab)2, Fv, scFv, IgGACH2, F(ab′)2, scFv2CH3, Fab, VL, VH, scFv4, scFv3, scFv2, dsFv, Fv, scFv-Fc, (scFv)2, and diabodies. Techniques for altering DNA coding sequences to produce such fragments are known in the art.

The antibodies, antibody-based binding proteins or antibody fragments thereof of the invention can be conjugated to a synthetic molecule using any type of suitable conjugation. Recombinant engineering and incorporated selenocysteine (e.g., as described in U.S. Pat. No. 8,916,159 issued on Dec. 23, 2014) can be used to conjugate a synthetic molecule. Other methods of conjugation can include covalent coupling to native or engineered lysine side-chain amines or cysteine side-chain thiols. See, e.g., Wu et al., Nat. Biotechnol. 23: 1 137-1 146 (2005).

In an embodiment, the antibodies, antibody-based binding proteins or antibody fragments thereof of the invention conjugated to a synthetic molecule (called “ADC” for antibody drug conjugate with the synthetic molecule being a toxin) are obtained by means of site-specific chemical or enzymatic antibody conjugation.

The synthetic molecule can be any molecule such as one targeting a tumor. In some embodiments, the synthetic molecule for conjugation to the antibody is a protein (e.g., an antibody) or an RNA or DNA aptamer. In one embodiment, the antibodies, antibody-based binding proteins or antibody fragments thereof of the invention conjugated to a synthetic molecule have the general formula A-(L-P)ₙ, in which: A is an antibody, antibody-based binding protein or antibody fragment thereof as described herein, L is one or more linkers, P is one or more payloads selected from the group consisting of a label and a cytotoxic or cytostatic agent, and in which n is an integer between ≥1 and ≤10. In this embodiment, the linker may comprise, or consists of, at least one selected from the group consisting of: an oligopeptide linker (including cleavable and non-cleavable oligopeptide linkers), a hydrazine linker, a thiourea linker, a self-immolative linker, a succinimidyl trans-4-(maleimidylmethyl)cyclohexane-1-carboxylate (SMCC) linker, a maleimide linker, a disulfide linker, a thioether linker, and/or a maleimide linker.

The skilled person understands that further linkers may be suitable. Such linkers may be non-cleavable or may be cleaved by changes in pH, redox potential or specific intracellular enzymes. Cleavable oligopeptide linkers include protease- or matrix metalloprotease-cleavable linkers. It is understood that the linker may comprise combinations of the above. For example, the linker may be a valine-citrulline PAB linker.

In various embodiments, suitable synthetic molecules (“payloads”) for conjugation to the antibody include, e.g., therapeutic agents such as cytotoxic, cytostatic, or antiangiogenic agents, radioisotopes, and liposomes. A cytotoxic agent can be a plant, fungal, or bacterial molecule. In some embodiments, the cytotoxic agent for conjugation to the antibody of the invention is a small molecular weight toxin (MW<2′000 Dalton, preferably MW<1′000 Dalton), a peptide toxin, or a protein toxin. Many specific examples of these toxins are well known in the art. See, e.g., Dyba et al., Curr. Pharm. Des. 10:2311-34, 2004; Kuyucak et al., Future Med. Chem. 6:1645-58, 2014; Beraud et al., Inflamm. Allergy Drug Targets. 10:322-42, 2011; and Middlebrook et al., Microbiol. Rev. 48:199-221, 1984. In some embodiments, a therapeutic agent is conjugated to the antibody. For example, the therapeutic agent can be a maytansinoid (e.g., maytansinol or DM1 maytansinoid), a taxane, a calicheamicin, a cemadotin, a monomethylauristatin (e.g., monomethylauristatin E or monomethylauristatin F), a pyrrolobenzodiazepine (PBD), an anthracycline, such as a derivative of the highly potent anthracycline PNU-159682. Therapeutic agents also include vincristine and prednisone. In various embodiments, the therapeutic agent that may be employed in the invention can be an antimetabolite (e.g., an antifolate such as methotrexate, a fluoropyrimidine such as 5-fluorouracil, cytosine arabinoside, or an analogue of purine or adenosine); an intercalating agent an intercalating agent (for example, an anthracycline such as doxorubicin, nemorubicine, or preferably a derivative of PNU-159682), daunomycin, epirabicin, idarubicin, mitomycin-C, dactinomycin, or mithramycin, or other intercalating agents such as pyrrolobenzodiazepine); a DNA-reactive agent such as calicheamicins, tiancimycins, and other enediynes; a platinum derivative (e.g., cisplatin or carboplatin); an alkylating agent (e.g., nitrogen mustard, melphalan, chlorambucil, busulphan, cyclophosphamide, ifosfamide nitrosoureas or thiotepa); an RNA polymerase inhibitor such as α-amanitin; an antimitotic agent (e.g., a vinca alkaloid such as vincristine, or a taxoid such as paclitaxel or docetaxel); a topoisomerase inhibitor (for example, etoposide, teniposide, amsacrine, topotecan); a cell cycle inhibitor (for example, a flavopyridol); or a microbtubule agent (e.g., an epothilone, a tubulysine, a pre-tubulysine, a discodermolide analog, or an eleutherobin analog). A therapeutic agent can be a proteosome inhibitor or a topoisomerase inhibitor such as bortezomib, amsacrine, etoposide, etoposide phosphate, teniposide, or doxorubicin. Therapeutic radioisotopes include iodine (¹³¹I), yttrium (⁹⁰Y), lutetium (¹⁷⁷Lu), actinium (²²⁵Ac), praseodymium, astatine (At), rhenium (Re), bismuth (Bi or Bi), and rhodium (Rh). Antiangiogenic agents include linomide, bevacuzimab, angiostatin, and razoxane.

In some embodiments, the synthetic molecule can be conjugated to any antibody, antibody-based binding protein, or antibody-fragment. In some embodiments, the synthetic molecule can be a label. Labels can be useful in diagnostic applications and can include, for example, contrast agents. A contrast agent can be a radioisotope label such as iodine (¹³¹I or ¹²⁵I), indium (¹¹¹In), technetium (⁹⁹Tc), phosphorus (³²P), carbon (¹⁴C), tritium (3H), other radioisotope (e.g., a radioactive ion), or a therapeutic radioisotope such as one of the therapeutic radioisotopes listed above. Additionally, contrast agents can include radiopaque materials, magnetic resonance imaging (MRI) agents, ultrasound imaging agents, and any other contrast agents suitable for detection by a device that images an animal body. A synthetic molecule can also be a fluorescent label, a biologically active enzyme label, a luminescent label, or a chromophore label.

In some other embodiments, the synthetic molecule can be a liposome, as described in Bendas, BioDrugs, 15: 215-224, 2001. In such embodiments, the antibody can be conjugated to a colloidal particle, e.g., a liposome, and used for controlled delivery of an agent to diseased cells. In preparing an antibody conjugated to a liposome, e.g., an immunoliposome, an agent such as a chemotherapeutic or other drug can be entrapped in the liposome for delivery to a target cell. In some embodiments, the antibodies, antibody-based binding proteins or antibody fragments thereof of the invention can also have specificity for one or more antigens in addition to PTPRG, Nectin-1, or endoglin. For example, the antibody of the invention can be engineered (e.g., as a bivalent diabody or a conjugated Fab dimer or trimer) to have specificity for PTPRG, Nectin-1, or endoglin and another tumor antigen, e.g., an antigen associated with a hematologic malignancy, for example acute myeloid leukemia (AML). The antibody can be engineered to have specificity for PTPRG, Nectin-1, or endoglin and an antigen that promotes activation or targeting of cytotoxic effector cells.

### V. Polynucleotides, Vectors and Host Cells for Producing Anti-PTPRG, Anti-Nectin-2, or Anti-Endoglin Antibodies

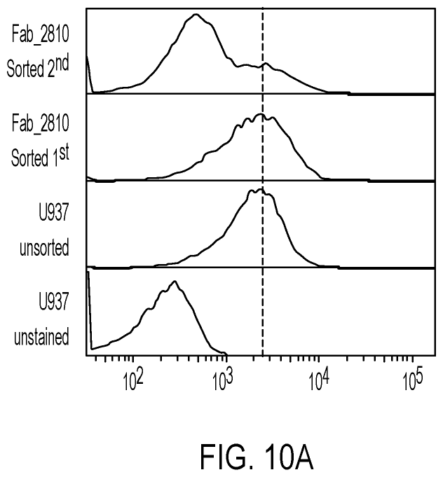

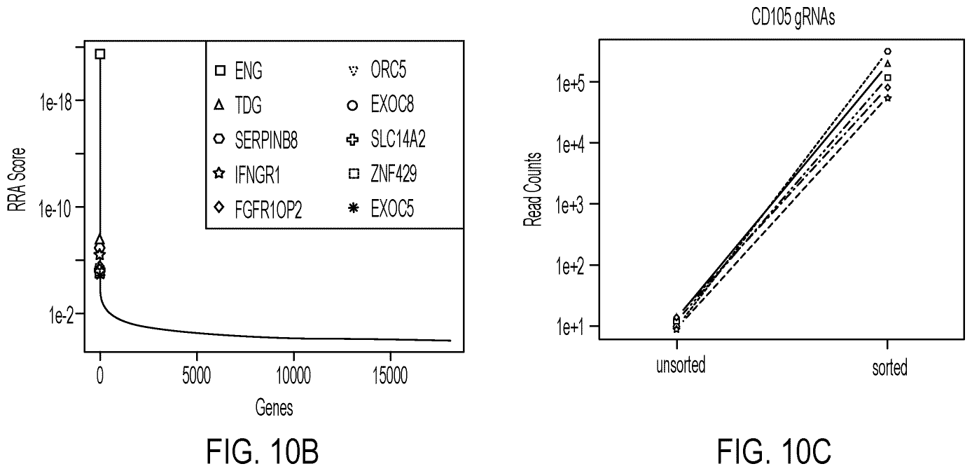

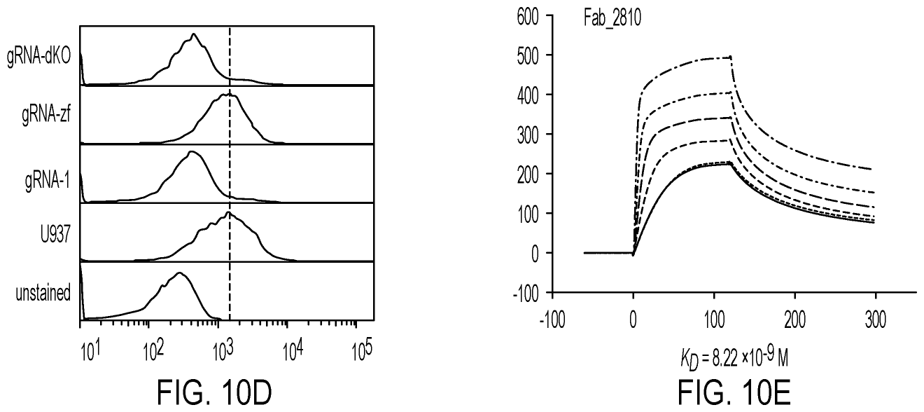

The invention provides substantially purified polynucleotides (DNA or RNA) that are identical or complementary to sequences encoding polypeptides comprising segments or domains of the antibody, antibody-based binding protein or antibody fragment thereof chains described herein. In some embodiments, the polynucleotides of the invention encode the heavy chain or light chain domains sequences shown in FIG. 10. When expressed from appropriate expression vectors, polypeptides encoded by these polynucleotides are capable of exhibiting PTPRG, Nectin-1, or endoglin antigen binding capacity.

Also provided in the invention are polynucleotides which encode at least one CDR region and usually all three CDR regions from the heavy or light chain of the antibodies described herein. Some other polynucleotides encode all or substantially all of the variable region sequence of the heavy chain and/or the light chain of the exemplified antibodies. For example, some of these polynucleotides encode the amino acid sequence of the heavy chain variable region shown in any one SEQ ID NOs: 1, 9, 17, 25, 33, 41, 49, 57, 65, 73, 81, 89, 97, 105, 113, 121, 129, and 137, and/or the amino acid sequence of the light chain variable region shown in any one SEQ ID NOs: 2, 10, 18, 26, 34, 42, 50, 58, 66, 74, 82, 90, 98, 106, 114, 122, 130, and 138. Because of the degeneracy of the code, a variety of nucleic acid sequences will encode each of the immunoglobulin amino acid sequences.

The polynucleotides of the invention can encode only the variable region sequences of the exemplified antibodies. They can also encode both a variable region and a constant region of the antibody. Some of polynucleotide sequences of the invention nucleic acids encode a mature heavy chain variable region sequence that is substantially identical (e.g., at least 80%, 90%, 95% or 99%) to the mature heavy chain variable region sequence shown in any one SEQ ID NOs: 1, 9, 17, 25, 33, 41, 49, 57, 65, 73, 81, 89, 97, 105, 113, 121, 129, and 137. Some other polynucleotide sequences encode a mature light chain variable region sequence that is substantially identical to the mature light chain variable region sequence shown in any one SEQ ID NOs: 2, 10, 18, 26, 34, 42, 50, 58, 66, 74, 82, 90, 98, 106, 114, 122, 130, and 138. Some of the polynucleotide sequences encode a polypeptide that comprises variable regions of both the heavy chain and the light chain of one of the exemplified antibodies. Some other polynucleotides encode two polypeptide segments that respectively are substantially identical to the variable regions of the heavy chain or the light chain of one of the exemplified antibodies.

The polynucleotide sequences can be produced by de novo solid-phase DNA synthesis or by PCR mutagenesis of an existing sequence (e.g., sequences as described in the Examples below) encoding an exemplified functional antibody. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, CA, 1990; Mattila et al., Nucleic Acids Res. 19:967, 1991; and Eckert et al., PCR Methods and Applications 1:17, 1991.

Also provided in the invention are expression vectors and host cells for producing the functional antibodies described herein. Specific examples of plasmid and transposon based vectors for expressing the antibodies are described in the Examples below. Various other expression vectors can also be employed to express the polynucleotides encoding the functional antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the antibody polynucleotides and polypeptides in mammalian (e.g., human) cells include pCEP4, pREP4, pThioHis A, B & C, pcDNA3.1/His, pEBVHis A, B & C (Invitrogen, San Diego, CA), MPSV vectors, and numerous other vectors known in the art for expressing other proteins. Other useful nonviral vectors comprise expression cassettes that can be mobilized with Sleeping Beauty, PiggyBack and other transposon systems. Useful viral vectors include vectors based on lentiviruses or other retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a functional antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of a functional antibody chain or fragment. These elements typically include an ATG initiation codon and adjacent ribosome binding site (Kozak consensus sequence) or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted functional antibody sequences. More often, the inserted functional antibody sequences are linked to a signal sequences before inclusion in the vector. Vectors to be used to receive sequences encoding the functional antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human, and preferably of human IgG1 antibodies.

The host cells for harboring and expressing the functional antibody chains can be either prokaryotic or eukaryotic. In some preferred embodiments, mammalian host cells are used to express and to produce the antibody polypeptides of the present invention. For example, they can be either a hybridoma cell line expressing endogenous immunoglobulin genes or a mammalian cell line harboring an exogenous expression vector. These include any normal mortal or normal or abnormal immortal animal or human cell. In addition to the cell lines exemplified herein, a number of other suitable host cell lines capable of secreting intact immunoglobulins are also known in the art. These include, e.g., the CHO cell lines, various HEK 293 cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer, and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, EF1α and human UbC promoters exemplified herein, the metallothionein promoter, the constitutive adenovirus major late promoter, the dexamethasone-inducible MMTV promoter, the SV40 promoter, the MRP pol III promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transformation is commonly utilized for prokaryotic cells, whereas calcium phosphate treatment or electroporation may be used for other cellular hosts (see generally Brent et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate for the cell type.

The invention further provides eukaryotic or non-eukaryotic cells (e.g., T lymphocytes) that have been recombinantly engineered to produce the antibodies, antibody-based binding proteins or antibody fragments thereof of the invention. The eukaryotic or non-eukaryotic cells can be used as an expression system to produce the antibody of the invention. In some embodiments, the invention provides PTPRG, Nectin-1, or endoglin targeted immune cells that are engineered to recombinantly express an PTPRG, Nectin-1, or endoglin specific antibody of the invention. For example, the invention provides a T cell or NK cell engineered to express an antibody of the invention (e.g., an scFv, scFv-Fc, or (scFv)2), which is linked to a synthetic molecule containing one or more of the following domains: a spacer or hinge region (e.g., a CD28 sequence or a IgG4 hinge-Fc sequence), a transmembrane region (e.g., a transmembrane canonical domain), and an intracellular T-cell receptor (TCR) signaling domain, an intracellular T-cell costimulatory receptor signaling domain, or a signaling domain of a protein downstream of TCR or T-cell costimulatory receptor, thereby forming a chimeric antigen receptor (CAR) or T-body. Intracellular TCR signaling domains that can be included in a CAR (or T-body) include, but are not limited to, CD3ζ, FcR-γ, and Syk-PT signaling domains as well as the CD28, 4-1BB, and CD134 co-signaling domains. Methods for constructing T cells and NK cells expressing a CAR are known in the art. See, e.g., Marcu-Malina et al., Expert Opinion on Biological Therapy, Vol. 9, No. 5 (posted online on Apr. 16, 2009) and Lu H, Zhao X, Li Z, Hu Y, Wang H. Front Oncol. 2021 Aug. 6; 11:720501.

### VI. Therapeutic and Diagnostic Applications

In one aspect, the invention provides methods for inhibiting cells that express PTPRG, Nectin-1, or endoglin (PTPRG, Nectin-1, or endoglin cells, respectively) by contacting the cells with an antibody, antibody-based binding protein or antibody fragment thereof of the invention, or an antibody drug conjugate (ADC) or an engineered cell harboring a chimeric antigen receptor (CAR) described herein. The antibody, antibody-based binding protein or antibody fragment thereof can be a naked (unconjugated) molecule or an antibody molecule conjugated to a synthetic molecule, e.g., a cytotoxic, cytostatic, or antiangiogenic agent, a radioisotope, or even to a liposome. The method can be used to inhibit PTPRG, Nectin-1, or endoglin cells in vitro or in a subject (i.e., in vivo). The contacted PTPRG, Nectin-1, or endoglin cells can be in, for example, a cell culture or animal model of a disorder associated with elevated levels of PTPRG, Nectin-1, or endoglin. The methods are useful, for example, to measure and/or rank (relative to another antibody) the antibody's inhibitory activity for a specific PTPRG, Nectin-1, or endoglin cell type. Inhibiting PTPRG, Nectin-1, or endoglin cells can include blocking or reducing the activity or growth of PTPRG, Nectin-1, or endoglin cells. Inhibiting can also include the killing of PTPRG, Nectin-1, or endoglin cells. Inhibitory activity can also be mediated by recruitment of immune system effectors that attack PTPRG, Nectin-1, or endoglin, e.g., by activating constituents of the antibody-dependent cell-mediated cytotoxicity (ADCC) or complement systems.

In some related embodiments, the invention provides methods for treating a subject that has, is suspected to have, or is at risk of developing a disorder associated with elevated levels of PTPRG, Nectin-1, or endoglin. Generally, the methods include administering a pharmaceutical composition that contains a therapeutically effective amount of an isolated antibody, antibody-based binding protein, antibody fragment thereof, ADC or CAR of the invention to the subject. The antibody can be any anti-PTPRG, Nectin-1, or endoglin antibody of the invention as described herein. Thus, the antibody can be chimeric, humanized, synthetic, F(ab)2, Fv, scFv, IgGACH2, F(ab′)2, scFv2CH3, Fab, VL, VH, scFv4, scFv3, scFv2, dsFv, Fv, or (scFv)2. In some embodiments, the method includes administering an IgG, an scFv, a dsFv, a F(ab′)2, a diabody, or a bivalent antibody. The administered antibody or antigen-binding fragment can be conjugated to a synthetic molecule described above, e.g., a cytotoxic, cytostatic, or antiangiogenic agent, a therapeutic radioisotope, or a liposome. An exemplary cytotoxic agent is Pseudomonas exotoxin A (PE38). Disorders that can be treated include a hematologic malignancy, for example AML and other disorders with elevated PTPRG, Nectin-1, or endoglin expression.

In some embodiments, the invention provides methods for treating a subject that has, is suspected to have, or is at risk of developing a disorder associated with expression of PTPRG, Nectin-1, or endoglin by adoptive transfer of the genetically engineered T-cells described herein, which express an antibody or antigen-binding fragment of the invention as a chimeric antigen receptor (CAR) that selectively binds PTPRG, Nectin-1, or endoglin. Recombinant technology can be used to introduce CAR-encoding genetic material into any suitable T cells, e.g., autologous T cells from the subject to be treated, off-the-shelf allogeneic T cells, or autologous or off-the-shelf allogeneic NK cells. The T or NK cells carrying the genetic material can be expanded (e.g., in the presence of cytokines). The genetically engineered T or NK cells are transferred, typically by infusion, to the patient. The transferred T or NK cells of the invention can then mount an immune response against PTPRG, Nectin-1, or endoglin expressing cells in the subject. The adoptive transfer method can be used, for example, to treat subjects that have or are suspected to have a hematologic malignancy, for example AML.

In some embodiments, the foregoing methods of treatment can further include co-administering a second therapeutic agent for treating the disorder associated with elevated PTPRG, Nectin-1, or endoglin. For example, when the disorder to be treated involves an PTPRG, Nectin-1, or endoglin-expressing hematologic malignancy, the method can further include co-administration of a cytotoxic, cytostatic, or antiangiogenic or immune-stimulatory agent (e.g. immune-checkpoint inhibitor antibodies, for instance, but not limited to, those binding to PD1, PDL1, CTLA4, OX40, TIM3, GITR, LAG3 and the like) suitable for treating the hematologic malignancy. If the hematologic malignancy is AML, the method can further include, for example, co-administration of chemotherapy, venetoclax, or gemtuzumab ozogamicin.

In some other embodiments, the invention provides method for detecting in a biological sample an altered level of PTPRG, Nectin-1, or endoglin (e.g., cell surface PTPRG, Nectin-1, or endoglin), for example, relative to a control, either by FACS, immunohistochemistry (IHC) or Western Blotting. Generally, the method includes contacting a biological sample with an antibody, antibody-based binding protein, antibody fragment thereof of the invention and determining the amount of antibody that selectively binds to material (e.g., cells) in the sample to thereby determine the level of PTPRG, Nectin-1, or endoglin in the biological sample. A biological sample can be from a cell culture or from a test subject, e.g., a plasma or a tissue sample from a subject that has, is suspected to have, or is at risk of developing a disease or condition associated with elevated PTPRG, Nectin-1, or endoglin in a subject. A control level desirably corresponds to the PTPRG, Nectin-1, or endoglin level detected using the same antibody in a corresponding sample(s) from one or more control cultures or disease-free subjects. Methods of using the antibody of the invention to determine PTPRG, Nectin-1, or endoglin levels can include any immunoassay such as immuno- (Western) blotting, enzyme-linked immunosorbent assay (ELISA), Immunohistochemistry (IHC) and flow cytometry, e.g., fluorescence-activated cell sorting (FACS) analysis.

The methods of detection can be used to screen for the presence of a disorder associated with elevated PTPRG, Nectin-1, or endoglin. The methods include obtaining a sample from a test subject in need of screening, e.g., a subject that has, is suspected to have, or is at risk of developing a disorder associated with elevated PTPRG, Nectin-1, or endoglin. The level of PTPRG, Nectin-1, or endoglin (e.g., the amount or concentration) in the sample is measured using an antibody, antibody-based binding protein, antibody fragment thereof of the invention, and the level in the sample is compared to a control level of PTPRG, Nectin-1, or endoglin. The control level represents, for example, the mean level (e.g., the amount or concentration) in sample(s) from one or, preferably, multiple control group subjects that do not have a disorder associated with elevated PTPRG, Nectin-1, or endoglin. Alternatively, the control level can correspond to the level or mean level of PTPRG, Nectin-1, or endoglin in one or more samples taken from the test subject at one or more prior times, such as when the test subject did not have or did not exhibit, a condition associated with elevated PTPRG, Nectin-1, or endoglin. A significantly higher level of PTPRG, Nectin-1, or endoglin in the biological sample relative to the control level is indicative of a disorder associated with elevated PTPRG, Nectin-1, or endoglin in the subject.

Additionally, the methods of detection can be used to monitor the progress of a disorder associated with elevated PTPRG, Nectin-1, or endoglin. The method includes obtaining a sample from a subject in need of screening, e.g., a subject having been diagnosed or suspected to have a disorder associated with elevated PTPRG, Nectin-1, or endoglin. The level of PTPRG, Nectin-1, or endoglin in the sample is measured using an antibody, antibody-based binding protein, antibody fragment thereof of the invention, and the level in the sample is compared to a control level corresponding to the level or mean level of PTPRG, Nectin-1, or endoglin in one or more samples taken from the test subject at one or more prior times. Levels of PTPRG, Nectin-1, or endoglin that are significantly elevated or decreased relative to control indicate that the subject's disorder is deteriorating or improving, respectively. The foregoing methods of detection can be used to screen for the presence or to monitor the progress of disorders including, for example, a hematologic malignancy, for example AML.

In some embodiments, the invention provides methods for screening a subject for an altered level of PTPRG, Nectin-1, or endoglin. Generally, the methods entail administering to the subject an antibody, antibody-based binding protein, antibody fragment thereof of the invention that is conjugated to a label (e.g., a contrast agent), imaging the subject in a manner suitable for detecting the label, and determining whether a region in the subject has an altered density or concentration of label as compared to the background level of label in proximal tissue. Alternatively, the methods include determining whether there is an altered density or concentration of label in a region as compared to the density or concentration of label previously detected in the same region of the subject. Methods of imaging a subject can include x-ray imaging, x-ray computed tomography (CT) imaging (e.g., CT angiography (CTA) imaging), magnetic resonance (MR) imaging, magnetic resonance angiography (MRA), nuclear medicine, ultrasound (US) imaging, optical imaging, elastography, infrared imaging, microwave imaging, and the like, as appropriate for detecting the label conjugated to the antibody. In a preferred embodiment, the subject has, is suspected to have, or is at risk of developing an PTPRG, Nectin-1, or endoglin-expressing tumor, such as a hematologic malignancy, for example AML, and the method is used to screen for or detect the presence of the tumor. In another embodiment, the method can be used to monitor the size or density of a PTPRG, Nectin-1, or endoglin-expressing tumor over time, e.g., during a course of treatment.

### VII. Pharmaceutical Compositions and Combinations

In another aspect, the invention provides pharmaceutical compositions that contain an antibody, an antibody fragment, an antibody-based binding protein, or an ADC as described herein and a pharmaceutically acceptable carrier. Pharmaceutical compositions can be prepared from any of the antibodies or related compounds described herein. Exemplary compositions include one or more of an antibody comprising a mature heavy chain variable region having an amino acid sequence of SEQ ID NO:1 and a mature light chain variable region having an amino acid sequence of SEQ ID NO:2; a mature heavy chain variable region having an amino acid sequence of SEQ ID NO:9 and a mature light chain variable region having an amino acid sequence of SEQ ID NO:10; a mature heavy chain variable region having an amino acid sequence of SEQ ID NO: 17 and a mature light chain variable region having an amino acid sequence of SEQ ID NO:18; a mature heavy chain variable region having an amino acid sequence of SEQ ID NO:25 and a mature light chain variable region having an amino acid sequence of SEQ ID NO:26; a mature heavy chain variable region having an amino acid sequence of SEQ ID NO:33 and a mature light chain variable region having an amino acid sequence of SEQ ID NO:34; a mature heavy chain variable region having an amino acid sequence of SEQ ID NO:41 and a mature light chain variable region having an amino acid sequence of SEQ ID NO:42; a mature heavy chain variable region having an amino acid sequence of SEQ ID NO:49 and a mature light chain variable region having an amino acid sequence of SEQ ID NO:50; a mature heavy chain variable region having an amino acid sequence of SEQ ID NO: 57 and a mature light chain variable region having an amino acid sequence of SEQ ID NO:58; a mature heavy chain variable region having an amino acid sequence of SEQ ID NO:65 and a mature light chain variable region having an amino acid sequence of SEQ ID NO:66; a mature heavy chain variable region having an amino acid sequence of SEQ ID NO:73 and a mature light chain variable region having an amino acid sequence of SEQ ID NO:74; a mature heavy chain variable region having an amino acid sequence of SEQ ID NO:81 and a mature light chain variable region having an amino acid sequence of SEQ ID NO:82; or a mature heavy chain variable region having an amino acid sequence of SEQ ID NO: 89 and a mature light chain variable region having an amino acid sequence of SEQ ID NO:90; a mature heavy chain variable region having an amino acid sequence of SEQ ID NO:97 and the mature light chain variable region has an amino acid sequence of SEQ ID NO:98; a mature heavy chain variable region having an amino acid sequence of SEQ ID NO:105 and a mature light chain variable region having an amino acid sequence of SEQ ID NO:106; a mature heavy chain variable region having an amino acid sequence of SEQ ID NO:113 and a mature light chain variable region having an amino acid sequence of SEQ ID NO:114; a mature heavy chain variable region having an amino acid sequence of SEQ ID NO:121 and a mature light chain variable region having an amino acid sequence of SEQ ID NO:122; or a mature heavy chain variable region having an amino acid sequence of SEQ ID NO:129 and a mature light chain variable region having an amino acid sequence of SEQ ID NO:130; and a mature heavy chain variable region has an amino acid sequence of SEQ ID NO:137 and a mature light chain variable region having an amino acid sequence of SEQ ID NO:138.

Other exemplary compositions of the invention can contain an antibody having one, two, three, four, five, or six CDRs selected from the group consisting of SEQ ID NOs:3-5 and SEQ ID NOs:6-8. Other exemplary compositions of the invention can contain an antibody having one, two, three, four, five, or six CDRs selected from the group consisting of SEQ ID NOs:11-13 and SEQ ID NOs:14-16. Other exemplary compositions of the invention can contain an antibody having one, two, three, four, five, or six CDRs selected from the group consisting of SEQ ID NOs:19-21 and SEQ ID NOs:22-24. Other exemplary compositions of the invention can contain an antibody having one, two, three, four, five, or six CDRs selected from the group consisting of SEQ ID NOs:27-29 and SEQ ID NOs:30-32. Other exemplary compositions of the invention can contain an antibody having one, two, three, four, five, or six CDRs selected from the group consisting of SEQ ID NOs:35-37 and SEQ ID NOs:38-40. Other exemplary compositions of the invention can contain an antibody having one, two, three, four, five, or six CDRs selected from the group consisting of SEQ ID NOs:43-45 and SEQ ID NOs:46-48. Other exemplary compositions of the invention can contain an antibody having one, two, three, four, five, or six CDRs selected from the group consisting of SEQ ID NOs:51-53 and SEQ ID NOs:54-56. Other exemplary compositions of the invention can contain an antibody having one, two, three, four, five, or six CDRs selected from the group consisting of SEQ ID NOs:59-61 and SEQ ID NOs:62-64. Other exemplary compositions of the invention can contain an antibody having one, two, three, four, five, or six CDRs selected from the group consisting of SEQ ID NOs:67-69 and SEQ ID NOs:70-72. Other exemplary compositions of the invention can contain an antibody having one, two, three, four, five, or six CDRs selected from the group consisting of SEQ ID NOs:75-77 and SEQ ID NOs:78-80. Other exemplary compositions of the invention can contain an antibody having one, two, three, four, five, or six CDRs selected from the group consisting of SEQ ID NOs:83-85 and SEQ ID NOs:86-88. Other exemplary compositions of the invention can contain an antibody having one, two, three, four, five, or six CDRs selected from the group consisting of SEQ ID NOs:91-93 and SEQ ID NOs:94-96. Other exemplary compositions of the invention can contain an antibody having one, two, three, four, five, or six CDRs selected from the group consisting of SEQ ID NOs:107-109 and SEQ ID NOs:110-112. Other exemplary compositions of the invention can contain an antibody having one, two, three, four, five, or six CDRs selected from the group consisting of SEQ ID NOs:115-117 and SEQ ID NOs:118-120. Other exemplary compositions of the invention can contain an antibody having one, two, three, four, five, or six CDRs selected from the group consisting of SEQ ID NOs:123-125 and SEQ ID NOs:126-128. Other exemplary compositions of the invention can contain an antibody having one, two, three, four, five, or six CDRs selected from the group consisting of SEQ ID NOs:131-133 and SEQ ID NOs:134-136. Other exemplary compositions of the invention can contain an antibody having one, two, three, four, five, or six CDRs selected from the group consisting of SEQ ID NOs:139-141 and SEQ ID NOs:142-144. In some embodiments, however, the antibody includes three CDR sequences of the exemplified light or heavy chains shown in FIG. 13. These include the heavy chain CDR1, CDR2 and CDR3 sequences and light chain CDR1, CDR2 and CDR3 sequences respectively shown in (1) SEQ ID NOs:3-5 and SEQ ID NOs:6-8 (antibody P11); (2) SEQ ID NOs:11-13 and SEQ ID NOs:14-16 (antibody P17); (3) SEQ ID NOs:19-21 and SEQ ID NOs:22-24 (antibody P28); (4) SEQ ID NOs:27-29 and SEQ ID NOs:30-32 (antibody P130); (5) SEQ ID NOs:35-37 and SEQ ID NOs:38-40 (antibody P240); (6) SEQ ID NOs:43-45 and SEQ ID NOs:46-48 (antibody P1187); (7) SEQ ID NOs:51-53 and SEQ ID NOs:54-56 (antibody 1707); (8) SEQ ID NOs:59-61 and SEQ ID NOs62-64:(antibody P1955); (9) SEQ ID NOs:67-69 and SEQ ID NOs:70-72 (antibody P2388); (10) SEQ ID NOs:75-77 and SEQ ID NOs:78-80 (antibody P2653); (11) SEQ ID NOs:83-85 and SEQ ID NOs:86-88:(antibody P2861); (12) SEQ ID NOs:91-93 and SEQ ID NOs:94-96 (antibody P3208); (13) SEQ ID NOs:99-101 and SEQ ID NOs:102-104 (antibody N277); (14) SEQ ID NOs:107-109 and SEQ ID NOs: 110-112 (antibody N350); (15) SEQ ID NOs:115-117 and SEQ ID NOs:118-120 (antibody N484); (16) SEQ ID NOs:123-125 and SEQ ID NOs:126-128 (antibody N2282); (17) SEQ ID NOs:131-133 and SEQ ID NOs:134-136 (antibody N2430); or (18) SEQ ID NOs:139-141 and SEQ ID NOs:142-144 (antibody E2810). In some embodiments, the pharmaceutical composition includes an antibody having six CDR sequences of the same antibody exemplified in FIG. 13, e.g., (1) SEQ ID NOs:3-5 and SEQ ID NOs:6-8 (antibody P11); (2) SEQ ID NOs:11-13 and SEQ ID NOs:14-16 (antibody P17); (3) SEQ ID NOs:19-21 and SEQ ID NOs:22-24 (antibody P28); (4) SEQ ID NOs:27-29 and SEQ ID NOs:30-32 (antibody P130); (5) SEQ ID NOs:35-37 and SEQ ID NOs:38-40 (antibody P240); (6) SEQ ID NOs:43-45 and SEQ ID NOs:46-48 (antibody P1187); (7) SEQ ID NOs:51-53 and SEQ ID NOs:54-56 (antibody 1707); (8) SEQ ID NOs:59-61 and SEQ ID NOs:62-64:(antibody P1955); (9) SEQ ID NOs:67-69 and SEQ ID NOs:70-72 (antibody P2388); (10) SEQ ID NOs:75-77 and SEQ ID NOs:78-80 (antibody P2653); (11) SEQ ID NOs:83-85 and SEQ ID NOs:86-88:(antibody P2861); (12) SEQ ID NOs:91-93 and SEQ ID NOs:94-96 (antibody P3208); (13) SEQ ID NOs:99-101 and SEQ ID NOs:102-104 (antibody N277); (14) SEQ ID NOs:107-109 and SEQ ID NOs:110-112 (antibody N350); (15) SEQ ID NOs:115-117 and SEQ ID NOs:118-120 (antibody N484); (16) SEQ ID NOs:123-125 and SEQ ID NOs:126-128 (antibody N2282); (17) SEQ ID NOs:131-133 and SEQ ID NOs:134-136 (antibody N2430); or (18) SEQ ID NOs:139-141 and SEQ ID NOs:142-144 (antibody E2810). Still another exemplary pharmaceutical composition includes a dsFv fragment, which can include one or more modifications to the amino acid sequence as appropriate and understood by one of ordinary skill in the art.

In some embodiments, the compositions of the invention contain a carrier for the antibody, the antibody fragment, the antibody-based binding protein or the ADC, desirably a pharmaceutically acceptable carrier. The pharmaceutically acceptable carrier can be any suitable pharmaceutically acceptable carrier. It can be one or more compatible solid or liquid fillers, diluents, other excipients, or encapsulating substances which are suitable for administration into a human or veterinary patient (e.g., a physiologically acceptable carrier or a pharmacologically acceptable carrier). The term “carrier” denotes an organic or inorganic ingredient, natural or synthetic, with which the active ingredient is combined to facilitate the use of the active ingredient, e.g., the administration of the active ingredient to a subject. The pharmaceutically acceptable carrier can be co-mingled with one or more of the active components, e.g., a hybrid molecule, and with each other, when more than one pharmaceutically acceptable carrier is present in the composition, in a manner so as not to substantially impair the desired pharmaceutical efficacy. Pharmaceutically acceptable materials typically are capable of administration to a subject, e.g., a patient, without the production of significant undesirable physiological effects such as nausea, dizziness, rash, or gastric upset. It is, for example, desirable for a composition comprising a pharmaceutically acceptable carrier not to be immunogenic when administered to a human patient for therapeutic purposes.

Pharmaceutical compositions of the invention can additionally contain suitable buffering agents, including, for example, acetic acid in a salt, citric acid in a salt, boric acid in a salt, and phosphoric acid in a salt. The compositions can also optionally contain suitable preservatives, such as benzalkonium chloride, chlorobutanol, parabens, and thimerosal. Pharmaceutical compositions of the invention can be presented in unit dosage form and can be prepared by any suitable method, many of which are well known in the art of pharmacy. Such methods include the step of bringing the antibody of the invention into association with a carrier that constitutes one or more accessory ingredients. In general, the composition is prepared by uniformly and intimately bringing the active agent into association with a liquid carrier, a finely divided solid carrier, or both, and then, if necessary, shaping the product.

A composition suitable for parenteral administration conveniently comprises a sterile aqueous preparation of the inventive composition, which preferably is isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed, such as synthetic mono- or di-glycerides. In addition, fatty acids such as oleic acid can be used in the preparation of injectables. Carrier formulations suitable for oral, subcutaneous, intravenous, intramuscular, etc. administrations can be found in Remington's Pharmaceutical Sciences, Mack Publishing Co., Easton, PA.

Preparation of pharmaceutical compositions of the invention and their various routes of administration can be carried out in accordance with methods well known in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. The delivery systems useful in the context of the invention include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known to those of ordinary skill in the art. Suitable release delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and triglycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

The invention also provides kits suitable for carrying out the methods of the invention. Typically, the kits contain two or more components required for performing the therapeutic or diagnostic methods of the invention. Kit components include, but are not limited to, one or more antibodies, antibody-based binding proteins, antibody fragments thereof, or ADCs of the invention, appropriate reagents, and/or equipment. In some embodiments, the kits can contain an antibody, antibody-based binding protein, antibody fragment thereof, or ADC of the invention and an immunoassay buffer suitable for detecting PTPRG, Nectin-1, or endoglin (e.g. by ELISA, flow cytometry, magnetic sorting, or FACS). The kit may also contain one or more microtiter plates, standards, assay diluents, wash buffers, adhesive plate covers, magnetic beads, magnets, and/or instructions for carrying out a method of the invention using the kit. The kits can include an antibody, antibody-based binding protein, antibody fragment thereof of the invention bound to a substrate (e.g., a multi-well plate or a chip), which is suitably packaged and useful to detect PTPRG, Nectin-1, or endoglin. In some embodiments, the kits include an antibody, antibody-based binding protein, antibody fragment thereof of the invention that is conjugated to a label, such as, a fluorescent label, a biologically active enzyme label, a luminescent label, or a chromophore label. The kits can further include reagents for visualizing the conjugated antibody, antibody-based binding protein, antibody fragment thereof, e.g., a substrate for the enzyme. In some embodiments, the kits include an antibody or antigen-binding fragment of the invention that is conjugated to a contrast agent and, optionally, one or more reagents or pieces of equipment useful for imaging the antibody, antibody-based binding protein, antibody fragment thereof in a subject.

Generally the antibody, antibody-based binding protein, antibody fragment thereof or ADC of the invention in a kit is suitably packaged, e.g., in a vial, pouch, ampoule, and/or any container appropriate for a therapeutic or detection method. Kit components can be provided as concentrates (including lyophilized compositions), which may be further diluted prior to use, or they can be provided at the concentration of use. For use of the antibody of the invention in vivo, single dosages may be provided in sterilized containers having the desired amount and concentration of components.

### EXAMPLES

The following examples are provided to further illustrate the invention but not to limit its scope. Other variants of the invention will be readily apparent to one of ordinary skill in the art and are encompassed by the appended claims.

### Example 1. Antibody Discovery for Acute Myeloid Leukemia

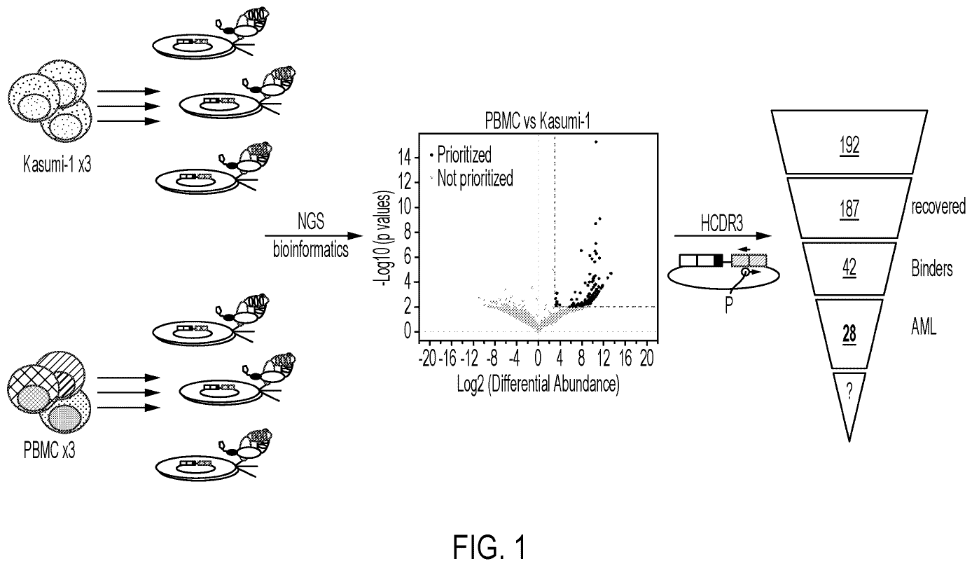

FIG. 1 is a schematic of the steps in antibody discovery for acute myeloid leukemia. One round of traditional whole cell panning, followed by another round of FBC (Fab-phage biotinylation & capture) panning, was performed in triplicates with both Kasumi-1 and PBMC cells. Output phages were then sequenced and analyzed to obtain hits specific for Kasumi-1 cells based on differential abundance and statistical significance (prioritized dots in the Volcano Plot). The corresponding mAbs (187) are then recovered using an around-the-horn PCR method. 42 of them were confirmed as AML binders, in which 28 only bind to AML cells but not PBMC.

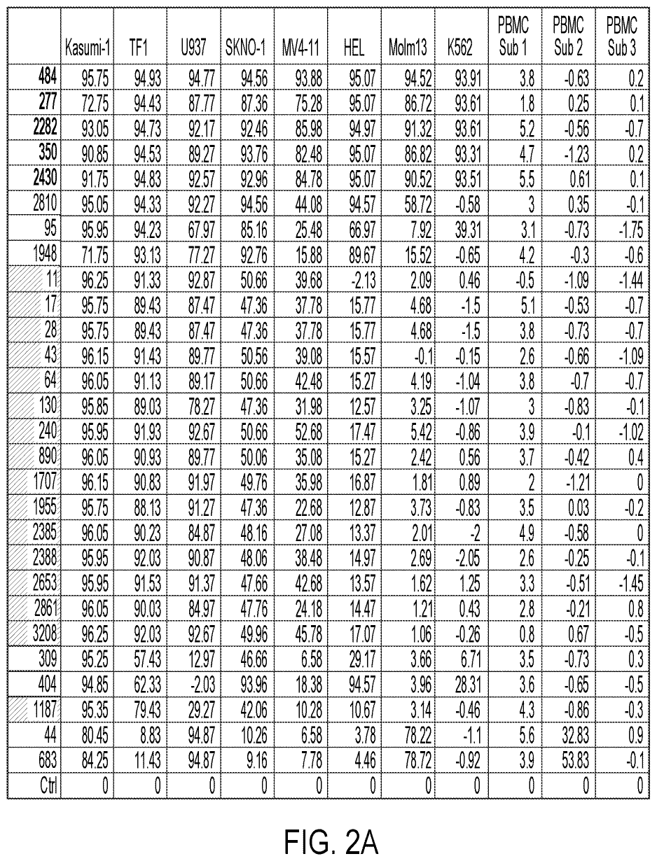

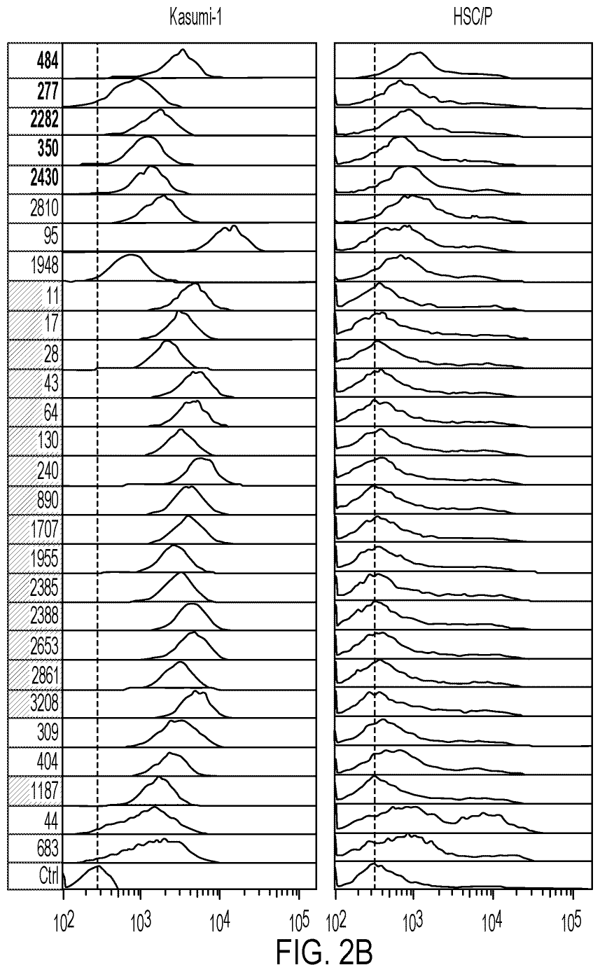

Those 28 mAbs were further analyzed by flow cytometry to determine their binding activity to different types of AML cell lines (Kasumi-1, TF1, U937, SKNO-1, MV4-11, HEL, Molm13, and K562), PBMC (PBMC Sub 1, PBMC Sub 2, and PBMC Sub 3), and HSC/P (Hematopoietic Stem and Progenitor Cells), and were classified into different groups based on the table as shown in FIG. 2A, and the flow cytometry profiles as shown in FIG. 2B. Clones in one group might recognize same antigen or epitope. While clones in boldface group bind both AML cell lines and HSC/P, the ones in angled-lines shading group only bind AML cell lines but not HSC/P. AML cell lines. FIG. 2A, left panel, is a table showing binding of antibodies to AML cell lines and PBMC from flow cytometry studies. FIG. 2B, shows results of flow cytometry profiles of binding of antibodies to Kasumi-1 cells (an AML cell line) and to HSC/P.

### Example 2. Target Convolution and Evaluation of mAb_11

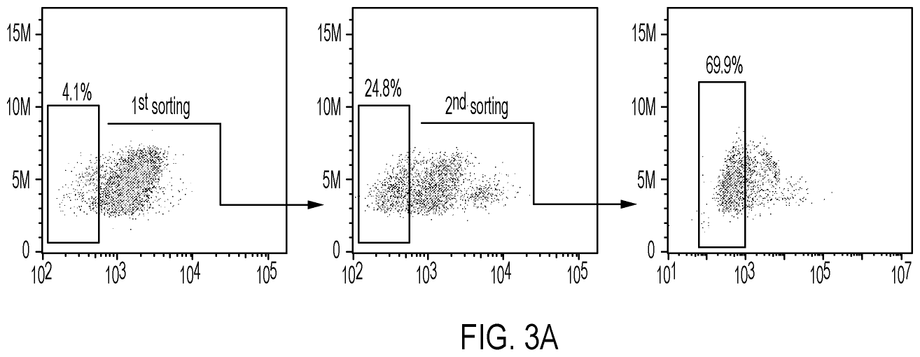

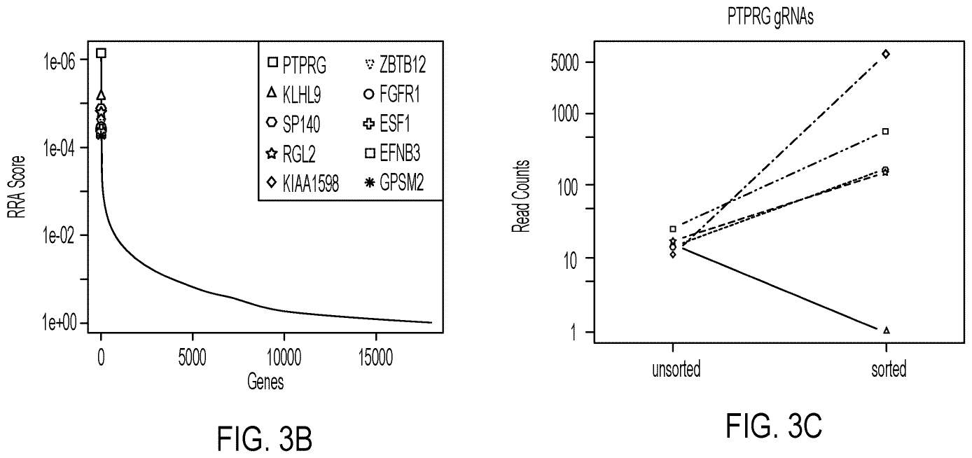

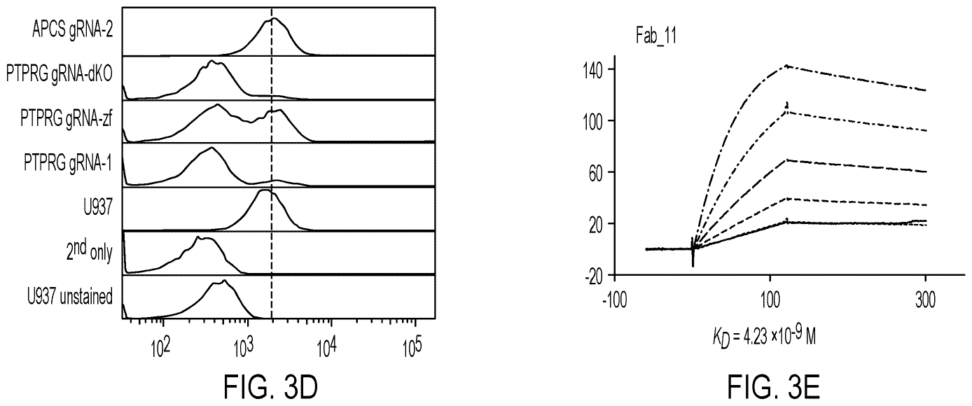

FIGS. 3A-C depict Target deconvolution of mAb_11 by screening of CRISPR library. 3A. HEL cells transduced with CRISPR gRNA library were sorted by IgG_11 to enrich antigen-negative cells due to gRNA-guided knockout by Cas9. After two rounds of sorting, 69.9% cells did not express the antigen anymore. 3B. Antigen-negative cells after sorting were collected to extract genomic DNA for PCR to amplify gRNA integrated into genome, followed by NGS and MAGeCK analysis to identify top gRNAs that could guide Cas9 to knock out the target antigen. PTPRG ranked top 1 for mAb_11. 3C. 4 out of 5 gRNAs targeting PTPRG had been significantly enriched. D. Top1 gRNA from FIG. 3C (marked as PTPRG gRNA-1) and another gRNA targeting PTPRG (PTPRG gRNA-zf) chosen randomly from one of the 8 gRNAs targeting PTPRG from a different CRISPR-gRNA library (GeCKO v2 library, Sanjana et al., supra) (marked as PTPRG gRNA-zf) were cloned and transduced, or co-transduced (marked as PTPRG gRNA-dKO, Top 1 gRNA from FIG. 3C and PTPRG gRNA-zf) into U937 cells, where Cas9-mediated KO of PTPRG could abolish (PTPRG gRNA-1) or diminish (PTPRG gRNA-zf) the binding of Fab_11 compared to parental U937 cells (marked as U937) APCS gRNA-2 is a negative control of gRNA targeting APCS protein randomly chosen from the CRISPR gRNA library of FIG. 3A. U937 cells treated with secondary antibody only (Jackson Immunoresearch, cat #: 109-116-097) (marked as 2^{nd} only), and U937 cells unstained (marked as unstained). E. SPR data showing the affinity of Fab_11 to human PTPRG extracellular domain (ECD) (residues 20~736 of human PTPRG) fused to hFc,(hPTPRG-ECD-hFc) (hPTRPG-ECD fused to hFc, produced in-house by transient transfection of Expi293 cells) (K_{D}=4.23×10⁻⁹ M)

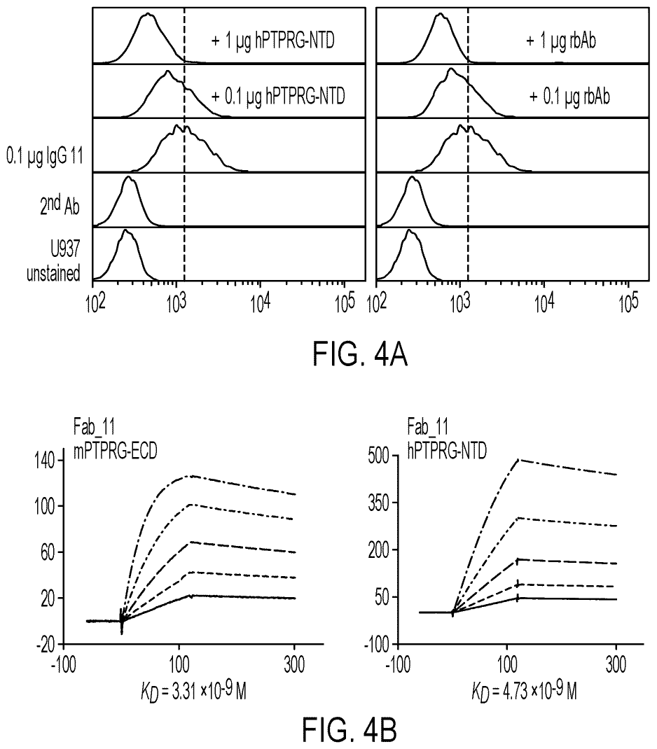

FIGS. 4A-B depict Target evaluation of mAb_11 by flow cytometry and SPR. 4A. The binding signals of IgG_11 could be diminished by either recombinant PTPRG protein (left panel) or rabbit polyclonal antibodies targeting human PTPRG (Invitrogen: Cat #PA5-89705) (right panel) in a concentration-dependent manner. (U937 cells treated with 0.1 μg IgG_11 only, marked as 0.1 μg IgG_11), (U937 cells treated with secondary antibody (Jackson Immunoresearch, cat #: 109-116-097) only (marked as 2nd Ab), (U937 cells unstained) 4B. SPR data showed Fab-11 was able to bind the extracellular domain (ECD) of mouse PTPRG ECD (amino acid residues 20-733 of mouse PTPRG) fused to hFc, produced in-house (mPTPRG-ECD), (K_{D}=3.31×10⁻⁹ M) and the N-terminal domain (NTD) of human PTPRG (human PTPRG NTD, amino acid residues 20~448 of human PTPRG), (K_{D}=4.73×10⁻⁹ M) with high affinities.

### Example 3. Target Deconvolution of Angled-Lines Shading Group (of FIGS. 2A and 2B) Clones Fab_11, Fab_17, Fab_28, Fab_64, Fab_130, Fab_240, Fab_1707, Fab_1955, Fab_2385 Fab_2388, Fab_2653, Fab_2861, Fab_3208

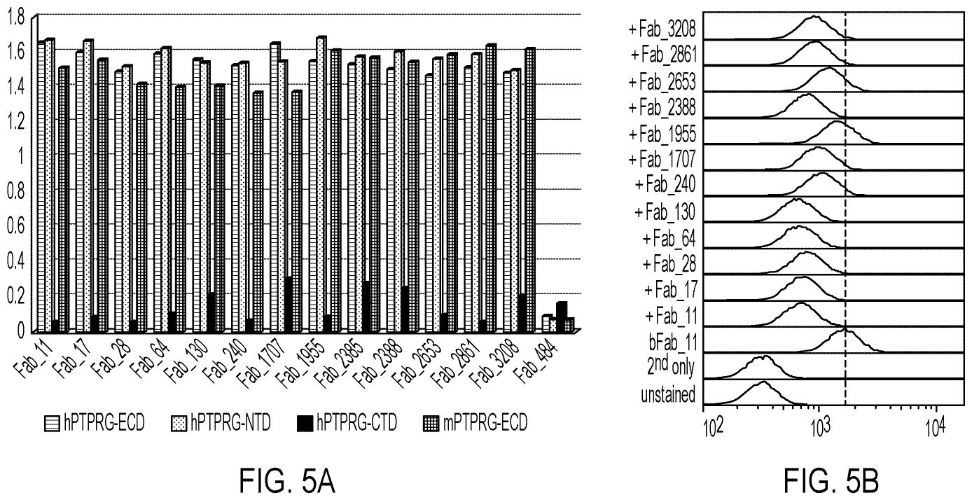

FIGS. 5A-B depict Target deconvolution of other clones in the angled-lines shading group by ELISA and flow cytometry. 5A. ELISA showed all the other clones in the angled-lines shading group recognized human and mouse PTPRG, specifically, N-terminal domain (NTD) of human PTPRG (human PTPRG NTD, amino acid residues 20~448 of human PTPRG), but not C-terminal domain (CTD) of human PTPRG (human PTPRG CTD, amino acid residues 349~736 of human PTPRG), Fab_484 (boldface group) shown for comparison. Mouse PTPRG-ECD-hFc indicated in FIG. 5A as mPTPRG-ECD. 5B. Epitope binning by flow cytometry showed all the other clones probably shared an epitope with mAb_11 as they (unbiotinylated Fab) competed with biotinylated Fab_11 to bind PTPRG on cell surface of U937 cells when detected by streptavidin-PE.

### Example 4. Target Deconvolution of mAb_1187, an Angled-Lines Shading Group Member that Also Recognizes PTPRG

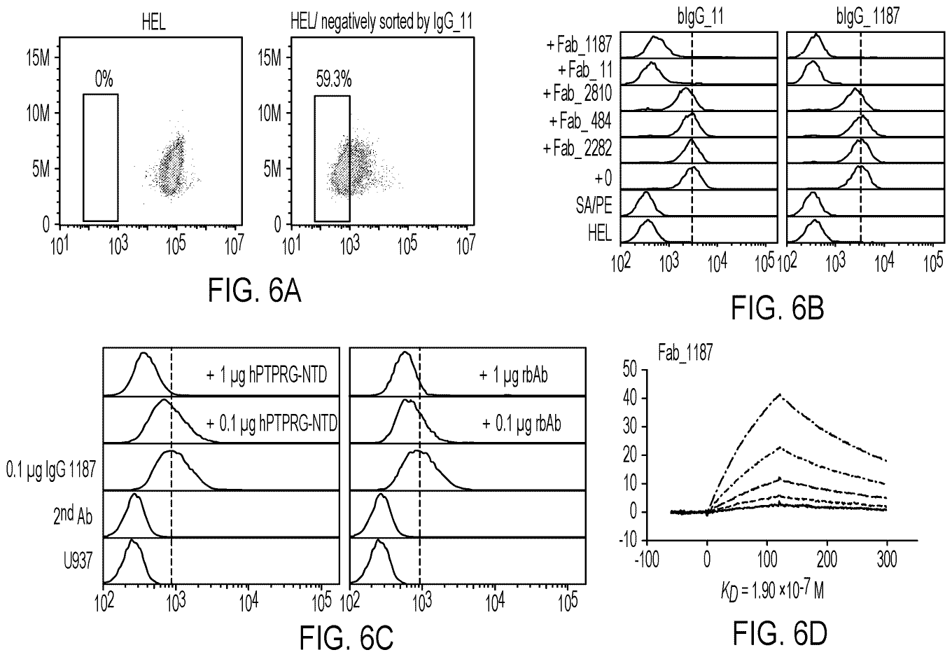

FIGS. 6A-D depict Target deconvolution of mAb_1187, which also recognizes PTPRG. 6A. HEL parental cells (left panel) or HEL cells negatively sorted by IgG_11 (right panel) were stained by biotinylated IgG_1187, while parental cells were 100% positive for IgG_1187, 59.3% of IgG_11 sorted HEL cells (69.9% negative for IgG_11 as shown in FIG. 3A) became negative for IgG_1187, indicating mAb_1187 may recognize same antigen PTPRG as mAb_11, or an antigen regulated by PTPRG. 6B. The binding signals of bIgG_11 and bIgG_1187 could be diminished by both Fab_11 and Fab_1187. 6C. The binding signals of IgG_1187 could be diminished by either recombinant PTPRG protein (left panel) or rabbit polyclonal antibodies targeting human PTPRG (Invitrogen: Cat #PA5-89705) (right panel) in a concentration-dependent manner. 6D. SPR data showing the affinity of Fab_1187 to (hPTPRG-ECD-hFc) (K_{D}=1.90×10⁻⁷ M)

### Example 5. Target Deconvolution of mAb_2282 in the Boldface Group (of FIGS. 2A and 2B) by Screening of CRISPR Library

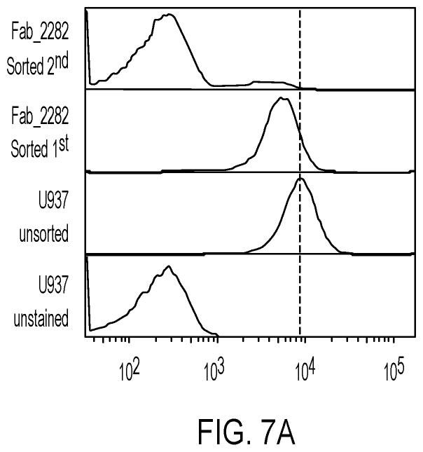

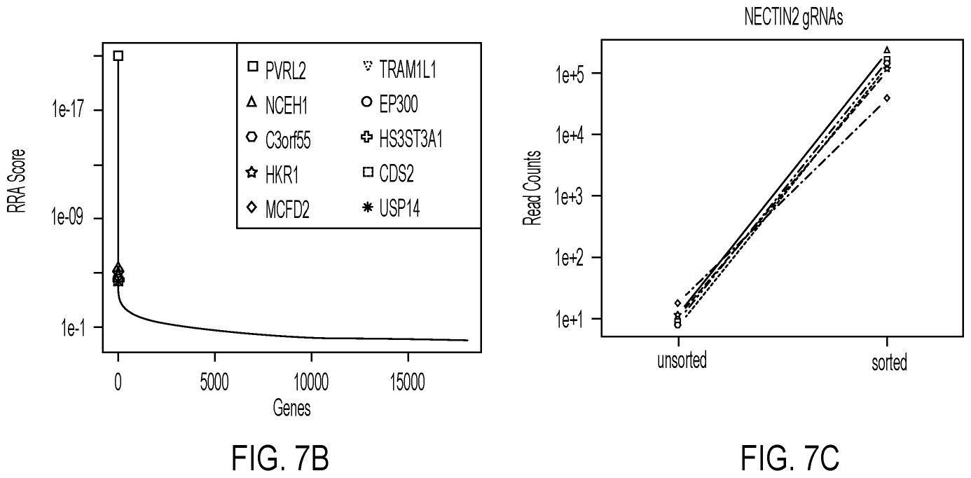

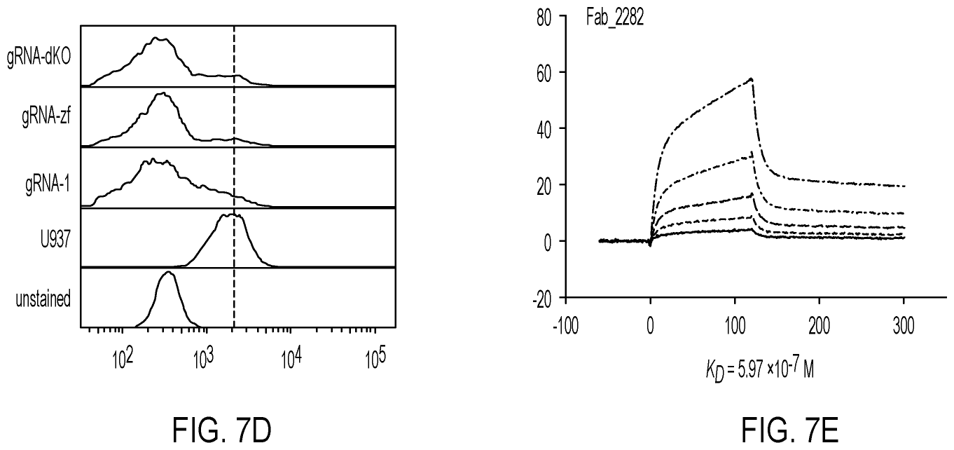

FIGS. 7A-E depict Target deconvolution of mAb_2282 in the boldface group by screening of CRISPR library. 7A. U937 cells transduced with CRISPR gRNA library were sorted by Fab_2282 to enrich antigen-negative cells due to gRNA-guided knockout by Cas9. After two rounds of sorting, most of the cells did not express the antigen anymore. 7B. Antigen-negative cells after sorting were collected to extract genomic DNA for PCR to amplify gRNA integrated into genome, followed by NGS and MAGeCK analysis to identify top gRNAs that could guide Cas9 to knock out the target antigen. PVRL2 (also named NECTIN2) ranked top 1 for mAb_2282. 7C. All 5 gRNAs targeting NECTIN2 had been significantly enriched. 7D. Top1 gRNA from FIG. 7C (marked as gRNA-1) and another gRNA targeting NECTIN2 (gRNA-zf) from a different CRISPR-gRNA library (GeCKO v2 library, Sanjana et al., supra) (marked as gRNA-zf), were cloned and transduced, or co-transduced (marked as gRNA-dKO, top 1 gRNA from FIG. 7C and gRNA-zf) into U937 cells, where Cas9-mediated KO of NECTIN2 could abolish the binding of Fab_2282 compared to parental U937 cells (marked as U937). 7E. SPR data showing the affinity of Fab_2282 to human NECTIN2-ECD-hFc (K_{D}=5.97×10⁻⁷ M) (human Nectin-2 extracellular domain (amino acid residues 1-360 of human Nectin-2) fused to hFc (hNectin-2-ECD-hFc, Sino biologics, cat #10005-H02H).

### Example 6. Target Deconvolution of Other Clones in the Boldface Group (of FIGS. 2A and 2B) by Competition Flow Cytometry

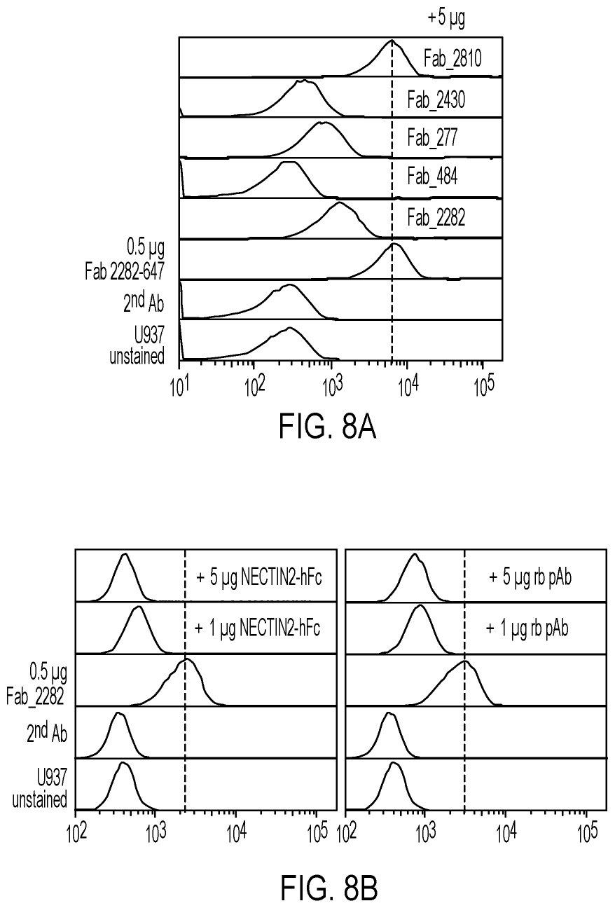

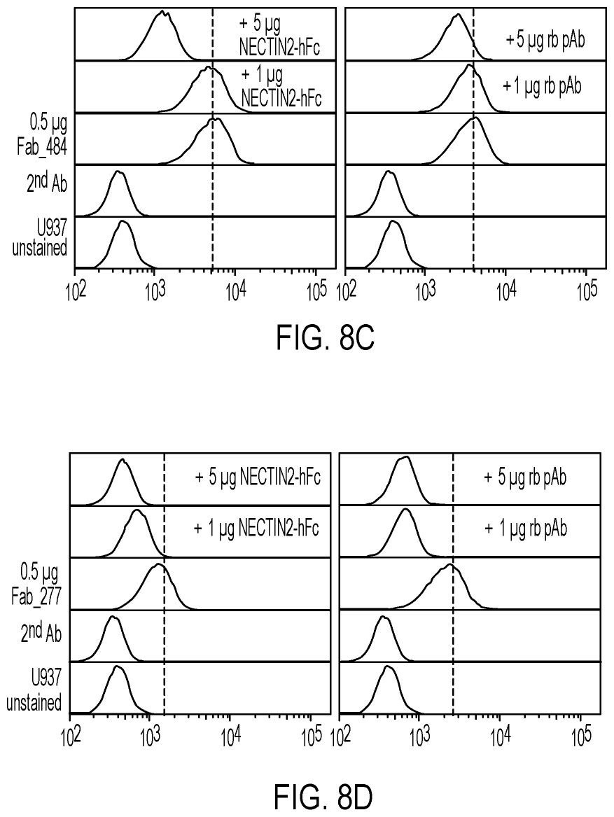

FIGS. 8A-D depict Target deconvolution of other clones in the boldface group by competition flow cytometry. 8A. The binding signals of Fab_2282 (labeled with Alexa 647) to U937 cells could be diminished by Fab_2282 itself, Fab_484, Fab_277, and Fab_2430, but not Fab_2810, indicating Fab_2282, Fab_484, Fab_277, and Fab_2430 may recognize the same antigen or epitope. 8B-8D. The binding signals of Fab_2282 (8B), Fab_484 (8C), and Fab_277 (8D) could be diminished by either recombinant Nectin-2 ECD-hFc protein (human Nectin-2 extracellular domain (amino acid residues 1-360 of human Nectin-2) fused to hFc (hNectin-2-ECD-hFc, Sino biologics, cat #10005-H02H) (left panel) or rabbit polyclonal antibodies targeting human NECTIN2 (Sino biologics: 10005-RP03) (right panel) in a concentration-dependent manner.

### Example 7. Target Deconvolution of Other Clones in the Boldface Group (of FIGS. 2A and 2B) by CRISPR KO and SPR

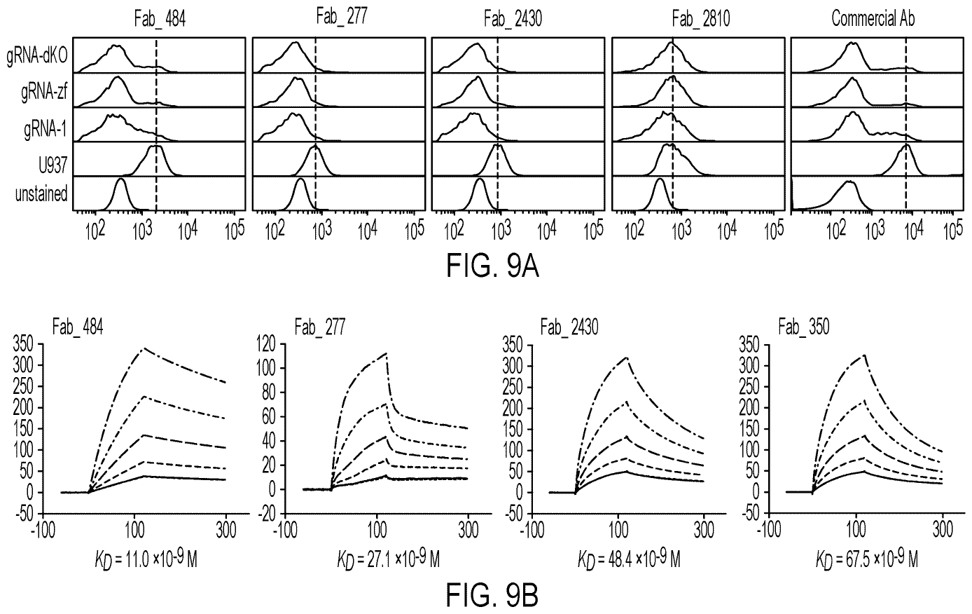

FIG. 9 depicts Target deconvolution of other clones in the boldface group by CRISPR KO and SPR. A. Top1 gRNA as in FIG. 7C (marked as gRNA-1) and another gRNA targeting NECTIN2 (gRNA-zf) from a different CRISPR-gRNA library (GeCKO v2 library, Sanjana et al., supra) (marked as gRNA-zf), were cloned and transduced, or co-transduced (marked as gRNA-dKO, top 1 gRNA as in FIG. 7C and gRNA-zf) into U937 cells, where Cas9 could abolish the binding of Fab_484, Fab_277, Fab_2430, and a commercial mAb targeting NECTIN2 (Biolegend: 337411), but not control Fab_2810, compared to parental U937 cells (marked as U937). B. SPR data showing the affinities of Fab_484, Fab_277, Fab_2430, and Fab_350 to hNectin-2-ECD-hFc.

### Example 8. Target Deconvolution and Evaluation of mAb_2810

FIGS. 10A-E depicts Target deconvolution of mAb_2810 by screening of CRISPR library. 10A. U937 cells transduced with CRISPR gRNA library were sorted by Fab_2810 to enrich antigen-negative cells due to gRNA-guided knockout by Cas9. After two rounds of sorting, most of the cells did not express the antigen anymore. 10B. Antigen-negative cells after sorting were collected to extract genomic DNA for PCR to amplify gRNA integrated into genome, followed by NGS and MAGeCK analysis to identify top gRNAs that could guide Cas9 to knock out the target antigen. END (or CD105) ranked top 1 for mAb_2810. 10C. All 5 gRNAs targeting CD105 had been significantly enriched. 10D. Top1 gRNA from FIG. 10C (marked as gRNA-1) and another gRNA targeting CD105 (gRNA-zf) from a different CRISPR-gRNA library (GeCKO v2 library, Sanjana et al., supra) (marked as gRNA-zf), were cloned and transduced, or co-transduced (top1 gRNA from FIG. 10C and gRNA-zf, marked as gRNA-dKO) into U937 cells, where Cas9-mediated KO of CD105 could abolish the binding of Fab_2810 compared to parental U937 cells (marked as U937). The gRNA-zf targeting CD105 did not appear to knockout CD105, so Fab-2810 binding signal was not diminished in presence of gRNA-zf alone (marked as gRNA-zf) 10E. SPR data showing the affinity of Fab_2810 to human CD105-ECD: amino acid residues Met 1-Gly 586 of human CD105 (Sino bio: Cat: 10149-H02H) (K_{D}=8.22×10⁻⁹ M)

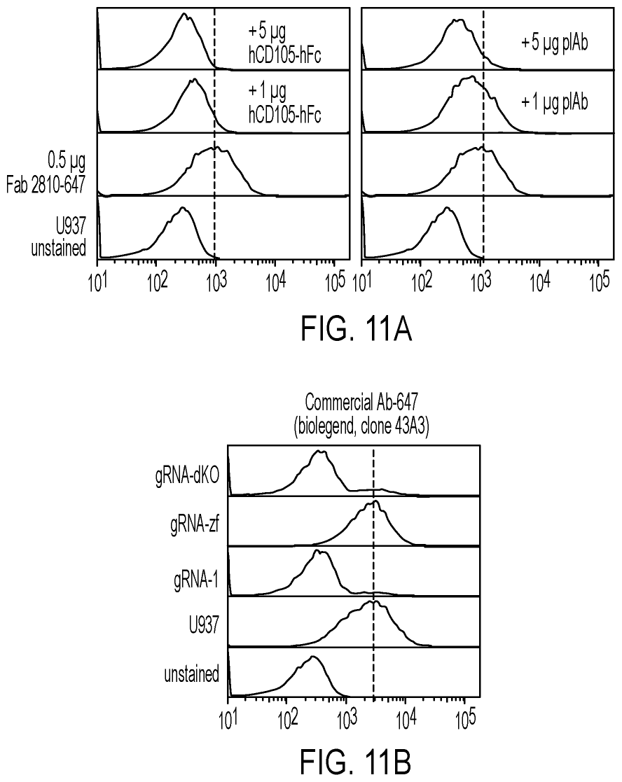

FIGS. 11A-B depict Target evaluation of mAb_2810 by competition flow cytometry and CRISPR KO. 11A. The binding signals of Fab_2810 could be diminished by either recombinant human CD105 (hCD105-ECD-hFc; Sino bio: Cat: 10149-H02H)) protein (left panel) or goat polyclonal antibody targeting human CD105 (R&D, cat #AF1097) (right panel) in a concentration-dependent manner. 11B. Top1 gRNA as in FIG. 10C (marked as gRNA-1) and another gRNA targeting CD105 as in FIG. 10D (gRNA-zf) from a different CRISPR-gRNA library (GeCKO v2 library, Sanjana et al., supra) (marked as gRNA-zf), were cloned and transduced, or co-transduced (marked as gRNA-dKO, top1 gRNA from FIG. 10C and gRNA-zf) into U937 cells, where Cas9-mediated KO of CD105 could abolish the binding of a commercial mAb targeting CD105 (Alexa Fluor 647 anti-human CD105, clone 43A3, Biolegend cat #323212) compared to parental U937 cells (marked as U937). The gRNA-zf targeting CD105 did not appear to knockout CD105, so commercial mAb targeting CD105 binding signal was not diminished in presence of gRNA-zf alone (marked as gRNA-zf).

### Example 9. Specificity and Affinity of New mAbs to the Corresponding Antigens

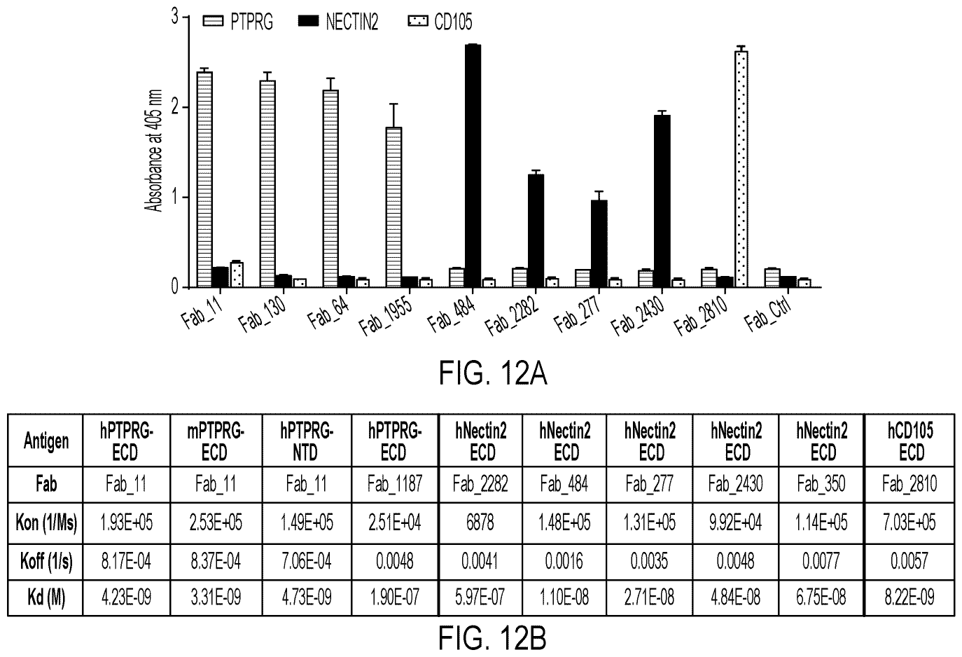

FIGS. 12A-B depict Specificity and affinity of new mAbs to the corresponding antigens. 12A. ELISA data showed that all the new mAbs specifically recognized their cognate antigens (human PTPRG-ECD, human Nectin-2 ECD, human CD105-ECD) without cross-reactivity. Control (Fab-Ctrl) is Fab_402 anti-human ROR 1. 12B. Summary of affinities for each antibody

### Example 10. Amino Acid Sequences of Rabbit Variable Domains (V_{H} and V_{λ},) of Chimeric Rabbit/Human Fab Clones

FIGS. 13A-D depict an amino acid sequence alignment of the rabbit variable domains (V_{H} and V_{λ},) of chimeric rabbit/human Fab clones is shown with framework regions (FR) and complementarity determining regions (CDR) using Kabat numbering. CDRs shown are of Kabat definition. Clones starting with P bind PTPRG, clones starting with N bind Nectin-2, and clone starting with E binds CD105/endoglin. Table 2 shows SEQ ID NOs of rabbit variable domains (V_{H} and V_{λ},) of chimeric rabbit/human Fab clones, and Kabat definition CDRs. 13A-13B (V_{H}); 13C-13D (V_{λ})

TABLE 2
SEQ ID NOs of rabbit variable domains (V_{H} and V_{λ},)
of chimeric rabbit/human Fab clones, and Kabat definition CDRs

| Clone | V_{H} | CDR-H1 | CDR-H2 | CDR-H3 | V_{λ} | CDR-L1 | CDR-L2 | CDR-L3 |
|---|---|---|---|---|---|---|---|---|
| P11 | 1 | 3 | 4 | 5 | 2 | 6 | 7 | 8 |
| P17 | 9 | 11 | 12 | 13 | 10 | 14 | 15 | 16 |
| P28 | 17 | 19 | 20 | 21 | 18 | 22 | 23 | 24 |
| P130 | 25 | 27 | 28 | 29 | 26 | 30 | 31 | 32 |
| P240 | 33 | 35 | 36 | 37 | 34 | 38 | 39 | 40 |
| P1187 | 41 | 43 | 44 | 45 | 42 | 46 | 47 | 48 |
| P1707 | 49 | 51 | 52 | 53 | 50 | 54 | 55 | 56 |
| P1955 | 57 | 59 | 60 | 61 | 58 | 62 | 63 | 64 |
| P2388 | 65 | 67 | 68 | 69 | 66 | 70 | 71 | 72 |
| P2653 | 73 | 75 | 76 | 77 | 74 | 78 | 79 | 80 |
| P2861 | 81 | 83 | 84 | 85 | 82 | 86 | 87 | 88 |
| P3208 | 89 | 91 | 92 | 93 | 90 | 94 | 95 | 96 |
| N277 | 97 | 99 | 100 | 101 | 98 | 102 | 103 | 104 |
| N350 | 105 | 107 | 108 | 109 | 106 | 110 | 111 | 112 |
| N484 | 113 | 115 | 116 | 117 | 114 | 118 | 119 | 120 |
| N2282 | 121 | 123 | 124 | 125 | 122 | 126 | 127 | 128 |
| N2430 | 129 | 131 | 132 | 133 | 130 | 134 | 135 | 136 |
| E2810 | 137 | 139 | 140 | 141 | 138 | 142 | 143 | 144 |

### REFERENCES

1. The Antibody Society. Therapeutic monoclonal antibodies approved or in review in the EU or US. [cited 2022 April 19]. Available from: WorldWideWeb antibodysociety.org/resources/approved-antibodies.
2. McCafferty J, Griffiths A D, Winter G, Chiswell D J. Phage antibodies: filamentous phage displaying antibody variable domains. Nature. 1990; 348(6301):552-4. doi: 10.1038/348552a0. PubMed PMID: 2247164.
3. Barbas C F, 3rd, Kang A S, Lerner R A, Benkovic S J. Assembly of combinatorial antibody libraries on phage surfaces: the gene III site. Proc Natl Acad Sci USA. 1991; 88(18):7978-82. doi: 10.1073/pnas.88.18.7978. PubMed PMID: 1896445.
4. Frenzel A, Schirrmann T, Hust M. Phage display-derived human antibodies in clinical development and therapy. mAbs. 2016; 8(7):1177-94. doi: 10.1080/19420862.2016.1212149. PubMed PMID: 27416017.
5. Lerner R A. Combinatorial antibody libraries: new advances, new immunological insights. Nat Rev Immunol. 2016; 16(8):498-508. doi: 10.1038/nri.2016.67. PubMed PMID: 27374636.
6. Alfaleh M A, Jones M L, Howard C B, Mahler S M. Strategies for Selecting Membrane Protein-Specific Antibodies using Phage Display with Cell-Based Panning. Antibodies (Basel). 2017; 6(3). doi: 10.3390/antib6030010. PubMed PMID: 31548525.
7. Hagemann U B, Gunnarsson L, Geraudie S, Scheffler U, Griep R A, Reiersen H, Duncan A R, Kiprijanov S M. Fully human antagonistic antibodies against CCR4 potently inhibit cell signaling and chemotaxis. PLoS One. 2014; 9(7):e103776. doi: 10.1371/journal.pone.0103776. PubMed PMID: 25080123.
8. Siegel D L, Chang T Y, Russell S L, Bunya V Y. Isolation of cell surface-specific human monoclonal antibodies using phage display and magnetically-activated cell sorting: applications in immunohematology. J Immunol Methods. 1997; 206(1-2):73-85. doi: 10.1016/s0022-1759(97)00087-2. PubMed PMID: 9328570.
9. Fransson J, Tornberg U C, Borrebaeck C A, Carlsson R, Frendéus B. Rapid induction of apoptosis in B-cell lymphoma by functionally isolated human antibodies. International journal of cancer. 2006; 119(2):349-58. doi: 10.1002/ijc.21829. PubMed PMID: 16477633.
10. Baskar S, Suschak J M, Samija I, Srinivasan R, Childs R W, Pavletic S Z, Bishop M R, Rader C. A human monoclonal antibody drug and target discovery platform for B-cell chronic lymphocytic leukemia based on allogeneic hematopoietic stem cell transplantation and phage display. Blood. 2009; 114(20):4494-502. doi: 10.1182/blood-2009-05-222786. PubMed PMID: 19667400.
11. Hosen N, Matsunaga Y, Hasegawa K, Matsuno H, Nakamura Y, Makita M, Watanabe K, Yoshida M, Satoh K, Morimoto S, Fujiki F, Nakajima H, Nakata J, Nishida S, Tsuboi A, Oka Y, Manabe M, Ichihara H, Aoyama Y, Mugitani A, Nakao T, Hino M, Uchibori R, Ozawa K, Baba Y, Terakura S, Wada N, Morii E, Nishimura J, Takeda K, Oji Y, Sugiyama H, Takagi J, Kumanogoh A. The activated conformation of integrin p7 is a novel multiple myeloma-specific target for CAR T cell therapy. Nat Med. 2017; 23:1436. doi: 10.1038/nm.4431
12. Bull C, Stoel M A, den Brok M H, Adema G J. Sialic acids sweeten a tumor's life. Cancer Res. 2014; 74(12):3199-204. doi: 10.1158/0008-5472.Can-14-0728. PubMed PMID: 24830719.
13. Flynn R A, Pedram K, Malaker S A, Batista P J, Smith B A H, Johnson A G, George B M, Majzoub K, Villalta P W, Carette J E, Bertozzi C R. Small RNAs are modified with N-glycans and displayed on the surface of living cells. Cell. 2021; 184(12):3109-24.e22. doi: 10.1016/j.cell.2021.04.023. PubMed PMID: 34004145.
14. Bausch-Fluck D, Goldmann U, Muller S, van Oostrum M, Müller M, Schubert O T, Wollscheid B. The in silico human surfaceome. Proc Natl Acad Sci USA. 2018; 115(46):E10988-e97. doi: 10.1073/pnas.1808790115. PubMedPMID: 30373828.
15. Hoogenboom H R, de Bruine A P, Hufton S E, Hoet R M, Arends J W, Roovers R C. Antibody phage display technology and its applications. Immunotechnology. 1998; 4(1):1-20. doi: 10.1016/s1380-2933(98)00007-4. PubMed PMID: 9661810.
16. Derda R, Tang S K, Li S C, Ng S, Matochko W, Jafari M R. Diversity of phage-displayed libraries of peptides during panning and amplification. Molecules. 2011; 16(2):1776-803. doi: 10.3390/molecules16021776. PubMedPMID: 21339712.
17. Freeth J, Soden J. New Advances in Cell Microarray Technology to Expand Applications in Target Deconvolution and Off-Target Screening. SLAS Discov. 2020; 25(2):223-30. doi: 10.1177/2472555219897567. PubMed PMID: 31885307.
18. Huston-Paterson D J B S, Doranz B J. A High-Throughput Platform for Identifying Membrane Protein Antibody Targets. Genetic Engineering & Biotechnology News. 2016 Sep. 1.
19. Chang J, Peng H, Shaffer B C, Baskar S, Wecken I C, Cyr M G, Martinez G J, Soden J, Freeth J, Wiestner A, Rader C. Siglec-6 on Chronic Lymphocytic Leukemia Cells Is a Target for Post-Allogeneic Hematopoietic Stem Cell Transplantation Antibodies. Cancer Immunol Res. 2018; 6(9):1008-13. doi: 10.1158/2326-6066.Cir-18-0102. PubMed PMID: 29980538.
20. Wilson H D, Li X, Peng H, Rader C. A Sortase A Programmable Phage Display Format for Improved Panning of Fab Antibody Libraries. J Mol Biol. 2018; 430(21):4387-400. doi: 10.1016/j.jmb.2018.09.003. PubMed PMID: 30213726.
21. Glanville J, D'Angelo S, Khan T A, Reddy S T, Naranjo L, Ferrara F, Bradbury A R M. Deep sequencing in library selection projects: what insight does it bring?Current Opinion in Structural Biology. 2015; 33:146-60. doi:
22. Ferrara F, Teixeira A A, Naranjo L, Erasmus M F, D'Angelo S, Bradbury A R M. Exploiting next-generation sequencing in antibody selections—a simple PCR method to recover binders. mAbs. 2020; 12(1):1701792. doi: 10.1080/19420862.2019.1701792. PubMed PMID: 31829073.
23. Rouet R, Jackson K J L, Langley D B, Christ D. Next-Generation Sequencing of Antibody Display Repertoires. Front Immunol. 2018; 9:118. doi: 10.3389/fimmu.2018.00118. PubMed PMID: 29472918.
24. Jones M L, Alfaleh M A, Kumble S, Zhang S, Osborne G W, Yeh M, Arora N, Hou J J C, Howard C B, Chin D Y, Mahler S M. Targeting membrane proteins for antibody discovery using phage display. Scientific reports. 2016; 6:26240. doi: 10.1038/srep26240
25. Zhang H, Torkamani A, Jones T M, Ruiz D I, Pons J, Lerner R A. Phenotype-information-phenotype cycle for deconvolution of combinatorial antibody libraries selected against complex systems 2011; 108(33):13456-61. doi: 10.1073/pnas.1111218108% J Proceedings of the National Academy of Sciences.
26. Deschaght P, Vintem A P, Logghe M, Conde M, Felix D, Mensink R, Gongalves J, Audiens J, Bruynooghe Y, Figueiredo R, Ramos D, Tanghe R, Teixeira D, Van de Ven L, Stortelers C, Dombrecht B. Large Diversity of Functional Nanobodies from a Camelid Immune Library Revealed by an Alternative Analysis of Next-Generation Sequencing Data 2017; 8(420). doi: 10.3389/fimmu.2017.00420.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

Sequence Listing
amino acid sequence of mature heavy chain variable region of clone P11
SEQ ID NO: 1
AQSLEESGGRLVTPGTPLTLTCTVSGFSLSSYAMTWVRQAPGEGLEWIGTIGAGGSP
YYASWAKGRFTISKTATTVNLKIISPTTEDTATYFCAREGGGSYYFDLWGPGTLVTVSS
amino acid sequence of mature light chain variable region of clone P11
SEQ ID NO: 2
SFVLTQPASVQVNLGQTVSLTCTADTLSRSYASWYQQKPGQAPVLLIYRDTSRPSGV
PDRFSGSSSGNTATLTISGAQAGDEADYYCATSDGSGSTFHFVFGGGTQLTVTG
P11 Kabat CDRH1 amino acid sequence
SEQ ID NO: 3
SYAMT
P11 Kabat CDRH2 amino acid sequence
SEQ ID NO: 4
TIGAGGSPYYASWAKG
P11 Kabat CDRH3 amino acid sequence
SEQ ID NO: 5
EGGGSYYFDL
P11 Kabat CDRL1 amino acid sequence
SEQ ID NO: 6
TADTLSRSYAS
P11 Kabat CDRL2 amino acid sequence
SEQ ID NO: 7
RDTSRPS
P11 Kabat CDRL3 amino acid sequence
SEQ ID NO: 8
ATSDGSGSTFHFV
amino acid sequence of mature heavy chain variable region of clone P17
SEQ ID NO: 9
AQSLEEFGGRLATPGTPLTLTCTASGFSLGTYSMSWVRQAPGKGLEWIGTIGGRGDA
YYASWAKGRFTISRTSTTVDLKMTSLATEDTATYFCVRENYGYAFDIWGPGTLVTISS
amino acid sequence of mature light chain variable region of clone P17
SEQ ID NO: 10
SFVLTQPASVQVNLGQTVSLTCTADTLSRRYASWYQQKPGQAPVLLIYRDTSRPSGV
PDRFSGSSSGNTATLTISGAQAGDEADYYCATSDGSGSSYQCVFGGGTQLTVTG
P17 Kabat CDRH1 amino acid sequence
SEQ ID NO: 11
TYSMS
P17 Kabat CDRH2 amino acid sequence
SEQ ID NO: 12
TIGGRGDAYYASWAKG
P17 Kabat CDRH3 amino acid sequence
SEQ ID NO: 13
ENYGYAFDI
P17 Kabat CDRL1 amino acid sequence
SEQ ID NO: 14
TADTLSRRYAS
P17 Kabat CDRL2 amino acid sequence
SEQ ID NO: 15
RDTSRPS
P17 Kabat CDRL3 amino acid sequence
SEQ ID NO: 16
ATSDGSGSSYQCV
amino acid sequence of mature heavy chain variable region of clone P28
SEQ ID NO: 17
AQSVEESGGRLVTPGIPLTLTCTVSGFSLNNYAMSWVRQAPGKGLEWIGMIDSGGAA
YYATWARGRFTISRTSTTVDLKITSPTTEDTATYFCARELSSLYGAFDPWGPGTLVTVSS
amino acid sequence of mature light chain variable region of clone P28
SEQ ID NO: 18
SFVLTQPASVQVNLGQTVSLTCTADTLSRRYASWYQQKPGQAPVLLIYRDTSRPSGV
PDRFSGSSSGNTATLTISGAQAGDEADYYCATSDGSGSSYQCVFGGGTQLTVTG
P28 Kabat CDRH1 amino acid sequence
SEQ ID NO: 19
NYAMS
P28 Kabat CDRH2 amino acid sequence
SEQ ID NO: 20
MIDSGGAAYYATWARG
P28 Kabat CDRH3 amino acid sequence
SEQ ID NO: 21
ELSSLYGAFDP
P28 Kabat CDRL1 amino acid sequence
SEQ ID NO: 22
TADTLSRRYAS
P28 Kabat CDRL2 amino acid sequence
SEQ ID NO: 23
RDTSRPS
P28 Kabat CDRL3 amino acid sequence
SEQ ID NO: 24
ATSDGSGSSYQCV
amino acid sequence of mature heavy chain variable region of clone P130
SEQ ID NO: 25
QEQQKESGGGLFKPTDTLTLTCTVSGFSLSSNAISWVRQAPGNGLEWIGAI
DNSGSAYYATWAKGRSTITRATNLNTVTLKMTSVTAADTATYFCAREDVRADSIDL
NMWGPGTLVTISS
amino acid sequence of mature light chain variable region of clone P130
SEQ ID NO: 26
SFVLTQPASVQVNLGQTVSLTCTADTLSRSYASWYQQKPGQAPVLLIYRDTSRPSGV
PDRFSGSSSGNTATLTISGAQAGDEADYYCATSDGSGSSYQFVFGGGTQLTVTG
P130 Kabat CDRH1 amino acid sequence
SEQ ID NO: 27
SNAIS
P130 Kabat CDRH2 amino acid sequence
SEQ ID NO: 28
AIDNSGSAYYATWAKG
P130 Kabat CDRH3 amino acid sequence
SEQ ID NO: 29
EDVRADSIDLNM
P130 Kabat CDRL1 amino acid sequence
SEQ ID NO: 30
TADTLSRSYAS
P130 Kabat CDRL2 amino acid sequence
SEQ ID NO: 31
RDTSRPS
P130 Kabat CDRL3 amino acid sequence
SEQ ID NO: 32
ATSDGSGSSYQFV
amino acid sequence of mature heavy chain variable region of clone P240
SEQ ID NO: 33
AQSLEESGGRLVTPGTPLTLTCTVSGFSLSAYAMSWVRQAPGKGLEWIGIISSYGNTY
YATWAKGRFTISKTSTTVDLKITSPTTEDTATYFCARGDYGNGMSIWGPGTLVTVSS
amino acid sequence of mature light chain variable region of clone P240
SEQ ID NO: 34
SFVLTQPASVQVNLGQTVSLTCTADTLSRRYASWYQQKPGQAPVLLIYRDTRRPSGV
PDRFSGSSSGNTATLTISGAQAGDEADYYCATSDGSGSSYQCVFGGGTQLTVTG
P240 Kabat CDRH1 amino acid sequence
SEQ ID NO: 35
AYAMS
P240 Kabat CDRH2 amino acid sequence
SEQ ID NO: 36
IISSYGNTYYATWAKG
P240 Kabat CDRH3 amino acid sequence
SEQ ID NO: 37
GDYGNGMSI
P240 Kabat CDRL1 amino acid sequence
SEQ ID NO: 38
TADTLSRRYAS
P240 Kabat CDRL2 amino acid sequence
SEQ ID NO: 39
RDTRRPS
P240 Kabat CDRL3 amino acid sequence
SEQ ID NO: 40
ATSDGSGSSYQCV
amino acid sequence of mature heavy chain variable region of clone P1187
SEQ ID NO: 41
AQSVEESGGRLVTPGTPLTLTCTVSGIDLSSYAMSWVRQAPGKGLEWIGVIDGSGST
WYANWVKGRFTISKTTTVDLKITSPTTEDTATYFCAREDYGSETIDFNIWGPGTLVTVSS
amino acid sequence of mature light chain variable region of clone P1187
SEQ ID NO: 42
SFVLTQPASVQVNLGQTVSLTCTADTLSRRYASWYQQKPGQAPVLLIYRDTRRPSGV
PDRFSGSSSGNTATLTISGAQAGDEADYYCATSDGSGSSYQYVFGGGTQLTVTG
P1187 Kabat CDRH1 amino acid sequence
SEQ ID NO: 43
SYAMS
P1187 Kabat CDRH2 amino acid sequence
SEQ ID NO: 44
VIDGSGSTWYANWVKG
P1187 Kabat CDRH3 amino acid sequence
SEQ ID NO: 45
EDYGSETIDENI
P1187 Kabat CDRL1 amino acid sequence
SEQ ID NO: 46
TADTLSRRYAS
P1187 Kabat CDRL2 amino acid sequence
SEQ ID NO: 47
RDTRRPS
P1187 Kabat CDRL3 amino acid sequence
SEQ ID NO: 48
ATSDGSGSSYQYV
amino acid sequence of mature heavy chain variable region of clone P1707
SEQ ID NO: 49
AQSLEEFGGGLIKPGGTLTLTCTASGFTISSYDMSWVRQAPGSGLEWIGTMAYGGDT
YHASWAKSRSTITRDTNLNTVTLKMTSLTVADTATYFCARDVGSGHYYDDMYLWG
PGTLVTVSS
amino acid sequence of mature light chain variable region of clone P1707
SEQ ID NO: 50
SFVLTQPASVQVNFGQTVSLTCTADTLSRRYASWYQQKPGQAPVLLIYRDTSRPSGV
PDRFSGSSSGNTATLTISGAQAGDEADYYCATGDGSGSSYNYVFGGGTQLTVTG
P1707 Kabat CDRH1 amino acid sequence
SEQ ID NO: 51
SYDMS
P1707 Kabat CDRH2 amino acid sequence
SEQ ID NO: 52
TMAYGGDTYHASWAKS
P1707 Kabat CDRH3 amino acid sequence
SEQ ID NO: 53
DVGSGHYYDDMYL
P1707 Kabat CDRL1 amino acid sequence
SEQ ID NO: 54
TADTLSRRYAS
P1707 Kabat CDRL2 amino acid sequence
SEQ ID NO: 55
RDTSRPS
P1707 Kabat CDRL3 amino acid sequence
SEQ ID NO: 56
ATGDGSGSSYNYV
amino acid sequence of mature heavy chain variable region of clone P1955
SEQ ID NO: 57
AQSLEESGGRLVTPGTPLTLTCTASGFSLYSYDMSWVRQAPGKGLEWIGLIDGGGNT
YYASWAKGRFTISKTSSTTVDLKMTSLTTEDTATYFCARELWPAYSSGAFNIWGPGT
LVTISS
amino acid sequence of mature light chain variable region of clone P1955
SEQ ID NO: 58
SFVLTQPASVQVNLGQTVSLTCTADTLSRRYASWYQQKPGQAPVLLIYRDTRRPSGV
PDRFSGSSSGNTATLTISGAQAGDEADYYCVTTDGSGSSYLVVFGGGTQLTVTG
P1955 Kabat CDRH1 amino acid sequence
SEQ ID NO: 59
SYDMS
P1955 Kabat CDRH2 amino acid sequence
SEQ ID NO: 60
LIDGGGNTYYASWAKG
P1955 Kabat CDRH3 amino acid sequence
SEQ ID NO: 61
ELWPAYSSGAFNI
P1955 Kabat CDRL1 amino acid sequence
SEQ ID NO: 62
TADTLSRRYAS
P1955 Kabat CDRL2 amino acid sequence
SEQ ID NO: 63
RDTRRPS
P1955 Kabat CDRL3 amino acid sequence
SEQ ID NO: 64
VTTDGSGSSYLVV
amino acid sequence of mature heavy chain variable region of clone P2388
SEQ ID NO: 65
QEQLKESGGGLFKPTDTLTLTCTVSGFSLSNYAISLVRQPPGNGLEWIGAIDAGGVTY
YASWAKSRSTITRNTNLNTVTLKMTSLTAADTATYFCAREEWSSSSGLYFSIWGPGT
LVTISS
amino acid sequence of mature light chain variable region of clone P2388
SEQ ID NO: 66
SFVLTQPASVQVNLGQTVSLTCTADTLSRRYASWYQQKPGQAPVLLIYRDTSRPSGV
PDRFSGSSSGNTATLTISGAQAGDEADYYCATSDGSGSSYLYVFGGGTQLTVTG
P2388 Kabat CDRH1 amino acid sequence
SEQ ID NO: 67
NYAIS
P2388 Kabat CDRH2 amino acid sequence
SEQ ID NO: 68
AIDAGGVTYYASWAKS
P2388 Kabat CDRH3 amino acid sequence
SEQ ID NO: 69
EEWSSSSGLYFSI
P2388 Kabat CDRL1 amino acid sequence
SEQ ID NO: 70
TADTLSRRYAS
P2388 Kabat CDRL2 amino acid sequence
SEQ ID NO: 71
RDTSRPS
P2388 Kabat CDRL3 amino acid sequence
SEQ ID NO: 72
ATSDGSGSSYLYV
amino acid sequence of mature heavy chain variable region of clone P2653
SEQ ID NO: 73
QEQLEESGGRLVTPGTPLTLTCTASGFSLSTYAMTWVRQAPGKGLEWIGIIDRYGYT
YYANWARGRFTISKTSTTVDLKITSPTTEDTATYFCARDLPGYNVGTYFNMWGPGTL
VTISS
amino acid sequence of mature light chain variable region of clone P2653
SEQ ID NO: 74
SFVLTQPASVQVNLGQTVSLTCTADTLSRRYASWYQQKPGQAPVLFIYRDTSRPSGV
PDRFSGSSSGSTATLTISGAQAGDEADYYCATSDGSGSSYQFVFGGGTQLTVTG
P2653 Kabat CDRH1 amino acid sequence
SEQ ID NO: 75
TYAMT
P2653 Kabat CDRH2 amino acid sequence
SEQ ID NO: 76
IIDRYGYTYYANWARG
P2653 Kabat CDRH3 amino acid sequence
SEQ ID NO: 77
DLPGYNVGTYFNM
P2653 Kabat CDRL1 amino acid sequence
SEQ ID NO: 78
TADTLSRRYAS
P2653 Kabat CDRL2 amino acid sequence
SEQ ID NO: 79
RDTSRPS
P2653 Kabat CDRL3 amino acid sequence
SEQ ID NO: 80
ATSDGSGSSYQFV
amino acid sequence of mature heavy chain variable region of clone P2861
SEQ ID NO: 81
AQSVEESGGRLVTPGTPLTLTCTVSGIDLSSYAMGWVRQAPGKGLEYIGIIVSDGRTY
YTSWAKGRFTISKTSTTVDLKMTSPTTEDTATYFCAREGYGYYFDIWGPGTLVTISS
amino acid sequence of mature light chain variable region of clone P2861
SEQ ID NO: 82
SFVLTQPASVQVNLGQTVSLTCTADTLSRRYASWYQQKPGQAPVLLIYRDTSRPSGV
PDRFSGSSSGNTATLTISGAQAGDEADYYCATADGSGSSYQFVFGGGTQLTVTG
P2861 Kabat CDRH1 amino acid sequence
SEQ ID NO: 83
SYAMG
P2861 Kabat CDRH2 amino acid sequence
SEQ ID NO: 84
IIVSDGRTYYTSWAKG
P2861 Kabat CDRH3 amino acid sequence
SEQ ID NO: 85
EGYGYYFDI
P2861 Kabat CDRL1 amino acid sequence
SEQ ID NO: 86
TADTLSRRYAS
P2861 Kabat CDRL2 amino acid sequence
SEQ ID NO: 87
RDTSRPS
P2861 Kabat CDRL3 amino acid sequence
SEQ ID NO: 88
ATADGSGSSYQFV
amino acid sequence of mature heavy chain variable region of clone P3208
SEQ ID NO: 89
AQSVKESEGGLFKPTDTLTLTCTVSGFSLSSYAISWVRRAPGNGLEWIGAIAGYGDA
YYASWAKSRSTITRNTNENTVTLKMTSLTAADTATYFCARDDYNVDTGIWGPGTLV
TISS
amino acid sequence of mature light chain variable region of
clone P3208
SEQ ID NO: 90
SFVLTQPASVQVNLGQTVSLTCTADTLSRRYASWYQQKPGQAPVLLIYRDTSRPSGV
PDRFSGSSSGNTATLTISGAQAGDEADYYCATGDGSGSSYQYVFGGGTQLTVTG
P3208 Kabat CDRH1 amino acid sequence
SEQ ID NO: 91
SYAIS
P3208 Kabat CDRH2 amino acid sequence
SEQ ID NO: 92
AIAGYGDAYYASWAKS
P3208 Kabat CDRH3 amino acid sequence
SEQ ID NO: 93
DDYNVDTGI
P3208 Kabat CDRL1 amino acid sequence
SEQ ID NO: 94
TADTLSRRYAS
P3208 Kabat CDRL2 amino acid sequence
SEQ ID NO: 95
RDTSRPS
P3208 Kabat CDRL3 amino acid sequence
SEQ ID NO: 96
ATGDGSGSSYQYV
amino acid sequence of mature heavy chain variable region of clone N277
SEQ ID NO: 97
AQSLEESGGRLVTPGTPLTLTCTASGFTLSSYHMSWVRQAPGKGLEWIGIISSGGSIY
YASWAKGRFTISRTSTTVDLKMTSPTTEDTATYFCARYAAYSIGMDLWGPGTLVTVSS
amino acid sequence of mature light chain variable region of clone N277
SEQ ID NO: 98
QFVLTQPASVQVNLGQMVSLTCTADTLSRSYASWYQQKPGQAPVLLIYRDTSRPSG
VPDRFSGSTSGNTATLTISGAQAGDEADYYCATDDNSGSSYQYVFGGGTQLTVTG
N277 Kabat CDRH1 amino acid sequence
SEQ ID NO: 99
SYHMS
N277 Kabat CDRH2 amino acid sequence
SEQ ID NO: 100
IISSGGSIYYASWAKG
N277 Kabat CDRH3 amino acid sequence
SEQ ID NO: 101
YAAYSIGMDL
N277 Kabat CDRL1 amino acid sequence
SEQ ID NO: 102
TADTLSRSYAS
N277 Kabat CDRL2 amino acid sequence
SEQ ID NO: 103
RDTSRPS
N277 Kabat CDRL3 amino acid sequence
SEQ ID NO: 104
ATDDNSGSSYQYV
amino acid sequence of mature heavy chain variable region of clone N350
SEQ ID NO: 105
QEQLEESGGRLVTPGTPLTLTCTASGFSLSSYHMSWVRQAPGKGLEWIGLINTGDSA
YYASWAKGRFTISRTSTTVDLRITSPTTEDTATYFCARTFGDGFNLWGPGTLVTISS
amino acid sequence of mature light chain variable region of clone N350
SEQ ID NO: 106
SFVLTQPASVQVDLGQTVSLTCTADTLSRSYASWYQQKPGQAPVLLIYRDTSRPSGV
PDRFSGSSSGNTATLTISGAQAGDEADYYCATEDSGDSSYQYVFGGGTQLTVTG
N350 Kabat CDRH1 amino acid sequence
SEQ ID NO: 107
SYHMS
N350 Kabat CDRH2 amino acid sequence
SEQ ID NO: 108
LINTGDSAYYASWAKG
N350 Kabat CDRH3 amino acid sequence
SEQ ID NO: 109
TFGDGFNL
N350 Kabat CDRL1 amino acid sequence
SEQ ID NO: 110
TADTLSRSYAS
N350 Kabat CDRL2 amino acid sequence
SEQ ID NO: 111
RDTSRPS
N350 Kabat CDRL3 amino acid sequence
SEQ ID NO: 112
ATEDSGDSSYQYV
amino acid sequence of mature heavy chain variable region of clone N484
SEQ ID NO: 113
AQSLEESGGRLVTPGTPLTLTCTASGFSLSSYHMAWVRQAPGKGLEWIGIISSSGSTY
YASWAKGRFTISKTSTTVDLKITSPTTEDTATYFCARGFGWGPNLWGPGTLVTVSS
amino acid sequence of mature light chain variable region of clone N484
SEQ ID NO: 114
SFVLTQPASVQVNLGQMVSLTCTADTLSRSYASWYQQKPGQAPVLLIYRDTSRPSGV
PDRFSGSTSGNTATLTISGAQAGDEADYYCATDDNSGSSYQYVFGGGTQLTVTG
N484 Kabat CDRH1 amino acid sequence
SEQ ID NO: 115
SYHMA
N484 Kabat CDRH2 amino acid sequence
SEQ ID NO: 116
IISSSGSTYYASWAKG
N484 Kabat CDRH3 amino acid sequence
SEQ ID NO: 117
GFGWGPNL
N484 Kabat CDRL1 amino acid sequence
SEQ ID NO: 118
TADTLSRSYAS
N484 Kabat CDRL2 amino acid sequence
SEQ ID NO: 119
RDTSRPS
N484 Kabat CDRL3 amino acid sequence
SEQ ID NO: 120
ATDDNSGSSYQYV
amino acid sequence of mature heavy chain variable region of clone N2282
SEQ ID NO: 121
QEQLKESGGRLVTPGTPLTLTCTASGFTISSYHMSWVRQAPGKGLEWIGVISASGGST
YYASWAKGRFTISKTSTTVDLKITSPTTEDTATYFCARESYGGYNLWGPGTLVTVSS
amino acid sequence of mature light chain variable region of clone N2282
SEQ ID NO: 122
SFVLTQPASVQVNLGQMVSLTCTADTLSRSYASWYQQKPGQAPVLLIYRDTSRPSGV
PDRFSGSTSGNTATLTISGAQAGDEADYYCATDDNSGSSYQYVFGGGTQLTVTG
N2282 Kabat CDRH1 amino acid sequence
SEQ ID NO: 123
SYHMS
N2282 Kabat CDRH2 amino acid sequence
SEQ ID NO: 124
VISASGGSTYYASWAKG
N2282 Kabat CDRH3 amino acid sequence
SEQ ID NO: 125
ESYGGYNL
N2282 Kabat CDRL1 amino acid sequence
SEQ ID NO: 126
TADTLSRSYAS
N2282 Kabat CDRL2 amino acid sequence
SEQ ID NO: 127
RDTSRPS
N2282 Kabat CDRL3 amino acid sequence
SEQ ID NO: 128
ATDDNSGSSYQYV
amino acid sequence of mature heavy chain variable region of clone N2430
SEQ ID NO: 129
AQSLEESGGRLVTPGTPLTLTCTVSGIDLSSYHMNWVRQAPGKGLEWIGIIGASAGTY
YARWAKGRFTISKTSTTVDLKITSPTTEDTATYFCARSGGSMQLNLWGPGTLVTISS
amino acid sequence of mature light chain variable region of clone N2430
SEQ ID NO: 130
SFVLTQPASVQVNLGQTVSLTCTADTLSRSYASWYQQKPGQAPVLLIYRDTSRPSGV
PDRFSGSSSGNTATLTISGAQAGDEADYYCATDDSSGSSYQYVFGGGTQLTVTG
N2430 Kabat CDRH1 amino acid sequence
SEQ ID NO: 131
SYHMN
N2430 Kabat CDRH2 amino acid sequence
SEQ ID NO: 132
IIGASAGTYYARWAKG
N2430 Kabat CDRH3 amino acid sequence
SEQ ID NO: 133
SGGSMQLNL
N2430 Kabat CDRL1 amino acid sequence
SEQ ID NO: 134
TADTLSRSYAS
N2430 Kabat CDRL2 amino acid sequence
SEQ ID NO: 135
RDTSRPS
N2430 Kabat CDRL3 amino acid sequence
SEQ ID NO: 136
ATDDSSGSSYQYV
amino acid sequence of mature heavy chain variable region of clone E2810
SEQ ID NO: 137
QEQLEESGGRLVTPGTPLTLTCTASGFSLTNYYMTWVRQAPGKGLEWIGTIYAGTGS
TWYASWVKGRFTISKTSTTVDLKITSPTTEDTATYFCVRGYLGWSSGIENIWGPGTLV
TVSS
amino acid sequence of mature light chain variable region of clone E2810
SEQ ID NO: 138
SFVLTQPASVQVNLGQTVSLTCTADTLSRSYASWYQQKPGQAPVLLIYRDTSRPSGV
PDRFSGSSSGNTATLTISGAQAGDEADYYCATSDGSGSTFHFVFGGGTQLTVTG
E2810 Kabat CDRH1 amino acid sequence
SEQ ID NO: 139
NYYMT
E2810 Kabat CDRH2 amino acid sequence
SEQ ID NO: 140
TIYAGTGSTWYASWVKG
E2810 Kabat CDRH3 amino acid sequence
SEQ ID NO: 141
GYLGWSSGIENI
E2810 Kabat CDRL1 amino acid sequence
SEQ ID NO: 142
TADTLSRSYAS
E2810 Kabat CDRL2 amino acid sequence
SEQ ID NO: 143
RDTSRPS
E2810 Kabat CDRL3 amino acid sequence
SEQ ID NO: 144
ATSDGSGSTFHFV
amino acid sequence of Human PTPRG UniProt No. P23470
SEQ ID NO: 145
>sp|P23470|PTPRG HUMAN Receptor-type tyrosine-protein
phosphatase gamma OS = Homo sapiens OX = 9606 GN = PTPRG PE = 1 SV = 4
MRRLLEPCWWILFLKITSSVLHYVVCFPALTEGYVGALHENRHGSAVQIRRRKASGDPYWAY
SGAYGPEHWVTSSVSCGGRHQSPIDILDQYARVGEEYQELQLDGEDNESSNKTWMKNTGKTV
AILLKDDYFVSGAGLPGRFKAEKVEFHWGHSNGSAGSEHSINGRRFPVEMQIFFYNPDDEDS
FQTAISENRIIGAMAIFFQVSPRDNSALDPIIHGLKGVVHHEKETFLDPFVLRDLLPASLGS
YYRYTGSLTTPPCSEIVEWIVERRPVPISYHQLEAFYSIFTTEQQDHVKSVEYLRNNFRPQQ
RLHDRVVSKSAVRDSWNHDMTDFLENPLGTEASKVCSSPPIHMKVQPLNQTALQVSWSQPET
IYHPPIMNYMISYSWTKNEDEKEKTFTKDSDKDLKATISHVSPDSLYLFRVQAVCRNDMRSD
FSQTMLFQANTTRIFQGTRIVKTGVPTASPASSADMAPISSGSSTWTSSGIPFSFVSMATGM
GPSSSGSQATVASVVTSTLLAGLGFGGGGISSFPSTVWPTRLPTAASASKQAARPVLATTEA
LASPGPDGDSSPTKDGEGTEEGEKDEKSESEDGEREHEEDGEKDSEKKEKSGVTHAAEERNQ
TEPSPTPSSPNRTAEGGHQTIPGHEQDHTAVPTDQTGGRRDAGPGLDPDMVTSTQVPPTATE
EQYAGSDPKRPEMPSKKPMSRGDRFSEDSRFITVNPAEKNTSGMISRPAPGRMEWIIPLIVV
SALTFVCLILLIAVLVYWRGCNKIKSKGFPRRFREVPSSGERGEKGSRKCFQTAHFYVEDSS
SPRVVPNESIPIIPIPDDMEAIPVKQFVKHIGELYSNNQHGFSEDFEEVQRCTADMNITAEH
SNHPENKHKNRYINILAYDHSRVKLRPLPGKDSKHSDYINANYVDGYNKAKAYIATQGPLKS
TFEDFWRMIWEQNTGIIVMITNLVEKGRRKCDQYWPTENSEEYGNIIVTLKSTKIHACYTVR
RFSIRNTKVKKGQKGNPKGRQNERVVIQYHYTQWPDMGVPEYALPVLTFVRRSSAARMPETG
PVLVHCSAGVGRTGTYIVIDSMLQQIKDKSTVNVLGFLKHIRTQRNYLVQTEEQYIFIHDAL
LEAILGKETEVSSNQLHSYVNSILIPGVGGKTRLEKQFKLVTQCNAKYVECESAQKECNKEK
NRNSSVVPSERARVGLAPLPGMKGTDYINASYIMGYYRSNEFIITQHPLPHTTKDEWRMIWD
HNAQIIVMLPDNQSLAEDEFVYWPSREESMNCEAFTVTLISKDRLCLSNEEQIIIHDFILEA
TQDDYVLEVRHFQCPKWPNPDAPISSTFELINVIKEEALTRDGPTIVHDEYGAVSAGMLCAL
TTLSQQLENENAVDVFQVAKMINLMRPGVFTDIEQYQFIYKAMLSLVSTKENGNGPMTVDKN
GAVLIADESDPAESMESLV
amino acid sequence of Human Nectin-2 UniProt No. Q92692-1
SEQ ID NO: 146
MARAAALLPSRSPPTPLLWPLLLLLLLETGAQDVRVQVLPEVRGQLGGTVELPCHLL
PPVPGLYISLVTWQRPDAPANHQNVAAFHPKMGPSFPSPKPGSERLSFVSAKQSTGQ
DTEAELQDATLALHGLTVEDEGNYTCEFATFPKGSVRGMTWLRVIAKPKNQAEAQK
VTFSQDPTTVALCISKEGRPPARISWLSSLDWEAKETQVSGTLAGTVTVTSRFTLVPS
GRADGVTVTCKVEHESFEEPALIPVTLSVRYPPEVSISGYDDNWYLGRTDATLSCDV
RSNPEPTGYDWSTTSGTFPTSAVAQGSQLVIHAVDSLFNTTFVCTVTNAVGMGRAEQ
VIFVRETPNTAGAGATGGIIGGIIAAIIATAVAATGILICRQQRKEQTLQGAEEDEDLEG
PPSYKPPTPKAKLEAQEMPSQLFTLGASEHSPLKTPYFDAGASCTEQEMPRYHELPTL
EERSGPLHPGATSLGSPIPVPPGPPAVEDVSLDLEDEEGEEEEEYLDKINPIYDALSYSS
PSDSYQGKGFVMSRAMYV
amino acid sequence of Human endoglin UniProt No. P17813-1
SEQ ID NO: 147
MDRGTLPLAVALLLASCSLSPTSLAETVHCDLQPVGPERGEVTYTTSQVSKGCVAQA
PNAILEVHVLFLEFPTGPSQLELTLQASKQNGTWPREVLLVLSVNSSVFLHLQALGIPL
HLAYNSSLVTFQEPPGVNTTELPSFPKTQILEWAAERGPITSAAELNDPQSILLRLGQA
QGSLSFCMLEASQDMGRTLEWRPRTPALVRGCHLEGVAGHKEAHILRVLPGHSAGP
RTVTVKVELSCAPGDLDAVLILQGPPYVSWLIDANHNMQIWTTGEYSFKIFPEKNIRG
FKLPDTPQGLLGEARMLNASIVASFVELPLASIVSLHASSCGGRLQTSPAPIQTTPPKD
TCSPELLMSLIQTKCADDAMTLVLKKELVAHLKCTITGLTFWDPSCEAEDRGDKFVL
RSAYSSCGMQVSASMISNEAVVNILSSSSPQRKKVHCLNMDSLSFQLGLYLSPHFLQ
ASNTIEPGQQSFVQVRVSPSVSEFLLQLDSCHLDLGPEGGTVELIQGRAAKGNCVSLL
SPSPEGDPRFSFLLHFYTVPIPKTGTLSCTVALRPKTGSQDQEVHRTVFMRLNIISPDLS
GCTSKGLVLPAVLGITFGAFLIGALLTAALWYIYSHTRSPSKREPVVAVAAPASSESSS
TNHSIGSTQSTPCSTSSMA
amino acid sequence of UniProt No. Q05909 · PTPRG MOUSE
SEQ ID NO: 148
MRRLLEPCWWILFLKITSSVLHYVVCFPALTEGYVGTLQESRQDSSVQIRRRKASGDP
YWAYSGAYGPEHWVTSSVSCGGSHQSPIDILDHHARVGDEYQELQLDGFDNESSNK
TWMKNTGKTVAILLKDDYFVSGAGLPGRFKAEKVEFHWGHSNGSAGSEHSVNGRR
FPVEMQIFFYNPDDFDSFQTAISENRIIGAMAIFFQVSPRDNSALDPIIHGLKGVVHHE
KETFLDPFILRDLLPASLGSYYRYTGSLTTPPCSEIVEWIVFRRPVPISYHQLEAFYSIFT
TEQQDHVKSVEYLRNNFRPQQALNDRVVSKSAVRDAWNHDLADFLDNPLGTEASK
VCSSPPIHMKVQPLNQTALQVSWSQPETIYHPPIMNYMISYSWTKNEDEKEKTFTKD
SDKDLKATISHVSPDSLYLFRVQAVCRNDMRSDFSQTMLFQANTTRIFQGTRIVKTG
VPTASPASSADMAPISSGSSTWTSSGIPFSFVSMATGMGPSSSGSQATVASVVTSTLLA
GLGFGGGGISSFPSTVWPTRLPTASAASKQAGRTVLATTEALASPGPDVHSAPSKDSE
GTEEGEKEEKSESEDGEREHEEEEKDSEKKEKSEATHTAAESDRTAPAPTPSSPHRTA
AEGGHQTIPGRRQDHSAPATDQPGHVAPDLDPLVDTATQVPPTATEEHYSGSDPRRP
EMPSKKPMSRGDRFSEDSKFITVNPAEKNTSGMLSRPSPGRMEWIIPLIVVSALTFVCL
VLLIAVLVYWRGCNKIKSKGFPRRSREVPSSGERGEKGSRKCFQTAHFYVEDSSSPRV
VPNESVPIIPIPDDMEAIPVKQFGKHIGELYSNSQHGFSEDFEEVQRCTADMNITAEHS
NHPDNKHKNRYINILAYDHSRVKLRPLPGKDSKHSDYINANYVDGYNKAKAYIATQ
GPLKSTFEDFWRMIWEQNTGIIIMITNLVEKGRRKCDQYWPTENTEEYGNIIVTLKST
KVHACYTVRRLSVRNTKVKKGQKGNPKGRQNERTVIQYHYTQWPDMGVPEYALP
VLTFVRRSSAARMPDMGPVLVHCSAGVGRTGTYIVIDSMLQQIKDKSTVNVLGFLK
HIRTQRNYL VQTEEQYIFIHDALLEAILGKETAVSSSQLHSYVNSILIPGVGGKTRLEK
QFKLITQCNAKYVECFSAQKECNKEKNRNSSVVPAERARVGLAPLPGMKGTDYINA
SYIMGYYRSNEFIITQHPLPHTTKDFWRMIWDHNAQIIVMLPDNQSLAEDEFVYWPS
REESMNCEAFTVTLISKDRLCLSNEEQIIIHDFILEATQDDYVLEVRHFQCPKWPNPDA
PISSTFELINVIKEEALTRDGPTIVHDEYGAVSAGMLCALTTLSQQLENENAVDVFQV
AKMINLMRPGVFTDIEQYQFVYKAMLSLISTKENGNGPMTGDKNGAVLTAEESDPA
ESMESLV

We claim:

1. An isolated antibody that competes for binding to an acute myeloid leukemia (AML) cell surface antigen with antibody P11, P17, P28, P130, P240, P1187, P1707, P1955, P2388, P2653, P2861, P3208, N277, N350, N484, N2282, N2430, or E2810.

2. The antibody of claim 1 that binds to the same epitope on an AML cell surface antigen as antibody P11, P17, P28, P130, P240, P1187, P1707, P1955, P2388, P2653, P2861, P3208, N277, N350, N484, N2282, N2430, or E2810.

3. The antibody of claim 1 or claim 2, wherein the AML cell surface antigen is PTPRG, Nectin-1, or endoglin.

4. The antibody of any one of claims 1-3 specifically binding PTPRG
comprising three heavy chain CDRs and three light chain CDRs of antibody P11, wherein P11 is characterized by a mature heavy chain variable region having an amino acid sequence comprising SEQ ID NO:1 and a mature light chain variable region having an amino acid sequence comprising SEQ ID NO:2;
comprising three heavy chain CDRs and three light chain CDRs of antibody P17, wherein P17 is characterized by a mature heavy chain variable region having an amino acid sequence comprising SEQ ID NO:9 and a mature light chain variable region having an amino acid sequence comprising SEQ ID NO:10;
comprising three heavy chain CDRs and three light chain CDRs of antibody P28, wherein P28 is characterized by a mature heavy chain variable region having an amino acid sequence comprising SEQ ID NO:17 and a mature light chain variable region having an amino acid sequence comprising SEQ ID NO: 18;
comprising three heavy chain CDRs and three light chain CDRs of antibody P130, wherein P130 is characterized by a mature heavy chain variable region having an amino acid sequence comprising SEQ ID NO:25 and a mature light chain variable region having an amino acid sequence comprising SEQ ID NO:26;
comprising three heavy chain CDRs and three light chain CDRs of antibody P240, wherein P240 is characterized by a mature heavy chain variable region having an amino acid sequence comprising SEQ ID NO:33 and a mature light chain variable region having an amino acid sequence comprising SEQ ID NO:34;
comprising three heavy chain CDRs and three light chain CDRs of antibody P1187, wherein P1187 is characterized by a mature heavy chain variable region having an amino acid sequence comprising SEQ ID NO:41 and a mature light chain variable region having an amino acid sequence comprising SEQ ID NO:42;
comprising three heavy chain CDRs and three light chain CDRs of antibody P1707, wherein P1707 is characterized by a mature heavy chain variable region having an amino acid sequence comprising SEQ ID NO:49 and a mature light chain variable region having an amino acid sequence comprising SEQ ID NO:50;
comprising three heavy chain CDRs and three light chain CDRs of antibody P1955, wherein P1955 is characterized by a mature heavy chain variable region having an amino acid sequence comprising SEQ ID NO:57 and a mature light chain variable region having an amino acid sequence comprising SEQ ID NO:58;
comprising three heavy chain CDRs and three light chain CDRs of antibody P2388, wherein P2388 is characterized by a mature heavy chain variable region having an amino acid sequence comprising SEQ ID NO:65 and a mature light chain variable region having an amino acid sequence comprising SEQ ID NO:66;
comprising three heavy chain CDRs and three light chain CDRs of antibody P2653, wherein P2653 is characterized by a mature heavy chain variable region having an amino acid sequence comprising SEQ ID NO:73 and a mature light chain variable region having an amino acid sequence comprising SEQ ID NO:74;
comprising three heavy chain CDRs and three light chain CDRs of antibody P2861, wherein P2861 is characterized by a mature heavy chain variable region having an amino acid sequence comprising SEQ ID NO:81 and a mature light chain variable region having an amino acid sequence comprising SEQ ID NO:82; or
comprising three heavy chain CDRs and three light chain CDRs of antibody P3208, wherein P3208 is characterized by a mature heavy chain variable region having an amino acid sequence comprising SEQ ID NO:89 and a mature light chain variable region having an amino acid sequence comprising SEQ ID NO:90.

5. The antibody of claim 4, wherein the CDRs are of a definition selected from the group consisting of Kabat, Chothia, Kabat/Chothia Composite, AbM, Contact, and IMGT.

6. The antibody of claim 5,
wherein the mature heavy chain variable region comprises the three Kabat heavy chain CDRs of SEQ ID NOs:3-5 and the mature light chain variable region comprises the three Kabat light chain CDRs of SEQ ID NOs:6-8;
wherein the mature heavy chain variable region comprises the three Kabat heavy chain CDRs of SEQ ID NOs:11-13 and the mature light chain variable region comprises the three Kabat light chain CDRs of SEQ ID NOs:14-16;
wherein the mature heavy chain variable region comprises the three Kabat heavy chain CDRs of SEQ ID NOs:19-21 and the mature light chain variable region comprises the three Kabat light chain CDRs of SEQ ID NOs:22-24;
wherein the mature heavy chain variable region comprises the three Kabat heavy chain CDRs of SEQ ID NOs:27-29 and the mature light chain variable region comprises the three Kabat light chain CDRs of SEQ ID NOs:30-32;
wherein the mature heavy chain variable region comprises the three Kabat heavy chain CDRs of SEQ ID NOs:35-37 and the mature light chain variable region comprises the three Kabat light chain CDRs of SEQ ID NOs:38-40;
wherein the mature heavy chain variable region comprises the three Kabat heavy chain CDRs of SEQ ID NOs:43-45 and the mature light chain variable region comprises the three Kabat light chain CDRs of SEQ ID NOs:46-48;
wherein the mature heavy chain variable region comprises the three Kabat heavy chain CDRs of SEQ ID NOs:51-53 and the mature light chain variable region comprises the three Kabat light chain CDRs of SEQ ID NOs:54-56;
wherein the mature heavy chain variable region comprises the three Kabat heavy chain CDRs of SEQ ID NOs:59-61 and the mature light chain variable region comprises the three Kabat light chain CDRs of SEQ ID NOs:62-64;
wherein the mature heavy chain variable region comprises the three Kabat heavy chain CDRs of SEQ ID NOs:67-69 and the mature light chain variable region comprises the three Kabat light chain CDRs of SEQ ID NOs:70-72;
wherein the mature heavy chain variable region comprises the three Kabat heavy chain CDRs of SEQ ID NOs:75-77 and the mature light chain variable region comprises the three Kabat light chain CDRs of SEQ ID NOs:78-80;
wherein the mature heavy chain variable region comprises the three Kabat heavy chain CDRs of SEQ ID NOs:83-85 and the mature light chain variable region comprises the three Kabat light chain CDRs of SEQ ID NOs:86-88; or
wherein the mature heavy chain variable region comprises the three Kabat heavy chain CDRs of SEQ ID NOs:91-93 and the mature light chain variable region comprises the three Kabat light chain CDRs of SEQ ID NOs:94-96;

7. The antibody of any one of claims 1-6,
comprising a mature heavy chain variable region having an amino acid sequence at least 90% identical to SEQ ID NO:1, and a mature light chain variable region having an amino acid sequence at least 90% identical to SEQ ID NO:2;
comprising a mature heavy chain variable region having an amino acid sequence at least 90% identical to SEQ ID NO:9, and a mature light chain variable region having an amino acid sequence at least 90% identical to SEQ ID NO:10;
comprising a mature heavy chain variable region having an amino acid sequence at least 90% identical to SEQ ID NO:17, and a mature light chain variable region having an amino acid sequence at least 90% identical to SEQ ID NO:18;
comprising a mature heavy chain variable region having an amino acid sequence at least 90% identical to SEQ ID NO:25, and a mature light chain variable region having an amino acid sequence at least 90% identical to SEQ ID NO:26;
comprising a mature heavy chain variable region having an amino acid sequence at least 90% identical to SEQ ID NO:33, and a mature light chain variable region having an amino acid sequence at least 90% identical to SEQ ID NO:34;
comprising a mature heavy chain variable region having an amino acid sequence at least 90% identical to SEQ ID NO:41, and a mature light chain variable region having an amino acid sequence at least 90% identical to SEQ ID NO:42;
comprising a mature heavy chain variable region having an amino acid sequence at least 90% identical to SEQ ID NO:49, and a mature light chain variable region having an amino acid sequence at least 90% identical to SEQ ID NO:50;
comprising a mature heavy chain variable region having an amino acid sequence at least 90% identical to SEQ ID NO:57, and a mature light chain variable region having an amino acid sequence at least 90% identical to SEQ ID NO:58;
comprising a mature heavy chain variable region having an amino acid sequence at least 90% identical to SEQ ID NO:65, and a mature light chain variable region having an amino acid sequence at least 90% identical to SEQ ID NO:66;
comprising a mature heavy chain variable region having an amino acid sequence at least 90% identical to SEQ ID NO:73, and a mature light chain variable region having an amino acid sequence at least 90% identical to SEQ ID NO:74;
comprising a mature heavy chain variable region having an amino acid sequence at least 90% identical to SEQ ID NO:81, and a mature light chain variable region having an amino acid sequence at least 90% identical to SEQ ID NO:82; or
comprising a mature heavy chain variable region having an amino acid sequence at least 90% identical to SEQ ID NO:89, and a mature light chain variable region having an amino acid sequence at least 90% identical to SEQ ID NO:90.

8. The antibody of claim 7,
wherein the mature heavy chain variable region has an amino acid sequence of SEQ ID NO:1 and the mature light chain variable region has an amino acid sequence of SEQ ID NO: 2;
wherein the mature heavy chain variable region has an amino acid sequence of SEQ ID NO:9 and the mature light chain variable region has an amino acid sequence of SEQ ID NO: 10;
wherein the mature heavy chain variable region has an amino acid sequence of SEQ ID NO: 17 and the mature light chain variable region has an amino acid sequence of SEQ ID NO:18;
wherein the mature heavy chain variable region has an amino acid sequence of SEQ ID NO:25 and the mature light chain variable region has an amino acid sequence of SEQ ID NO:26;
wherein the mature heavy chain variable region has an amino acid sequence of SEQ ID NO:33 and the mature light chain variable region has an amino acid sequence of SEQ ID NO:34;
wherein the mature heavy chain variable region has an amino acid sequence of SEQ ID NO:41 and the mature light chain variable region has an amino acid sequence of SEQ ID NO:42;
wherein the mature heavy chain variable region has an amino acid sequence of SEQ ID NO:49 and the mature light chain variable region has an amino acid sequence of SEQ ID NO:50;
wherein the mature heavy chain variable region has an amino acid sequence of SEQ ID NO:57 and the mature light chain variable region has an amino acid sequence of SEQ ID NO:58;
wherein the mature heavy chain variable region has an amino acid sequence of SEQ ID NO:65 and the mature light chain variable region has an amino acid sequence of SEQ ID NO:66;
wherein the mature heavy chain variable region has an amino acid sequence of SEQ ID NO:73 and the mature light chain variable region has an amino acid sequence of SEQ ID NO:74;
wherein the mature heavy chain variable region has an amino acid sequence of SEQ ID NO:81 and the mature light chain variable region has an amino acid sequence of SEQ ID NO:82; or
wherein the mature heavy chain variable region has an amino acid sequence of SEQ ID NO:89 and the mature light chain variable region has an amino acid sequence of SEQ ID NO:90.

9. The antibody of any one of claims 1-3 specifically binding Nectin-2
comprising three heavy chain CDRs and three light chain CDRs of antibody N277, wherein N277 is characterized by a mature heavy chain variable region having an amino acid sequence comprising SEQ ID NO:97 and a mature light chain variable region having an amino acid sequence comprising SEQ ID NO:98;
comprising three heavy chain CDRs and three light chain CDRs of antibody N350, wherein N350 is characterized by a mature heavy chain variable region having an amino acid sequence comprising SEQ ID NO: 105 and a mature light chain variable region having an amino acid sequence comprising SEQ ID NO:106;
comprising three heavy chain CDRs and three light chain CDRs of antibody N484, wherein N484 is characterized by a mature heavy chain variable region having an amino acid sequence comprising SEQ ID NO: 113 and a mature light chain variable region having an amino acid sequence comprising SEQ ID NO 114;
comprising three heavy chain CDRs and three light chain CDRs of antibody N2282, wherein N2282 is characterized by a mature heavy chain variable region having an amino acid sequence comprising SEQ ID NO: 121 and a mature light chain variable region having an amino acid sequence comprising SEQ ID NO: 122; or
comprising three heavy chain CDRs and three light chain CDRs of antibody N2430, wherein N2430 is characterized by a mature heavy chain variable region having an amino acid sequence comprising SEQ ID NO: 129 and a mature light chain variable region having an amino acid sequence comprising SEQ ID NO:130.

10. The antibody of claim 9, wherein the CDRs are of a definition selected from the group consisting of Kabat, Chothia, Kabat/Chothia Composite, AbM, Contact, and IMGT.

11. The antibody of claim 10,
wherein the mature heavy chain variable region comprises the three Kabat heavy chain CDRs of SEQ ID NOs:99-101 and the mature light chain variable region comprises the three Kabat light chain CDRs of SEQ ID NOs:102-104;
wherein the mature heavy chain variable region comprises the three Kabat heavy chain CDRs of SEQ ID NOs:107-109 and the mature light chain variable region comprises the three Kabat light chain CDRs of SEQ ID NOs:110-112;
wherein the mature heavy chain variable region comprises the three Kabat heavy chain CDRs of SEQ ID NOs:115-117 and the mature light chain variable region comprises the three Kabat light chain CDRs of SEQ ID NOs:118-120;
wherein the mature heavy chain variable region comprises the three Kabat heavy chain CDRs of SEQ ID NOs:123-125 and the mature light chain variable region comprises the three Kabat light chain CDRs of SEQ ID NOs:126-128; or
wherein the mature heavy chain variable region comprises the three Kabat heavy chain CDRs of SEQ ID NOs:131-133 and the mature light chain variable region comprises the three Kabat light chain CDRs of SEQ ID NOs:134-136.

12. The antibody of any one of claims 1-3 and 9-11,
comprising a mature heavy chain variable region having an amino acid sequence at least 90% identical to SEQ ID NO:97, and a mature light chain variable region having an amino acid sequence at least 90% identical to SEQ ID NO:98;
comprising a mature heavy chain variable region having an amino acid sequence at least 90% identical to SEQ ID NO:105, and a mature light chain variable region having an amino acid sequence at least 90% identical to SEQ ID NO:106;
comprising a mature heavy chain variable region having an amino acid sequence at least 90% identical to SEQ ID NO:113, and a mature light chain variable region having an amino acid sequence at least 90% identical to SEQ ID NO:114;
comprising a mature heavy chain variable region having an amino acid sequence at least 90% identical to SEQ ID NO:121, and a mature light chain variable region having an amino acid sequence at least 90% identical to SEQ ID NO:122; or
comprising a mature heavy chain variable region having an amino acid sequence at least 90% identical to SEQ ID NO:129, and a mature light chain variable region having an amino acid sequence at least 90% identical to SEQ ID NO:130.

13. The antibody of claim 12,
wherein the mature heavy chain variable region has an amino acid sequence of SEQ ID NO:97 and the mature light chain variable region has an amino acid sequence of SEQ ID NO:98;
wherein the mature heavy chain variable region has an amino acid sequence of SEQ ID NO: 105 and the mature light chain variable region has an amino acid sequence of SEQ ID NO: 106;
wherein the mature heavy chain variable region has an amino acid sequence of SEQ ID NO: 113 and the mature light chain variable region has an amino acid sequence of SEQ ID NO:114;
wherein the mature heavy chain variable region has an amino acid sequence of SEQ ID NO: 121 and the mature light chain variable region has an amino acid sequence of SEQ ID NO:122; or
wherein the mature heavy chain variable region has an amino acid sequence of SEQ ID NO: 129 and the mature light chain variable region has an amino acid sequence of SEQ ID NO:130.

14. The antibody of any one of claims 1-3 specifically binding endoglin
comprising three heavy chain CDRs and three light chain CDRs of antibody E2810, wherein E2810 is characterized by a mature heavy chain variable region having an amino acid sequence comprising SEQ ID NO:137 and a mature light chain variable region having an amino acid sequence comprising SEQ ID NO:138.

15. The antibody of claim 14, wherein the CDRs are of a definition selected from the group consisting of Kabat, Chothia, Kabat/Chothia Composite, AbM, Contact, and IMGT.

16. The antibody of claim 15,
wherein the mature heavy chain variable region comprises the three Kabat heavy chain CDRs of SEQ ID NOs:139-141 and the mature light chain variable region comprises the three Kabat light chain CDRs of SEQ ID NOs:142-144.

17. The antibody of any one of claims 1-3 and 14-16,
comprising a mature heavy chain variable region having an amino acid sequence at least 90% identical to SEQ ID NO:137, and a mature light chain variable region having an amino acid sequence at least 90% identical to SEQ ID NO:138.

18. The antibody of claim 17,
wherein the mature heavy chain variable region has an amino acid sequence of SEQ ID NO: 137 and the mature light chain variable region has an amino acid sequence of SEQ ID NO:138.

19. The antibody of any preceding claim, wherein the antibody is chimeric or humanized.

20. The antibody of any preceding claim, wherein the antibody is IgAQ1, IgA2, IgD, IgE, IgG1, IgG2, IgG3, IgG4, synthetic IgG, IgM, F(ab)2, Fv, scFv, IgGACH2, F(ab′)2, scFv2CH3, Fab, VL, VH, scFv4, scFv3, scFv2, dsFv, Fv, scFv-Fc, (scFv)2, a non-depleting IgG, a diabody, or a bivalent antibody.

21. The antibody of any preceding claim wherein the mature light chain variable region is fused to a light chain constant region and the mature heavy chain variable region is fused to a heavy chain constant region.

22. The antibody of claim 21, wherein the heavy chain constant region is a mutant form of a natural human heavy chain constant region which has reduced binding to a Fcγ receptor relative to the natural human heavy chain constant region.

23. The antibody of any preceding claim, which an antibody fragment.

24. The antibody of any one of claims 1-22, which is a bispecific antibody.

25. The antibody of claim 24, wherein the bispecific antibody is a T-cell engaging bispecific antibody.

26. The antibody of claim 24, wherein the bispecific antibody is an NK-cell engaging bispecific antibody.

27. The antibody of any one of claims 1-22, which is an antibody-based binding protein.

28. The antibody of any preceding claim, which is conjugated to a synthetic molecule.

29. The antibody of claim 28, wherein the synthetic molecule is a cytotoxic agent, a label, a therapeutic radioisotope, or a liposome.

30. The antibody of claim 29, wherein the cytotoxic agent is a small molecule weight toxin, a peptide toxin, or a protein toxin.

31. The antibody of any one of claims 1-27 linked to at least one cytotoxic agent as an antibody drug conjugate.

32. The antibody of claim 31, wherein the cytotoxic agent is a small molecular weight toxin, a peptide toxin, or a protein toxin.

33. The antibody of claim 31, wherein the antibody is chimeric or humanized.

34. A chimeric antigen receptor (CAR), comprising the antibody of any one of claims 1-27 that is fused to a transmembrane region and an intracellular T-cell receptor (TCR) signaling domain, an intracellular T-cell costimulatory receptor signaling domain, or a signaling domain of a protein downstream of TCR or T-cell costimulatory receptor.

35. The chimeric antigen receptor of claim 34, wherein the antibody is chimeric or humanized.

36. A pharmaceutical composition comprising a therapeutically effective amount of the antibody of any one of claims 1-33 and a pharmaceutically acceptable carrier.

37. A kit comprising the antibody of any one of claims 1-33.

38. A nucleic acid encoding the heavy chain and/or light chain of an antibody as described in any one of claims 1-33.

39. A recombinant expression vector comprising a nucleic acid of claim 38.

40. A method of killing or inhibiting the growth of a cell expressing an acute myeloid leukemia cell surface antigen in a subject, comprising administering a pharmaceutical composition of claim 36 to a subject in need thereof, thereby killing or inhibiting the growth of the cell expressing the acute myeloid leukemia cell surface antigen in the subject.

41. The method of claim 40, wherein the cell is a tumor cell.

42. The method of claim 40, wherein the pharmaceutical composition comprises an antibody drug conjugate (ADC) comprising the antibody of any one of claims 1-27 and a cytotoxic agent.

43. The method of claim 42, wherein the cytotoxic agent is a small molecular weight toxin, a peptide toxin, or a protein toxin.

44. A method of treating a hematologic malignancy in a subject, comprising administering a pharmaceutical composition of claim 36 to the subject having the hematologic malignancy, thereby treating the hematologic malignancy in the subject.

45. The method of claim 44, wherein the hematologic malignancy is acute myeloid leukemia.

46. The method of claim 44, wherein the pharmaceutical composition comprises an antibody drug conjugate (ADC) comprising the antibody of any one of claims 1-27 and a cytotoxic agent.

47. The method of claim 46, wherein the cytotoxic agent is a small molecular weight toxin, a peptide toxin, or a protein toxin.

48. The method of claim 44, wherein the antibody is fused to a transmembrane region and an intracellular T-cell receptor (TCR) signaling domain, an intracellular T-cell costimulatory receptor signaling domain, or a signaling domain of a protein downstream of TCR or T-cell costimulatory receptor to form a chimeric antigen receptor (CAR).


SEQUENCE LISTING (148 sequences)

SEQ ID NO: 1 - protein, 116 aa, Synthetic construct
AQSLEESGGRLVTPGTPLTLTCTVSGFSLSSYAMTWVRQAPGEGLEWIGTIGAGGSPYYASWAKGRFTISKTATTVNLKIISPTTEDTATYFCAREGGGSYYFDLWGPGTLVTVSS

SEQ ID NO: 2 - protein, 111 aa, Synthetic construct
SFVLTQPASVQVNLGQTVSLTCTADTLSRSYASWYQQKPGQAPVLLIYRDTSRPSGVPDRFSGSSSGNTATLTISGAQAGDEADYYCATSDGSGSTFHFVFGGGTQLTVTG

SEQ ID NO: 3 - protein, 5 aa, Synthetic construct
SYAMT

SEQ ID NO: 4 - protein, 16 aa, Synthetic construct
TIGAGGSPYYASWAKG

SEQ ID NO: 5 - protein, 10 aa, Synthetic construct
EGGGSYYFDL

SEQ ID NO: 6 - protein, 11 aa, Synthetic construct
TADTLSRSYAS

SEQ ID NO: 7 - protein, 7 aa, Synthetic construct
RDTSRPS

SEQ ID NO: 8 - protein, 13 aa, Synthetic construct
ATSDGSGSTFHFV

SEQ ID NO: 9 - protein, 115 aa, Synthetic construct
AQSLEEFGGRLATPGTPLTLTCTASGFSLGTYSMSWVRQAPGKGLEWIGTIGGRGDAYYASWAKGRFTISRTSTTVDLKMTSLATEDTATYFCVRENYGYAFDIWGPGTLVTISS

SEQ ID NO: 10 - protein, 111 aa, Synthetic construct
SFVLTQPASVQVNLGQTVSLTCTADTLSRRYASWYQQKPGQAPVLLIYRDTSRPSGVPDRFSGSSSGNTATLTISGAQAGDEADYYCATSDGSGSSYQCVFGGGTQLTVTG

SEQ ID NO: 11 - protein, 5 aa, Synthetic construct
TYSMS

SEQ ID NO: 12 - protein, 16 aa, Synthetic construct
TIGGRGDAYYASWAKG

SEQ ID NO: 13 - protein, 9 aa, Synthetic construct
ENYGYAFDI

SEQ ID NO: 14 - protein, 11 aa, Synthetic construct
TADTLSRRYAS

SEQ ID NO: 15 - protein, 7 aa, Synthetic construct
RDTSRPS

SEQ ID NO: 16 - protein, 13 aa, Synthetic construct
ATSDGSGSSYQCV

SEQ ID NO: 17 - protein, 117 aa, Synthetic construct
AQSVEESGGRLVTPGIPLTLTCTVSGFSLNNYAMSWVRQAPGKGLEWIGMIDSGGAAYYATWARGRFTISRTSTTVDLKITSPTTEDTATYFCARELSSLYGAFDPWGPGTLVTVSS

SEQ ID NO: 18 - protein, 111 aa, Synthetic construct; identical to SEQ ID NO: 10

SEQ ID NO: 19 - protein, 5 aa, Synthetic construct
NYAMS

SEQ ID NO: 20 - protein, 16 aa, Synthetic construct
MIDSGGAAYYATWARG

SEQ ID NO: 21 - protein, 11 aa, Synthetic construct
ELSSLYGAFDP

SEQ ID NO: 22 - protein, 11 aa, Synthetic construct
TADTLSRRYAS

SEQ ID NO: 23 - protein, 7 aa, Synthetic construct
RDTSRPS

SEQ ID NO: 24 - protein, 13 aa, Synthetic construct
ATSDGSGSSYQCV

SEQ ID NO: 25 - protein, 120 aa, Synthetic construct
QEQQKESGGGLFKPTDTLTLTCTVSGFSLSSNAISWVRQAPGNGLEWIGAIDNSGSAYYATWAKGRSTITRATNLNTVTLKMTSVTAADTATYFCAREDVRADSIDLNMWGPGTLVTISS

SEQ ID NO: 26 - protein, 111 aa, Synthetic construct
SFVLTQPASVQVNLGQTVSLTCTADTLSRSYASWYQQKPGQAPVLLIYRDTSRPSGVPDRFSGSSSGNTATLTISGAQAGDEADYYCATSDGSGSSYQFVFGGGTQLTVTG

SEQ ID NO: 27 - protein, 5 aa, Synthetic construct
SNAIS

SEQ ID NO: 28 - protein, 16 aa, Synthetic construct
AIDNSGSAYYATWAKG

SEQ ID NO: 29 - protein, 12 aa, Synthetic construct
EDVRADSIDLNM

SEQ ID NO: 30 - protein, 11 aa, Synthetic construct
TADTLSRSYAS

SEQ ID NO: 31 - protein, 7 aa, Synthetic construct
RDTSRPS

SEQ ID NO: 32 - protein, 13 aa, Synthetic construct
ATSDGSGSSYQFV

SEQ ID NO: 33 - protein, 115 aa, Synthetic construct
AQSLEESGGRLVTPGTPLTLTCTVSGFSLSAYAMSWVRQAPGKGLEWIGIISSYGNTYYATWAKGRFTISKTSTTVDLKITSPTTEDTATYFCARGDYGNGMSIWGPGTLVTVSS

SEQ ID NO: 34 - protein, 111 aa, Synthetic construct
SFVLTQPASVQVNLGQTVSLTCTADTLSRRYASWYQQKPGQAPVLLIYRDTRRPSGVPDRFSGSSSGNTATLTISGAQAGDEADYYCATSDGSGSSYQCVFGGGTQLTVTG

SEQ ID NO: 35 - protein, 5 aa, Synthetic construct
AYAMS

SEQ ID NO: 36 - protein, 16 aa, Synthetic construct
IISSYGNTYYATWAKG

SEQ ID NO: 37 - protein, 9 aa, Synthetic construct
GDYGNGMSI

SEQ ID NO: 38 - protein, 11 aa, Synthetic construct
TADTLSRRYAS

SEQ ID NO: 39 - protein, 7 aa, Synthetic construct
RDTRRPS

SEQ ID NO: 40 - protein, 13 aa, Synthetic construct
ATSDGSGSSYQCV

SEQ ID NO: 41 - protein, 117 aa, Synthetic construct
AQSVEESGGRLVTPGTPLTLTCTVSGIDLSSYAMSWVRQAPGKGLEWIGVIDGSGSTWYANWVKGRFTISKTTTVDLKITSPTTEDTATYFCAREDYGSETIDFNIWGPGTLVTVSS

SEQ ID NO: 42 - protein, 111 aa, Synthetic construct
SFVLTQPASVQVNLGQTVSLTCTADTLSRRYASWYQQKPGQAPVLLIYRDTRRPSGVPDRFSGSSSGNTATLTISGAQAGDEADYYCATSDGSGSSYQYVFGGGTQLTVTG

SEQ ID NO: 43 - protein, 5 aa, Synthetic construct
SYAMS

SEQ ID NO: 44 - protein, 16 aa, Synthetic construct
VIDGSGSTWYANWVKG

SEQ ID NO: 45 - protein, 12 aa, Synthetic construct
EDYGSETIDFNI

SEQ ID NO: 46 - protein, 11 aa, Synthetic construct
TADTLSRRYAS

SEQ ID NO: 47 - protein, 7 aa, Synthetic construct
RDTRRPS

SEQ ID NO: 48 - protein, 13 aa, Synthetic construct
ATSDGSGSSYQYV

SEQ ID NO: 49 - protein, 121 aa, Synthetic construct
AQSLEEFGGGLIKPGGTLTLTCTASGFTISSYDMSWVRQAPGSGLEWIGTMAYGGDTYHASWAKSRSTITRDTNLNTVTLKMTSLTVADTATYFCARDVGSGHYYDDMYLWGPGTLVTVSS

SEQ ID NO: 50 - protein, 111 aa, Synthetic construct
SFVLTQPASVQVNFGQTVSLTCTADTLSRRYASWYQQKPGQAPVLLIYRDTSRPSGVPDRFSGSSSGNTATLTISGAQAGDEADYYCATGDGSGSSYNYVFGGGTQLTVTG

SEQ ID NO: 51 - protein, 5 aa, Synthetic construct
SYDMS

SEQ ID NO: 52 - protein, 16 aa, Synthetic construct
TMAYGGDTYHASWAKS

SEQ ID NO: 53 - protein, 13 aa, Synthetic construct
DVGSGHYYDDMYL

SEQ ID NO: 54 - protein, 11 aa, Synthetic construct
TADTLSRRYAS

SEQ ID NO: 55 - protein, 7 aa, Synthetic construct
RDTSRPS

SEQ ID NO: 56 - protein, 13 aa, Synthetic construct
ATGDGSGSSYNYV

SEQ ID NO: 57 - protein, 120 aa, Synthetic construct
AQSLEESGGRLVTPGTPLTLTCTASGFSLYSYDMSWVRQAPGKGLEWIGLIDGGGNTYYASWAKGRFTISKTSSTTVDLKMTSLTTEDTATYFCARELWPAYSSGAFNIWGPGTLVTISS

SEQ ID NO: 58 - protein, 111 aa, Synthetic construct
SFVLTQPASVQVNLGQTVSLTCTADTLSRRYASWYQQKPGQAPVLLIYRDTRRPSGVPDRFSGSSSGNTATLTISGAQAGDEADYYCVTTDGSGSSYLVVFGGGTQLTVTG

SEQ ID NO: 59 - protein, 5 aa, Synthetic construct
SYDMS

SEQ ID NO: 60 - protein, 16 aa, Synthetic construct
LIDGGGNTYYASWAKG

SEQ ID NO: 61 - protein, 13 aa, Synthetic construct
ELWPAYSSGAFNI

SEQ ID NO: 62 - protein, 11 aa, Synthetic construct
TADTLSRRYAS

SEQ ID NO: 63 - protein, 7 aa, Synthetic construct
RDTRRPS

SEQ ID NO: 64 - protein, 13 aa, Synthetic construct
VTTDGSGSSYLVV

SEQ ID NO: 65 - protein, 121 aa, Synthetic construct
QEQLKESGGGLFKPTDTLTLTCTVSGFSLSNYAISLVRQPPGNGLEWIGAIDAGGVTYYASWAKSRSTITRNTNLNTVTLKMTSLTAADTATYFCAREEWSSSSGLYFSIWGPGTLVTISS

SEQ ID NO: 66 - protein, 111 aa, Synthetic construct
SFVLTQPASVQVNLGQTVSLTCTADTLSRRYASWYQQKPGQAPVLLIYRDTSRPSGVPDRFSGSSSGNTATLTISGAQAGDEADYYCATSDGSGSSYLYVFGGGTQLTVTG

SEQ ID NO: 67 - protein, 5 aa, Synthetic construct
NYAIS

SEQ ID NO: 68 - protein, 16 aa, Synthetic construct
AIDAGGVTYYASWAKS

SEQ ID NO: 69 - protein, 13 aa, Synthetic construct
EEWSSSSGLYFSI

SEQ ID NO: 70 - protein, 11 aa, Synthetic construct
TADTLSRRYAS

SEQ ID NO: 71 - protein, 7 aa, Synthetic construct
RDTSRPS

SEQ ID NO: 72 - protein, 13 aa, Synthetic construct
ATSDGSGSSYLYV

SEQ ID NO: 73 - protein, 119 aa, Synthetic construct
QEQLEESGGRLVTPGTPLTLTCTASGFSLSTYAMTWVRQAPGKGLEWIGIIDRYGYTYYANWARGRFTISKTSTTVDLKITSPTTEDTATYFCARDLPGYNVGTYFNMWGPGTLVTISS

SEQ ID NO: 74 - protein, 111 aa, Synthetic construct
SFVLTQPASVQVNLGQTVSLTCTADTLSRRYASWYQQKPGQAPVLFIYRDTSRPSGVPDRFSGSSSGSTATLTISGAQAGDEADYYCATSDGSGSSYQFVFGGGTQLTVTG

SEQ ID NO: 75 - protein, 5 aa, Synthetic construct
TYAMT

SEQ ID NO: 76 - protein, 16 aa, Synthetic construct
IIDRYGYTYYANWARG

SEQ ID NO: 77 - protein, 13 aa, Synthetic construct
DLPGYNVGTYFNM

SEQ ID NO: 78 - protein, 11 aa, Synthetic construct
TADTLSRRYAS

SEQ ID NO: 79 - protein, 7 aa, Synthetic construct
RDTSRPS

SEQ ID NO: 80 - protein, 13 aa, Synthetic construct
ATSDGSGSSYQFV

SEQ ID NO: 81 - protein, 115 aa, Synthetic construct
AQSVEESGGRLVTPGTPLTLTCTVSGIDLSSYAMGWVRQAPGKGLEYIGIIVSDGRTYYTSWAKGRFTISKTSTTVDLKMTSPTTEDTATYFCAREGYGYYFDIWGPGTLVTISS

SEQ ID NO: 82 - protein, 111 aa, Synthetic construct
SFVLTQPASVQVNLGQTVSLTCTADTLSRRYASWYQQKPGQAPVLLIYRDTSRPSGVPDRFSGSSSGNTATLTISGAQAGDEADYYCATADGSGSSYQFVFGGGTQLTVTG

SEQ ID NO: 83 - protein, 5 aa, Synthetic construct
SYAMG

SEQ ID NO: 84 - protein, 16 aa, Synthetic construct
IIVSDGRTYYTSWAKG

SEQ ID NO: 85 - protein, 9 aa, Synthetic construct
EGYGYYFDI

SEQ ID NO: 86 - protein, 11 aa, Synthetic construct
TADTLSRRYAS

SEQ ID NO: 87 - protein, 7 aa, Synthetic construct
RDTSRPS

SEQ ID NO: 88 - protein, 13 aa, Synthetic construct
ATADGSGSSYQFV

SEQ ID NO: 89 - protein, 117 aa, Synthetic construct
AQSVKESEGGLFKPTDTLTLTCTVSGFSLSSYAISWVRRAPGNGLEWIGAIAGYGDAYYASWAKSRSTITRNTNENTVTLKMTSLTAADTATYFCARDDYNVDTGIWGPGTLVTISS

SEQ ID NO: 90 - protein, 111 aa, Synthetic construct
SFVLTQPASVQVNLGQTVSLTCTADTLSRRYASWYQQKPGQAPVLLIYRDTSRPSGVPDRFSGSSSGNTATLTISGAQAGDEADYYCATGDGSGSSYQYVFGGGTQLTVTG

SEQ ID NO: 91 - protein, 5 aa, Synthetic construct
SYAIS

SEQ ID NO: 92 - protein, 50 aa, Synthetic construct
SEQIDNOPKABATCDRHAMINOACIDSEQUENCEAIAGYGDAYYASWAKS

SEQ ID NO: 93 - protein, 9 aa, Synthetic construct
DDYNVDTGI

SEQ ID NO: 94 - protein, 11 aa, Synthetic construct
TADTLSRRYAS

SEQ ID NO: 95 - protein, 7 aa, Synthetic construct
RDTSRPS

SEQ ID NO: 96 - protein, 13 aa, Synthetic construct
ATGDGSGSSYQYV

SEQ ID NO: 97 - protein, 116 aa, Synthetic construct
AQSLEESGGRLVTPGTPLTLTCTASGFTLSSYHMSWVRQAPGKGLEWIGIISSGGSIYYASWAKGRFTISRTSTTVDLKMTSPTTEDTATYFCARYAAYSIGMDLWGPGTLVTVSS

SEQ ID NO: 98 - protein, 111 aa, Synthetic construct
QFVLTQPASVQVNLGQMVSLTCTADTLSRSYASWYQQKPGQAPVLLIYRDTSRPSGVPDRFSGSTSGNTATLTISGAQAGDEADYYCATDDNSGSSYQYVFGGGTQLTVTG

SEQ ID NO: 99 - protein, 5 aa, Synthetic construct
SYHMS

SEQ ID NO: 100 - protein, 16 aa, Synthetic construct
IISSGGSIYYASWAKG

SEQ ID NO: 101 - protein, 10 aa, Synthetic construct
YAAYSIGMDL

SEQ ID NO: 102 - protein, 11 aa, Synthetic construct
TADTLSRSYAS

SEQ ID NO: 103 - protein, 7 aa, Synthetic construct
RDTSRPS

SEQ ID NO: 104 - protein, 13 aa, Synthetic construct
ATDDNSGSSYQYV

SEQ ID NO: 105 - protein, 114 aa, Synthetic construct
QEQLEESGGRLVTPGTPLTLTCTASGFSLSSYHMSWVRQAPGKGLEWIGLINTGDSAYYASWAKGRFTISRTSTTVDLRITSPTTEDTATYFCARTFGDGFNLWGPGTLVTISS

SEQ ID NO: 106 - protein, 111 aa, Synthetic construct
SFVLTQPASVQVDLGQTVSLTCTADTLSRSYASWYQQKPGQAPVLLIYRDTSRPSGVPDRFSGSSSGNTATLTISGAQAGDEADYYCATEDSGDSSYQYVFGGGTQLTVTG

SEQ ID NO: 107 - protein, 5 aa, Synthetic construct
SYHMS

SEQ ID NO: 108 - protein, 16 aa, Synthetic construct
LINTGDSAYYASWAKG

SEQ ID NO: 109 - protein, 8 aa, Synthetic construct
TFGDGFNL

SEQ ID NO: 110 - protein, 11 aa, Synthetic construct
TADTLSRSYAS

SEQ ID NO: 111 - protein, 7 aa, Synthetic construct
RDTSRPS

SEQ ID NO: 112 - protein, 13 aa, Synthetic construct
ATEDSGDSSYQYV

SEQ ID NO: 113 - protein, 114 aa, Synthetic construct
AQSLEESGGRLVTPGTPLTLTCTASGFSLSSYHMAWVRQAPGKGLEWIGIISSSGSTYYASWAKGRFTISKTSTTVDLKITSPTTEDTATYFCARGFGWGPNLWGPGTLVTVSS

SEQ ID NO: 114 - protein, 111 aa, Synthetic construct
SFVLTQPASVQVNLGQMVSLTCTADTLSRSYASWYQQKPGQAPVLLIYRDTSRPSGVPDRFSGSTSGNTATLTISGAQAGDEADYYCATDDNSGSSYQYVFGGGTQLTVTG

SEQ ID NO: 115 - protein, 5 aa, Synthetic construct
SYHMA

SEQ ID NO: 116 - protein, 16 aa, Synthetic construct
IISSSGSTYYASWAKG

SEQ ID NO: 117 - protein, 8 aa, Synthetic construct
GFGWGPNL

SEQ ID NO: 118 - protein, 11 aa, Synthetic construct
TADTLSRSYAS

SEQ ID NO: 119 - protein, 7 aa, Synthetic construct
RDTSRPS

SEQ ID NO: 120 - protein, 13 aa, Synthetic construct
ATDDNSGSSYQYV

SEQ ID NO: 121 - protein, 115 aa, Synthetic construct
QEQLKESGGRLVTPGTPLTLTCTASGFTISSYHMSWVRQAPGKGLEWIGVISASGGSTYYASWAKGRFTISKTSTTVDLKITSPTTEDTATYFCARESYGGYNLWGPGTLVTVSS

SEQ ID NO: 122 - protein, 111 aa, Synthetic construct; identical to SEQ ID NO: 114

SEQ ID NO: 123 - protein, 5 aa, Synthetic construct
SYHMS

SEQ ID NO: 124 - protein, 17 aa, Synthetic construct
VISASGGSTYYASWAKG

SEQ ID NO: 125 - protein, 8 aa, Synthetic construct
ESYGGYNL

SEQ ID NO: 126 - protein, 11 aa, Synthetic construct
TADTLSRSYAS

SEQ ID NO: 127 - protein, 7 aa, Synthetic construct
RDTSRPS

SEQ ID NO: 128 - protein, 13 aa, Synthetic construct
ATDDNSGSSYQYV

SEQ ID NO: 129 - protein, 115 aa, Synthetic construct
AQSLEESGGRLVTPGTPLTLTCTVSGIDLSSYHMNWVRQAPGKGLEWIGIIGASAGTYYARWAKGRFTISKTSTTVDLKITSPTTEDTATYFCARSGGSMQLNLWGPGTLVTISS

SEQ ID NO: 130 - protein, 111 aa, Synthetic construct
SFVLTQPASVQVNLGQTVSLTCTADTLSRSYASWYQQKPGQAPVLLIYRDTSRPSGVPDRFSGSSSGNTATLTISGAQAGDEADYYCATDDSSGSSYQYVFGGGTQLTVTG

SEQ ID NO: 131 - protein, 5 aa, Synthetic construct
SYHMN

SEQ ID NO: 132 - protein, 16 aa, Synthetic construct
IIGASAGTYYARWAKG

SEQ ID NO: 133 - protein, 9 aa, Synthetic construct
SGGSMQLNL

SEQ ID NO: 134 - protein, 11 aa, Synthetic construct
TADTLSRSYAS

SEQ ID NO: 135 - protein, 7 aa, Synthetic construct
RDTSRPS

SEQ ID NO: 136 - protein, 13 aa, Synthetic construct
ATDDSSGSSYQYV

SEQ ID NO: 137 - protein, 119 aa, Synthetic construct
QEQLEESGGRLVTPGTPLTLTCTASGFSLTNYYMTWVRQAPGKGLEWIGTIYAGTGSTWYASWVKGRFTISKTSTTVDLKITSPTTEDTATYFCVRGYLGWSSGIENIWGPGTLVTVSS

SEQ ID NO: 138 - protein, 111 aa, Synthetic construct; identical to SEQ ID NO: 2

SEQ ID NO: 139 - protein, 5 aa, Synthetic construct
NYYMT

SEQ ID NO: 140 - protein, 17 aa, Synthetic construct
TIYAGTGSTWYASWVKG

SEQ ID NO: 141 - protein, 12 aa, Synthetic construct
GYLGWSSGIENI

SEQ ID NO: 142 - protein, 11 aa, Synthetic construct
TADTLSRSYAS

SEQ ID NO: 143 - protein, 7 aa, Synthetic construct
RDTSRPS

SEQ ID NO: 144 - protein, 13 aa, Synthetic construct
ATSDGSGSTFHFV

SEQ ID NO: 145 - protein, 1445 aa, Homo sapiens
MRRLLEPCWWILFLKITSSVLHYVVCFPALTEGYVGALHENRHGSAVQIRRRKASGDPYWAYSGAYGPEHWVTSSVSCGGRHQSPIDILDQYARVGEEYQELQLDGFDNESSNKTWMKNTGKTVAILLKDDYFVSGAGLPGRFKAEKVEFHWGHSNGSAGSEHSINGRRFPVEMQIFFYNPDDFDSFQTAISENRIIGAMAIFFQVSPRDNSALDPIIHGLKGVVHHEKETFLDPFVLRDLLPASLGSYYRYTGSLTTPPCSEIVEWIVFRRPVPISYHQLEAFYSIFTTEQQDHVKSVEYLRNNFRPQQRLHDRVVSKSAVRDSWNHDMTDFLENPLGTEASKVCSSPPIHMKVQPLNQTALQVSWSQPETIYHPPIMNYMISYSWTKNEDEKEKTFTKDSDKDLKATISHVSPDSLYLFRVQAVCRNDMRSDFSQTMLFQANTTRIFQGTRIVKTGVPTASPASSADMAPISSGSSTWTSSGIPFSFVSMATGMGPSSSGSQATVASVVTSTLLAGLGFGGGGISSFPSTVWPTRLPTAASASKQAARPVLATTEALASPGPDGDSSPTKDGEGTEEGEKDEKSESEDGEREHEEDGEKDSEKKEKSGVTHAAEERNQTEPSPTPSSPNRTAEGGHQTIPGHEQDHTAVPTDQTGGRRDAGPGLDPDMVTSTQVPPTATEEQYAGSDPKRPEMPSKKPMSRGDRFSEDSRFITVNPAEKNTSGMISRPAPGRMEWIIPLIVVSALTFVCLILLIAVLVYWRGCNKIKSKGFPRRFREVPSSGERGEKGSRKCFQTAHFYVEDSSSPRVVPNESIPIIPIPDDMEAIPVKQFVKHIGELYSNNQHGFSEDFEEVQRCTADMNITAEHSNHPENKHKNRYINILAYDHSRVKLRPLPGKDSKHSDYINANYVDGYNKAKAYIATQGPLKSTFEDFWRMIWEQNTGIIVMITNLVEKGRRKCDQYWPTENSEEYGNIIVTLKSTKIHACYTVRRFSIRNTKVKKGQKGNPKGRQNERVVIQYHYTQWPDMGVPEYALPVLTFVRRSSAARMPETGPVLVHCSAGVGRTGTYIVIDSMLQQIKDKSTVNVLGFLKHIRTQRNYLVQTEEQYIFIHDALLEAILGKETEVSSNQLHSYVNSILIPGVGGKTRLEKQFKLVTQCNAKYVECFSAQKECNKEKNRNSSVVPSERARVGLAPLPGMKGTDYINASYIMGYYRSNEFIITQHPLPHTTKDFWRMIWDHNAQIIVMLPDNQSLAEDEFVYWPSREESMNCEAFTVTLISKDRLCLSNEEQIIIHDFILEATQDDYVLEVRHFQCPKWPNPDAPISSTFELINVIKEEALTRDGPTIVHDEYGAVSAGMLCALTTLSQQLENENAVDVFQVAKMINLMRPGVFTDIEQYQFIYKAMLSLVSTKENGNGPMTVDKNGAVLIADESDPAESMESLV

SEQ ID NO: 146 - protein, 538 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 147 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 148 - protein, 1442 aa, Mus musculus
MRRLLEPCWWILFLKITSSVLHYVVCFPALTEGYVGTLQESRQDSSVQIRRRKASGDPYWAYSGAYGPEHWVTSSVSCGGSHQSPIDILDHHARVGDEYQELQLDGFDNESSNKTWMKNTGKTVAILLKDDYFVSGAGLPGRFKAEKVEFHWGHSNGSAGSEHSVNGRRFPVEMQIFFYNPDDFDSFQTAISENRIIGAMAIFFQVSPRDNSALDPIIHGLKGVVHHEKETFLDPFILRDLLPASLGSYYRYTGSLTTPPCSEIVEWIVFRRPVPISYHQLEAFYSIFTTEQQDHVKSVEYLRNNFRPQQALNDRVVSKSAVRDAWNHDLADFLDNPLGTEASKVCSSPPIHMKVQPLNQTALQVSWSQPETIYHPPIMNYMISYSWTKNEDEKEKTFTKDSDKDLKATISHVSPDSLYLFRVQAVCRNDMRSDFSQTMLFQANTTRIFQGTRIVKTGVPTASPASSADMAPISSGSSTWTSSGIPFSFVSMATGMGPSSSGSQATVASVVTSTLLAGLGFGGGGISSFPSTVWPTRLPTASAASKQAGRTVLATTEALASPGPDVHSAPSKDSEGTEEGEKEEKSESEDGEREHEEEEKDSEKKEKSEATHTAAESDRTAPAPTPSSPHRTAAEGGHQTIPGRRQDHSAPATDQPGHVAPDLDPLVDTATQVPPTATEEHYSGSDPRRPEMPSKKPMSRGDRFSEDSKFITVNPAEKNTSGMLSRPSPGRMEWIIPLIVVSALTFVCLVLLIAVLVYWRGCNKIKSKGFPRRSREVPSSGERGEKGSRKCFQTAHFYVEDSSSPRVVPNESVPIIPIPDDMEAIPVKQFGKHIGELYSNSQHGFSEDFEEVQRCTADMNITAEHSNHPDNKHKNRYINILAYDHSRVKLRPLPGKDSKHSDYINANYVDGYNKAKAYIATQGPLKSTFEDFWRMIWEQNTGIIIMITNLVEKGRRKCDQYWPTENTEEYGNIIVTLKSTKVHACYTVRRLSVRNTKVKKGQKGNPKGRQNERTVIQYHYTQWPDMGVPEYALPVLTFVRRSSAARMPDMGPVLVHCSAGVGRTGTYIVIDSMLQQIKDKSTVNVLGFLKHIRTQRNYLVQTEEQYIFIHDALLEAILGKETAVSSSQLHSYVNSILIPGVGGKTRLEKQFKLITQCNAKYVECFSAQKECNKEKNRNSSVVPAERARVGLAPLPGMKGTDYINASYIMGYYRSNEFIITQHPLPHTTKDFWRMIWDHNAQIIVMLPDNQSLAEDEFVYWPSREESMNCEAFTVTLISKDRLCLSNEEQIIIHDFILEATQDDYVLEVRHFQCPKWPNPDAPISSTFELINVIKEEALTRDGPTIVHDEYGAVSAGMLCALTTLSQQLENENAVDVFQVAKMINLMRPGVFTDIEQYQFVYKAMLSLISTKENGNGPMTGDKNGAVLTAEESDPAESMESLV